(19) United States
(12) Patent Application Publication
(10) Pub. No.: US 2022/0339193 A1
(43) Pub. Date: Oct. 27, 2022

(54) Modified Cell Expressing Therapeutic Agent and Uses thereof

(71) Applicant: Innovative Cellular Therapeutics Holdings, Ltd., Rockville, MD (US)

(72) Inventors: Lei Xiao, Rockville, MD (US); Chengfei Pu, Shanghai (CN); Zhiyuan Cao, Shanghai (CN)

(73) Assignee: Innovative Cellular Therapeutics Holdings, Ltd., Rockville, MD (US)

(21) Appl. No.: 17/749,824

(22) Filed: May 20, 2022

Related U.S. Application Data
(63) Continuation of application No. 17/295,364, filed on May 19, 2021.
Application No. 17/295,364 is a continuation of application No. 16/445,965, filed on Jun. 19, 2019, now Pat. No. 10,918,667.
(60) Provisional application No. 62/902,766, filed on Sep. 19, 2019.
(60) Provisional application No. 62/889,926, filed on Aug. 21, 2019.
(60) Provisional application No. 62/848,961, filed on May 16, 2019.
(60) Provisional application No. 62/846,563, filed on May 10, 2019.
(60) Provisional application No. 62/828,770, filed on Apr. 3, 2019.
(60) Provisional application No. 62/795,810, filed on Jan. 23, 2019.
(60) Provisional application No. 62/774,595, filed on Dec. 3, 2018.
(60) Provisional application No. 62/769,987, filed on Nov. 20, 2018.

(51) Int. Cl. A61K 35/17; C07K 14/54; C07K 14/57; C07K 14/725; C07K 14/705; C07K 16/28

(52) U.S. Cl. CPC A61K 35/17; C07K 14/5412; C07K 14/57; C07K 14/7051; C07K 14/70521; C07K 14/70578; C07K 16/2803; A61K 2039/505

(57) ABSTRACT

Compositions and methods for enhancing T cell response which increases the efficacy of CAR T cell therapy for treating cancer are described. Embodiments include a modified cell comprising an isolated nucleic acid comprising a first nucleic acid and a second nucleic acid, the first nucleic acid encoding a chimeric antigen receptor (CAR), the second nucleic acid encoding a therapeutic agent comprising at least one of IFN-γ, IL-2, IL-6, IL-7, IL-15, IL-17, and IL-23. The modified cell expresses and secretes the therapeutic agent.

### CROSS REFERENCE TO RELATED APPLICATIONS

This application is a continuation of co-pending U.S. patent application Ser. No. 17/295,364 filed May 19, 2021, which is a U.S. National Phase Application of International Application No. PCT/US2019/062417, filed Nov. 20, 2019, which is a continuation of U.S. patent application Ser. No. 16/445,965, filed Jun. 19, 2019; and which claims the benefit of U.S. Provisional Application No. 62/848,961, filed May 16, 2019; U.S. Provisional Application No. 62/846,563, filed May 10, 2019; U.S. Provisional Application No. 62/828,770, filed Apr. 3, 2019; U.S. Provisional Application No. 62/795,810, filed Jan. 23, 2019; U.S. Provisional Application No. 62/774,595, filed Dec. 3, 2018; and U.S. Provisional Application No. 62/769,987, filed Nov. 20, 2018, which are incorporated by reference herein in their entirety. U.S. patent application Ser. No. 17/295,364 also claims the benefit of U.S. Provisional Application No. 62/902,766, filed Sep. 19, 2019; and U.S. Provisional Application No. 62/889,926, filed Aug. 21, 2019; all of which are incorporated by reference herein in their entirety.

### SEQUENCE LISTING INFORMATION

A computer readable textfile, entitled “SDS1.0072US3_ST25.txt,” created on or about Jun. 29, 2022, with a file size of about 1,378,231 bytes, contains the sequence listing for this application and is hereby incorporated by reference in its entirety.

### TECHNICAL FIELD

The present disclosure relates to compositions and methods related to chimeric antigen receptor cells secreting therapeutic agents and uses thereof in the treatment of diseases, including cancer.

### BACKGROUND

Cancer involves abnormal cell growth with the potential to invade or spread to other parts of the body. In humans, there are more than one hundred types of cancer. One example is breast cancer occurring in the epithelial tissue of the breast. Since breast cancer cells lose the characteristics of normal cells, the connection between breast cancer cells is lost. Once cancer cells are exfoliated, they spread over the entire body via the blood and/or lymph systems and therefore become life-threatening. Currently, breast cancer has become one of the common threats to women's physical and mental health. Although immunotherapy, for example, CAR T cell therapy, has been proven to be effective for treating cancer, there is still a need to improve such immunotherapy so that it is more effective for certain cancers such as those involving solid tumors.

### SUMMARY

The present disclosure describes compositions and methods for enhancing T cell response. The present disclosure also describes cells comprising an isolated nucleic acid comprising a nucleic acid and an additional nucleic acid, the nucleic acid encoding a chimeric antigen receptor (CAR), the additional nucleic acid encoding a therapeutic agent comprising at least one of IFN-γ, IL-2, IL-6, IL-7, IL-15, IL-17, IL-12, and IL-23. The cells may conditionally express and secrete the therapeutic agent.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram of exemplary fusion proteins including GS linker as set forth in SEQ ID NO: 536.

FIG. 2 shows a schematic diagram of exemplary fusion proteins including GS linker as set forth in SEQ ID NO: 536.

FIG. 3 shows a schematic diagram of exemplary fusion proteins.

FIG. 4 shows a schematic diagram of exemplary fusion proteins.

FIG. 5 shows a schematic diagram of an exemplary CAR molecule and a fusion protein.

FIG. 6 shows a schematic diagram of an exemplary CAR molecule and a protein expressed by a modified cell.

FIG. 7 shows a schematic diagram of an exemplary CAR molecule and one or more proteins expressed by a modified cell.

FIG. 8 shows a schematic diagram of an exemplary CAR molecule and one or more proteins expressed by a modified cell.

FIG. 9 shows a schematic diagram of an exemplary CAR molecule and one or more proteins (Agent 1 and Agent 2) expressed by a modified cell.

FIG. 10 shows a schematic diagram of an exemplary CAR molecule and one or more proteins expressed by a modified cell.

FIGS. 11, 12, and 13 show cytokines released in response to the infusion of CD19CAR T cells for treating B-ALL (B-Cell Acute Lymphoblastic Leukemia).

FIGS. 14 and 15 show results of flow cytometry assay indicating macrophage phenotype changes.

FIG. 16 shows results of cytometry assay indicating macrophage phenotype changes after the macrophages were co-cultured with various CAR T cells expressing IFN-γ and/or IL-6.

FIG. 17 show schematic diagrams of various constructs of CAR and therapeutic agents expressed by modified cells.

FIG. 18 shows results of flow cytometry assay of T cells expressing various proteins shown in FIG. 17.

FIG. 19 shows the release of IL-6 by modified cells in response to CD3/CD28 Dynabeads activation.

FIG. 20 shows the release of IL-6 by modified cells in response to co-culturing with Nalm6 cells.

FIG. 21 shows the release of IFNγ by modified cells in response to CD3/CD28 Dynabeads activation.

FIG. 22 shows the release of IFNγ (IFNg) by modified cells in response to co-culturing with Nalm6 cells.

FIG. 23 shows the killing assay results of various CAR T cells.

FIGS. 24 and 25 show the expression of certain proteins on modified cells and the release of IFNγ by modified cells in response to co-culturing with Nalm6 cells.

FIGS. 26 and 27 show the expression of certain proteins on modified cells and the release of IL-12 and IFNγ by modified cells in response to CD3/CD28 Dynabeads activation.

FIGS. 28 and 29 show the expression of certain proteins on modified cell and the release of IL-6 and IFNγ by modified cells in response to CD3/CD28 Dynabeads activation.

FIG. 30 shows the results of the anaerobic assay on various CAR T cells.

FIG. 31 shows cytokines released in response to hypoxia in the TSHR-CART system.

FIGS. 32 and 33 show cytokines released in response to the induction of IL-12 expression in CAR T cells.

FIG. 34 shows cytokines released in response to hypoxia in the GUCY2C-CART system.

FIG. 35 shows IFNγ release in response to the induction of IL-12 expression in CAR T cells.

FIG. 36 shows the release of IL-6, TNFα, IFNγ, and GZMB induced by T cell activation in the ACPP-CART system.

FIGS. 37 and 38 show cytokines released by modified cells in response to hypoxia in the ACPP-CART system.

FIG. 39 shows the results of cytokine release assay indicating that IL-6 and IFNγ are released by various types of CAR T cells in different conditions after the CAR T cells were cultured with or without antigen for 24 hours.

FIGS. 40, 41, 42, and 43 show levels of cytokines released and other parameters in response to CAR T cell infusion in Patient 005.

FIGS. 44 and 45 show various parameters in response to CAR T cell infusion in Patient 004.

FIGS. 46 and 47 show cytokines released in response to the infusion of CAR T cells on Patient 006.

FIG. 48 shows tumor changes before and after the CAR T cell infusion of Patient 005 by PET/CT images.

FIG. 49 shows tumor changes before and after the CAR T cell infusion of Patient 006 by CT images.

FIGS. 50 and 51 show comparisons of cytokines released among Patients 004, 005, and 006.

FIG. 52 shows the expansion of T cells in each of groups.

FIG. 53 shows tumor changes before and after the CAR T cell infusion of Patient 007 by PET/CT images.

FIG. 54 show cytokines released in response to the infusion of CAR T cells on Patient 007.

FIG. 55 shows a schematic diagram of an exemplary modified cell.

FIG. 56 shows a schematic diagram an exemplary population of modified cells.

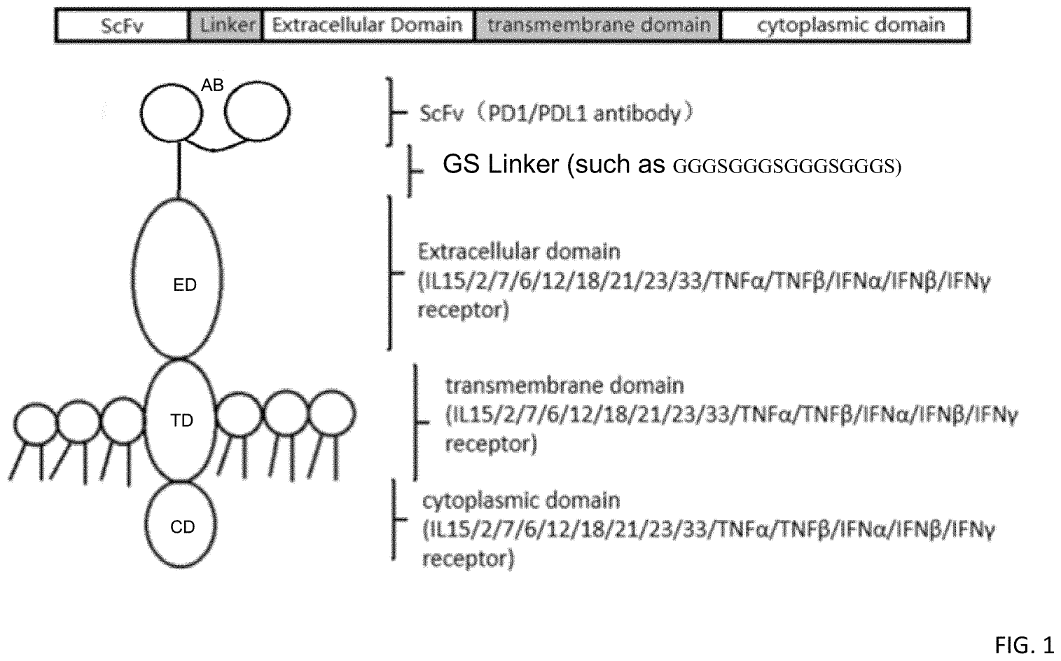

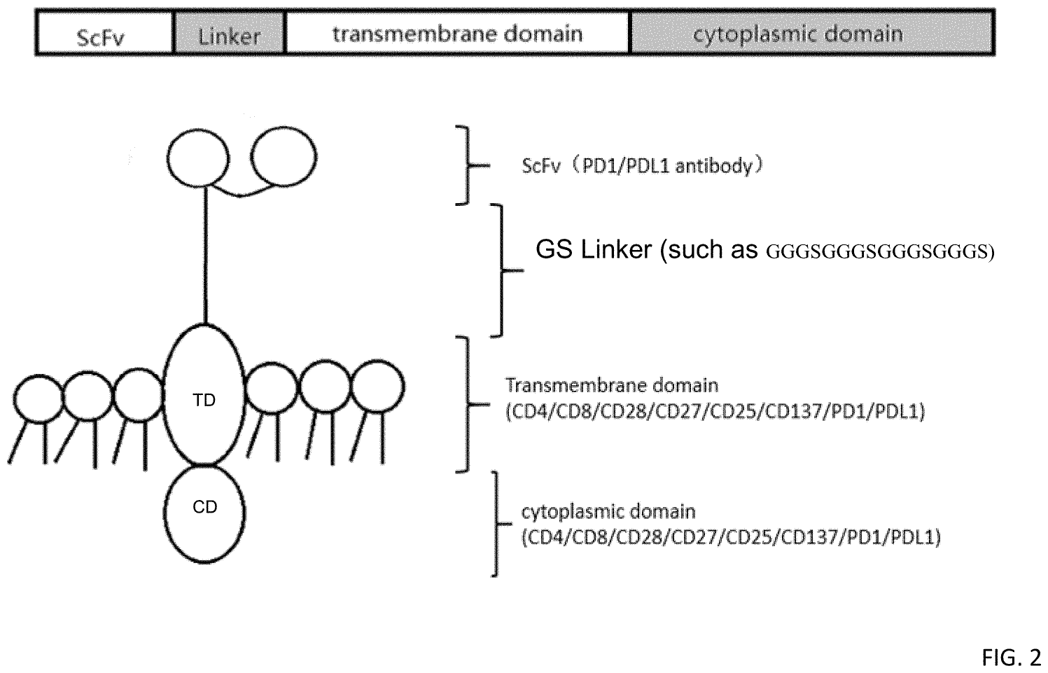

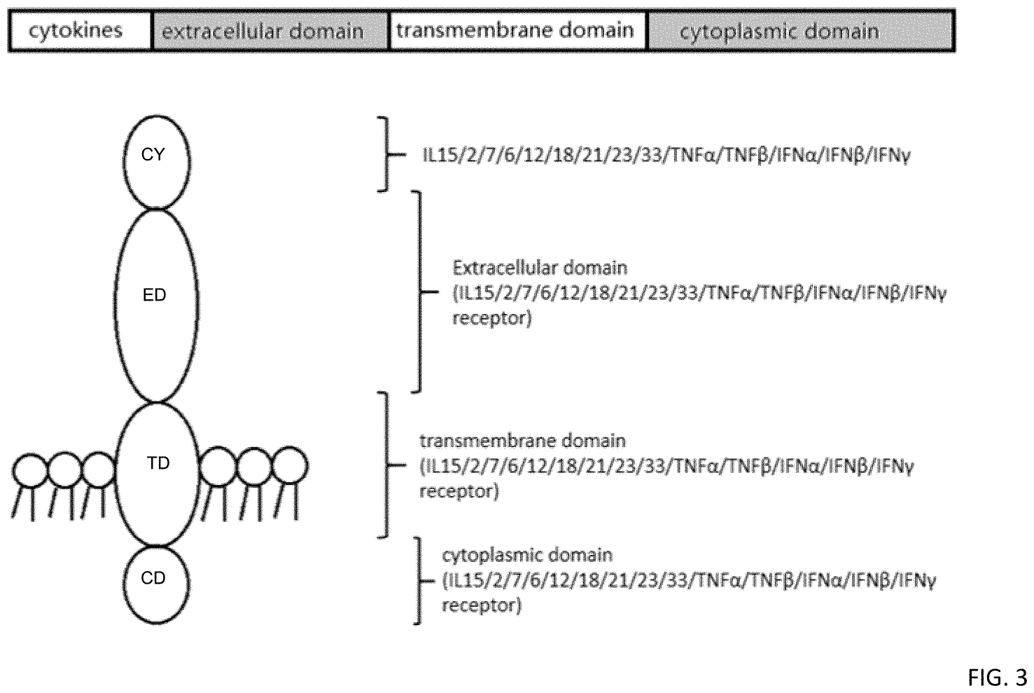

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab)₂ and fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragments” refers to a portion of a full-length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumors in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject that is subsequently re-introduced into the same subject.

The term “allogeneic” is used to describe a graft derived from a different subject of the same species. As an example, a donor subject may be a related or unrelated or recipient subject, but the donor subject has immune system markers that are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base-pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand,” refers to a molecule on an antigen-presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules. The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated, then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression or overexpression” refers to the transcription and/or translation of a particular nucleotide sequence into a precursor or mature protein, for example, driven by its promoter. “Overexpression” refers to the production of a gene product in transgenic organisms or cells that exceeds levels of production in normal or non-transformed organisms or cells. As defined herein, the term “expression” refers to expression or overexpression.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous, then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that are normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables the integration of the genetic information into the host chromosome, resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response, thereby mediating a beneficial therapeutic response in a subject, preferably, a human.

Nucleic acid is “operably linked” when it is placed into a functional relationship with another polynucleotide. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

Cancers that may be treated include tumors that are not vascularized, or not yet substantially vascularized, as well as vascularized tumors. The cancers may include non-solid tumors (such as hematological tumors, for example, leukemia, lymphoma, and multiple myeloma) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases).

A solid tumor antigen is an antigen expressed on a solid tumor. In embodiments, the solid tumor antigen is also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal cancer |
| MUC12 | colorectal cancer |
| GUCY2C | colorectal cancer |
| GPR35 | colorectal cancer |
| CR1L | Gastric Cancer |
| MUC 17 | Gastric Cancer |
| TMPRSS11B | esophageal cancer |
| MUC21 | esophageal cancer |
| TMPRSS11E | esophageal cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial |
| | cancer |
| UPK2 | Urothelial cancer (including bladder |
| | cancer) |
| ADAM12 | Breast cancer, pancreatic cancer |
| | and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, Pancreatic |
| | cancer, Breast Cancer |
| B7-H3 | Ewing sarcoma (bone tumor), |
| | rhabdomyosarcoma, nephroblastoma, |
| | neuroblastoma and medulloblastoma |
| | (brain tumor) |

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein and refer to any human, or animal, amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for the prevention of a disease, condition, or disorder. In embodiments, the disease, condition, or disorder is cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double-stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control (regulatory) sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.5 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures.

The term “stimulatory molecule” refers to a molecule on a T cell that specifically binds a cognate stimulatory ligand present on an antigen-presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen-presenting cell (e.g., an APC, a dendritic cell, a B-cell, and the like) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, and other factors, of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate the transfer of nucleic acid into cells, such as, for example, polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

A “chimeric antigen receptor” (CAR) molecule is a recombinant polypeptide including at least an extracellular domain, a transmembrane domain, and a cytoplasmic domain or intracellular domain. In embodiments, the domains of the CAR are on the same polypeptide chain, for example, a chimeric fusion protein. In embodiments, the domains are on different polypeptide chains, for example, the domains are not contiguous.

The extracellular domain of a CAR molecule includes an antigen binding domain. In embodiments, the antigen binding domain binds an antigen, for example, a cell surface molecule or marker, on the surface of a B cell. In embodiments, the cell surface molecule of a B cell includes CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the B cell is CD19, CD20, CD22, or BCMA. In particular embodiments, the cell surface molecule of the B cell is CD19.

In embodiments, the antigen binding domain binds an antigen, on the surface of a tumor, for example, a tumor antigen or tumor marker. Tumor antigens are proteins that are produced by tumor cells that elicit an immune response, particularly T cell-mediated immune responses. Tumor antigens are well known in the art and include, for example, tumor-associated MUC1 (tMUC1), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the tumor antigen is CD19, and the CAR thereof can be referred to as CD19CAR and the T cell comprising CD19CAR can be referred to a CART19 cell or CD19CAR T cell.

In embodiments, the extracellular antigen binding domain of a CAR includes at least one scFv or at least a single domain antibody. As an example, there can be two scFvs on a CAR. The scFv includes a light chain variable (VL) region and a heavy chain variable (VH) region of a target antigen-specific monoclonal antibody joined by a flexible linker. Single chain variable region fragments can be made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 124), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the CAR molecules described herein comprises one or more CDRs for binding an antigen of interest, for example, one or more CDRs for binding CD19 or tMUC1.

The cytoplasmic domain of the CAR molecules described herein includes one or more co-stimulatory domains and one or more signaling domains. The co-stimulatory and signaling domains function to transmit the signal and activate molecules, such as T cells, in response to antigen binding. The one or more co-stimulatory domains are derived from stimulatory molecules and/or co-stimulatory molecules, and the signaling domain is derived from a primary signaling domain, such as the CD3 zeta domain. In embodiments, the signaling domain further includes one or more functional signaling domains derived from a co-stimulatory molecule. In embodiments, the co-stimulatory molecules are cell surface molecules (other than antigens receptors or their ligands) that are required for activating a cellular response to an antigen.

In embodiments, the co-stimulatory domain includes the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds CD83, or any combination thereof. In embodiments, the signaling domain includes a CD3 zeta domain derived from a T cell receptor.

In embodiments, the cytoplasmic domain of the CAR only includes one or more stimulatory domains and no signaling domain.

The CAR molecules also include a transmembrane domain. The incorporation of a transmembrane domain in the CAR molecules stabilizes the molecule. In embodiments, the transmembrane domain of the CAR molecules is the transmembrane domain of a CD28 or 4-1BB molecule.

Between the extracellular domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain on the polypeptide chain. A spacer domain may include up to 300 amino acids, preferably 10 to 100 amino acids, and most preferably 25 to 50 amino acids.

Although immunotherapy using CAR T has brought hope to many patients to cure their cancer, CAR T therapy can cause cytokine storm (or cytokine release syndrome (CRS)). Cytokine storm refers to the activation and rapid proliferation of T lymphocytes in vivo after completion of CAR T infusion in humans, causing excessive release of TNF-α, IFN-γ, IL-2, IL-4, IL-6, IL.-8, IL-10 and other cytokines. These cytokines mediate a variety of immune responses, causing high-grade fever, hypotension, myalgia, coagulopathy, dyspnea, end-organ disorder and other clinical manifestations, which may cause serious permanent damage or failure of the human tissues and organs. However, activation and rapid proliferation of lymphocytes are closely related to the efficacy of immunotherapy, and cytokines appear to be involved in the activation and proliferation of lymphocytes. Embodiments described herein relate to the discovery that lymphocytes may be engineered to conditionally express and/or secrete one or more cytokines to have enhanced but manageable immune response in a subject. In embodiments, the expression and/or secretion of the one or more cytokines may be regulated by one or more conditions, such as the presence of an antigen that the lymphocytes recognize, the level of oxygen (e.g., hypoxia), the level of pH values, the presence of a drug (e.g., doxycycline for a rtTA-TRE system), the presence of a transcription factor in immune response, and other immune associated conditions.

### CAR Molecule(s) and Therapeutic Agent(s)

The present disclosure describes isolated nucleic acids including a (first) nucleic acid encoding a CAR and an additional (second) nucleic acid encoding one or more therapeutic agents. In embodiments, the first nucleic acid and the second nucleic acid are on separate isolated nucleic acids. In embodiments, the first and second nucleic acid are on the same isolated nucleic acid. In embodiments, the one or more therapeutic agents comprise at least one cytokine, or a derivative thereof. Different types of cytokines have been discovered, including chemokines, interferons (IFNs), interleukins (ILs), lymphokines, and tumor necrosis factors (TNFs).

In embodiments, the one or more therapeutic agents comprise at least one of IFN-γ, IL-2, IL-6, IL-7, IL-15, IL-17, IL-23, or derivatives thereof. In embodiments, the therapeutic agent is Eomes, TRAF6, IL12, IL2, IL18, IL23, AQP9, Runx3, AMPK, BCL-2, or a combination thereof.

The present disclosure also describes isolated nucleic acids including a first nucleic acid encoding a first CAR and a second nucleic acid encoding the one or more therapeutic agents. Moreover, the present disclosure describes isolated nucleic acids including a third nucleic acid encoding a second (or additional) CAR. In embodiments, a separate isolated nucleic acid includes a nucleic acid encoding the second CAR. In embodiments, the first nucleic acid and the second nucleic acid are on separate isolated nucleic acids.

In embodiments, the polynucleotide may integrate into the genome of the modified cell and descendants of the modified cell will also express the polynucleotide, resulting in a stably transfected modified cell. In embodiments, the modified cell may express the polynucleotide encoding the CAR but the polynucleotide does not integrate into the genome of the modified cell such that the modified cell expresses the transiently transfected polynucleotide for a finite period of time, for example a few days, after which the polynucleotide is lost through cell division or other cellular processes. As an example, the polynucleotide is present in the modified cell in a recombinant DNA construct, in an mRNA, or in a viral vector, and/or the polynucleotide is an mRNA, which is not integrated into the genome of the modified cell.

Embodiments relate to a method or use of the polynucleotides described herein. The method or use includes: providing a viral particle (e.g., AAV, lentivirus or their variants) comprising a vector genome, the vector genome comprising the polynucleotide, wherein the polynucleotide is operably linked to an expression control element conferring transcription of the polynucleotide; and administering an amount of the viral particle to the subject such that the polynucleotide is expressed in the subject. In embodiments, the AAV preparation includes AAV vector particles, empty capsids, and host cell impurities, thereby providing an AAV product substantially free of AAV empty capsids. More information of the administration and preparation of the viral particle may be found at the U.S. Pat. No. 9,840,719 and Milani et al., Sci. Transl. Med. 11, eaav7325 (2019) 22 May 2019, which are incorporated herein by reference.

In embodiments, a bioreactor can be inoculated at a cell density of approximately 0.5×10⁶ cells/mL with viability greater than 95%. When the cell density reaches approximately 1.0×10⁶ cells/mL, the cells may be transfected with the polyethyleneimine (PEI)/DNA complexes (polyplexes) with a PEI to DNA ratio of 2:1. At the time of harvest, AAV from the cell culture in the bioreactor may be released using the Triton X-100 method. All solutions may be added directly to the bioreactor, and the lysate centrifuged at 4000×g for 20 min. The supernatant can be stored at −80° C. for further processing. AAV may be further purified. For example, AAV samples (12.3 mL) may be purified by overlaying them on top of a series of step gradients using 15, 25, 40 and 54% iodixanol concentrations containing 1, 5, 7 and 5 mL, respectively. The 15% iodixanol concentration also contains 1 M NaCl to avoid aggregation of AAV with other cellular proteins and negatively charged nuclear components. After the completion of centrifugation, 5 mL may be withdrawn from 2 mm below the 40/54 interface marked before starting the ultracentrifugation at 385,000×g for 1 h 45 min in Sorvals T-865 rotor in Sorval Ultracentrifuge. The viral vectors can then be quantified. For example, vectors AAV infectivity can be determined by the gene transfer assay (GTA) using GFP as a reporter gene in all cases. AAV infectivity assay, in which samples are diluted before addition to the cells, have the GFP positive cells in the range of 2-20% to ensure that only a single virus has entered the cell for GFP expression. The GFP-positive cells may be quantified by FACS using HEK293 cells in suspension. The AAV may be then administrated to a subject. For example, AAV may be diluted in 0.9% sterile NaCl saline solution (supplemented with 0.25% human serum albumin [HSA]) for infusion in patients and the final volume of infusion can be calculated based on the patient's weight as 3 mL/kg.

In embodiments, that first CAR includes an antigen binding domain that binds a solid tumor, and the second (or additional) CAR includes an antigen binding domain that binds a white blood cell (WBC).

The present disclosure also describes vectors including the isolated nucleic acids described herein. In embodiments, a single vector contains the isolated nucleic acid encoding the first CAR, the therapeutic agent, and the second CAR or TCR. In embodiments, a first vector contains the first nucleic acid encoding a first CAR and a nucleic acid encoding one or more therapeutic agents, and a second vector contains the nucleic acid encoding the second CAR or TCR. In embodiments, the vector comprises an isolated nucleic acid encoding a bispecific CAR including at least two antigen binding domains and one or more therapeutic agents.

In embodiments, the present disclosure describes an isolated polynucleotide comprising a polynucleotide and an additional polynucleotide, the polynucleotide encoding a chimeric antigen receptor (CAR), the additional polynucleotide encoding a therapeutic agent that is or comprises at least one of IL-2, IL-6, IL-7, IL-15, IL-17, and IL-23. In embodiments, the therapeutic agent is or comprises Eomes, TRAF6, IL12, IL2, IL18, IL23, AQP9, Runx3, AMPK, or BCL-2.

In embodiments, the present disclosure describes a pharmaceutical composition for treating a subject having a tumor using modified T cells, wherein the pharmaceutical composition comprises modified T cells comprising a first polynucleotide encoding a chimeric antigen receptor (CAR) and a second polynucleotide encoding a therapeutic agent comprising IL-6, IFN-γ, or a combination thereof. In embodiments, the tumor is a solid tumor. In embodiments, the tumor is a liquid tumor (e.g., NHL). Embodiments relate to a method of causing or inducing T cell response, enhancing T cell therapy, enhancing in vivo T cell expansion, and/or reducing M2 macrophages in a subject in need thereof, the method comprising administering an effective amount of the pharmaceutical composition to the subject. The method described herein is effective in treating a subject diagnosed with cancer. In embodiments, the subject is diagnosed with a solid tumor.

Embodiments relate to certain cytokines, for example, IL-6, IFNγ and IL-12, are selected to be expressed or overexpressed in T cells, which are used to treat tumors (e.g., solid and/or liquid tumors). These cytokines at least do not directly or indirectly weaken the killing function, the capability of inhibiting tumor cells, and/or have severe side effects on T cell therapy. Alternatively, some of these cytokines may directly or indirectly affect the killing function, the capability of inhibiting tumor cells, and/or have severe side effects on T cell therapy. However, these effects are manageable such as not to expose patients to substantial risk in light of the benefit of T cell therapy. For example, these selected cytokines are capable of enhancing T cell response. Interestingly, IL-6 was considered as a cytokine that reduces or at least has a negative impact on T cell therapy since it is the major contributor to cytokine release syndrome (CRS). However, the Examples provided herein show that the increase of IL-6 is consistent with the efficacy in treating Relapsed/Refractory (R/R) Acute Lymphoid Leukemia (ALL) using CAR T cell therapy. Surprisingly, the Examples provided herein show the infusion of CAR T cells expressing and secreting IL-6 does not cause severe CRS for treating solid tumors. Not all cytokine can be expressed and secreted by T cells without sacrificing T cells' functions to kill tumor cells and/or inhibit tumor growth. The tumor-promoting effects of certain cytokines have been reported. For example, IL-10 production by TAMs can blunt anti-tumor responses by inhibiting the functions of APCs and subsequently block T cell effector functions such as cytotoxicity (Mannino, M. H., Zhu, Z., Xiao, H., Bai, Q., Wakefield, M. R., and Fang, Y. (2015). Studies in mouse tumor models have shown that IL-10 can suppress tumor-infiltrating DC maturation and their production of IL-12 to stimulate Th1 cells, unless IL-10 signaling is simultaneously blocked (Vicari, A. P., Chiodoni, C., Vaure, C., Aüt-Yahia, S., Dercamp, C., Matsos, F., Reynard, O., Taverne, C., Merle, P., Colombo, M. P., et al. (2002). Reversal of tumor-induced dendritic cell paralysis by CpG immunostimulatory oligonucleotide and anti-interleukin 10 receptor antibody. J. Exp. Med. 196, 541-549). As another example, studies have shown that TGF-β can be a potent inhibitor of T cell proliferation (Kehrl J H, Wakefield L M, Roberts A B, Jakowlew S, Alvarez-Mon M, et al. 1986. Production of transforming growth factor β (TGF-β) by human T lymphocytes and its potential role in the regulation of T cell growth. J. Exp. Med. 163:1037-50). Several mechanisms drive TGF-β-mediated inhibition of T cell proliferation, including suppression of IL-2 production, downregulation of c-myc, and upregulation of cyclin-dependent kinase inhibitors (Li M O, Wan Y Y, Sanjabi S, Robertson A K, Flavell R A. 2006. Transforming growth factor-β regulation of immune responses. Annu. Rev. Immunol. 24:99-146). In some contexts, TGF-β also plays an important role in promoting cell death to limit T cell expansion after activation (Mark A. Travis and Dean Sheppard, TGF-β Activation and Function in Immunity, Annu. Rev. Immunol. 2014. 32:51-82). Chemokines are a large family of cytokines that direct normal leukocyte migration. They also have been implicated in leukocyte development and in the pathogenesis of many diseases. Also, some chemokines' concentration gradients play an important role in intranodal T-cell migration. Expression or overexpression of these chemokines would disrupt T cell migration, thus weakening CAR T cell therapy for solid tumors. Without proper migration, T cells may not be able to reach tumor cells. For example, it has been reported that overexpression of the chemokine CCL21 disrupts T cell migration (Christopherson K W and Campbell J J, Hromas R A, Transgenic overexpression of the CC chemokine CCL21 disrupts T-cell migration, Blood. 2001 Dec. 15; 98(13):3562-8). In embodiments, cytokines over-expressed or expressed in the modified cells does not include at least one of IL-10, TGF-β. and CCL21. Certain cytokines can be overexpressed or expressed in T cells to enhance CAR T therapy treating tumors. However, some cytokines (e.g., IL-6) cannot be overexpressed or expressed in T cells to treat blood tumors. It is well-known that IL-6 is the major factor that contributes to severe CRS in CAR T treatment of blood tumors such as ALL and NHL. In contrast, IL-6 can be overexpressed or expressed in T cells for the treatment of solid tumors because the studies showed that few severe CRS in CAR T cell treatment of solid tumors. In embodiments, expression and secretion of IL-6 by T cells may be associated with a condition of T cells to avoid CRS and other syndromes associated with IL-6. For example, IL-6 may be expressed and secreted by T cells when the T cells are activated. In embodiments, expression and secretion of IL-6 by CAR T cells may be regulated by a transcription modulator such as NFAT such that the CAR T cells may neither express nor secrete IL-6 unless they recognize and bind their antigen. In embodiments, the modified cells may express and/or secrete an agent that interferes with the activities of cytokines that reduce anti-tumor activities (e.g., IL-10, TGF-β. and CCL21) using methods described in the present disclosure. In embodiments, the modified cells may express and/or secrete a scFv targeting IL-10 and/or IL-10 receptors or a soluble receptor of IL-10, for example, in response to activation of the modified cells and/or a level of oxygen (e.g., hypoxia). In embodiments, a population of modified cells may include modified cells expressing various combinations of cytokines and the agent. For example, the population of modified cells may include modified cells including a polynucleotide encoding IL-6 and IFNγ, modified cells including a polynucleotide encoding IL-12 and the agent, and/or modified cells including a polynucleotide encoding the agent. In embodiments, the modified cells may express one or more antigen binding molecules (e.g., CAR and TCR). In embodiments, the agent can be a polynucleotide encoding a protein (e.g., dominant negative form of PD-1 and fusion proteins described herein) to overexpress and/or secrete the protein in the modified cell. In embodiments, the polynucleotide can encode a genome editing tool (e.g., ZFN, TALEN, Cas9) such as to reduce a function and/or expression of a target gene in the modified cell. In embodiments, the modified cell may express a dominant negative form of a checkpoint inhibitor (e.g., PD-1). For example, it has been reported that IL-6 may increase PD-1 expression by T cells, and expression of the dominant negative form of PD-1 by the modified cells that also express IL-6 may increase the modified cells' anti-tumor activities.

It has been reported that there are many kinds of cells in solid tumor tissues, including tumor cells and some immune cells. Among them, macrophages, such as M2 macrophages are the main cells of immune cells in the solid tumor tissues. The solid tumor M2 type macrophages are inside tumor tissues secreting cytokines continuously to nourish the tumor cells. Embodiments relate to compositions and methods of reducing the number of M2 macrophages to enhance immunotherapy (e.g., CAR, TCR, and/or TIL) on a subject having cancer. In embodiments a cell (e.g., T cell or NK cell) may be modified to express one or more molecules at a level that is higher than the level of the one or more expressed by a cell that has not been modified to expression the one or more molecules such that delivery of the modified cell can reduce the number of M2 macrophages in tumor microenvironment. For example, the one or more molecules comprise IFN-γ or derivatives thereof, and the expression of IFN-γ is regulated by NFAT.

In embodiments, the modified T cells express and secrete the therapeutic agent. In embodiments, the therapeutic agent comprises IL-6 and IFN-γ. In embodiments, the modified cell comprises a nucleic acid encoding NFAT, IFN-γ, IRES/2a, and IL-6 in such order. In embodiments, the modified T cell comprises polynucleotides encoding SEQ ID NOs: 287 and 328. In embodiments, the modified T cell comprises the polynucleotides comprising SEQ ID NOs: 286 and 469, and a polynucleotide encoding SEQ ID NO: 328.

In embodiments, IFN-γ may comprise wild type IFN-γ, and derivatives of IFN-γ of which certain amino acids of IFN-γ polypeptide are removed and certain amino acids other than the removed amino acids are replaced with other amino acids. The derivatives can show higher IFN-γ activity and/or stability as compared to the wild type. Examples of some of the derivatives are included in U.S. Pat. No. 4,898,931 and US Patent Publication Nos: US2003092130 and US2006251619, which are incorporated herein by reference in their entirety.

In embodiments, the one or more therapeutic agents are IFN-α, IFN-v, IFN-ω, IFN-ε, IFN-κ, IFN-τ, IFN-δ, IFN-ζ, IFN-μ, IRF8, batf3 E2-2, IRF4, and/or Notch2 KLF4. IFN-α proteins are produced mainly by plasmacytoid dendritic cells (pDCs). They are mainly involved in innate immunity against viral infection. The genes responsible for their synthesis come in 13 subtypes that are called IFN-α1, IFN-α2, IFN-α4, IFN-α5, IFN-α6, IFN-α7, IFN-α8, IFN-α10, IFN-α13, IFN-α14, IFN-α16, IFN-α17, and IFN-α21. These genes are found together in a cluster on chromosome 9. IFN-α is also made synthetically as a medication for hairy cell leukemia. The International Nonproprietary Name (INN) for the product is interferon alpha. The recombinant type is interferon alpha-1. The pegylated types of IFNs are pegylated interferon alpha-2a and pegylated interferon alpha-2b. The IFN-β proteins are produced in large quantities by fibroblasts. They have an antiviral activity that is involved mainly in innate immune response. Two types of IFN-β have been described, IFN-β1 (IFNB1) and IFN-β3 (IFNB3)(the gene designated IFN-β2 is actually IL-6). IFN-β1 is used as a treatment for multiple sclerosis as it reduces the relapse rate. IFN-β1 is not an appropriate treatment for patients with progressive, non-relapsing forms of multiple sclerosis. IFN-ε, -κ, -τ, -δ and -ζ, IFN-ε, -κ, -τ, and -ζ appear, at this time, to come in a single isoform in humans, IFN-κ. Ruminants encode IFN-τ, a variant of IFN-ω. So far, IFN-ζ is only found in mice, while a structural homolog, IFN-δ is found in a diverse array of non-primate and non-rodent placental mammals. Most but not all placental mammals encode functional IFN-ε and IFN-κ genes. IFN-ω, although having only one functional form described to date (IFN-ω1), has several pseudogenes: IFN-ωP2, IFN-ωP4, IFN-ωP5, IFN-ωP9, IFN-ωP15, IFN-ωP18, and IFN-ωP19 in humans. Many non-primate placental mammals express multiple IFN-ω subtypes. IFN-v, a subtype of type I IFN, was recently described as a pseudogene in humans, but potentially functional in the domestic cat genome. In all other genomes of non-feline placental mammals, IFN-v is a pseudogene; in some species, the pseudogene is well preserved, while in others, it is badly mutilated or is undetectable. Moreover, in the cat genome, the IFN-v promoter is deleteriously mutated. It is likely that the IFN-v gene family was rendered useless prior to mammalian diversification. Its presence on the edge of the type I IFN locus in mammals may have shielded it from obliteration, allowing its detection.

In embodiments, expression of the one or more therapeutic agents may be regulated by an inducible expression system. The inducible expression system allows for a temporal and spatial controlled activation and/or expression of genes. For example, Tetracycline-Controlled Transcriptional Activation is a method of inducible gene expression where transcription is reversibly turned on or off in the presence of the antibiotic tetracycline or one of its derivatives (e.g., doxycycline). For example, an inducible suicide gene expression system allows for a temporal and spatial controlled activation and/or expression of a suicide gene, which causes a cell to kill itself through apoptosis.

In embodiments, the modified cells comprise a nucleic acid sequence encoding a reverse tetracycline transactivator (rtTA). In embodiments, expression of the one or more therapeutic agents is regulated by the rtTA, such that the one or more therapeutic agents are expressed in the presence of tetracycline. In embodiments, a concentration of tetracycline in the cell culture medium is not less than about 2 μg/ml. In embodiments, the tetracycline is selected from the group consisting of tetracycline, demeclocycline, meclocycline, doxycycline, lymecycline, methacycline, minocycline, oxytetracycline, rolitetracycline, and chlortetracycline. In embodiments, the tetracycline is doxycycline.

In embodiments, the inducible suicide system is an HSV-TK system or an inducible caspase-9 system. In embodiments, the modified cells comprise a nucleic acid sequence encoding a suicide gene, such that when the modified cells are in the presence of a nucleoside analogue in a manner permitting expression of the suicide gene, to render the nucleoside analogue cytotoxic to the modified cells. In embodiments, the suicide gene is selected from the group consisting of thymidine kinase of herpes simplex virus, thymidine kinase of varicella zoster virus, and bacterial cytosine deaminase. In embodiments, the suicide gene is thymidine kinase of herpes simplex virus. In embodiments, the nucleoside analogue is selected from the group consisting of ganciclovir, acyclovir, buciclovir, famciclovir, penciclovir, valciclovir, trifluorothymidine, 1-[2-deoxy, 2-fluoro, beta-D-arabino furanosyl]-5-iodouracil, ara-A, araT 1-beta-D-arabinofuranoxyl thymine, 5-ethyl-2′-deoxyuridine, 5-iodo-5′-amino-2,5′-dideoxyuridine, idoxuridine, AZT, AIU, dideoxycytidine, and AraC. In embodiments, the nucleoside analogue is ganciclovir.

In embodiments, the CAR comprises an extracellular domain, a transmembrane domain, and an intracellular domain, and the extracellular domain binds an antigen. In embodiments, the CAR binds tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR. In embodiments, the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the therapeutic agent is present in the modified T cell in a recombinant DNA construct, in an mRNA, or in a viral vector. In embodiments, the modified T cell comprises a polynucleotide comprising a promoter comprising a binding site for a transcription modulator that modulates the expression and/or secretion of the therapeutic agent in the modified cell. In embodiments, the transcription modulator is or comprises Hif1a, NFAT, FOXP3, or NFkB. In embodiments, the promoter is responsive to the transcription modulator. In embodiments, the promoter is operably linked to the polynucleotide encoding the therapeutic agent such that the promoter drives expression and/or secretion of the therapeutic agent. In embodiments, the promoter comprises at least one of SEQ ID NOs: 323-325.

In embodiments, the CAR and the therapeutic agent are produced in the form of a polyprotein, which is cleaved to generate separate CAR and therapeutic agent molecules, and there is a cleavable moiety between the CAR and the therapeutic agent, the cleavable moiety comprises a 2A peptide, the 2A peptide comprises P2A or T2A.

In embodiments, the modified T cell comprises an additional (second) CAR, the CAR binds a solid tumor antigen, and the additional CAR binds an antigen of a white blood cell. In embodiments, the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the antigen of the white blood cell is CD19, CD20, CD22, or BCMA. In embodiments, the modified cell comprises a dominant negative form of PD-1.

Embodiments relate to an isolated nucleic acid comprising a polynucleotide and an additional polynucleotide, the nucleic acid encoding a chimeric antigen receptor (CAR), the additional nucleic acid encoding a therapeutic agent that comprises at least one of TNFRSF superfamily member receptor activation antibodies or membrane-bound forms thereof, TNFRSF superfamily member ligands or the membrane-bound form thereof, chemokines or membrane-bound forms thereof, antibodies to the chemokines, or antibodies to receptors of the chemokines or the membrane-bound forms thereof, and D28 family's ligands that correspond to the sequences in Table 2-4. For example, TNFRSF superfamily member receptor includes tumor necrosis factor receptor 1, Tumor necrosis factor receptor 2, Lymphotoxin beta receptor, Lymphotoxin beta receptor, CD40, Fas receptor, Decoy receptor 3, CD27, CD30, 4-1BB, Death receptor 4, Death receptor 5, Decoy receptor 1, Decoy receptor 2, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, and the like.

In embodiments, the therapeutic agent includes an antibody reagent (e.g., a single-chain antibody (e.g., scFv), a single domain antibody (e.g., a camelid antibody), or a bispecific antibody reagent (e.g., a bispecific T cell engager (BiTE)). In embodiments, the therapeutic agent includes a cytokine. Examples of the cytokines include IL-1P, IL-2, IL-4, IL-5, IL-6, IL-8, IL-10, IL-12, IL-13, IL-15, IL-17, IL-1Ra, IL-2R, IFN-γ, IFN-γ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, ferritin, and any combination thereof. In embodiments, the cytokines include proinflammatory cytokines such as IFN-γ, IL-15, IL-4, IL-10, TNFα, IL-8, IL-5, IL-6, GM-CSF, and MIP-1α. For example, IFN-γ has been approved by the FDA to treat patients with malignant osteoporosis (e.g., Journal of Pediatrics 121(1):119-24—August 1992).

The present disclosure describes a population of CAR cells comprising the nucleic acid and the additional nucleic acid, wherein the CAR cells comprise lymphocyte, leukocyte, or peripheral blood mononuclear cell (PBMC). In embodiments, the CAR and the therapeutic agent are produced in the form of a polyprotein, which is cleaved to generate separate CAR and therapeutic agent molecules. In embodiments, the polyprotein comprises a cleavable moiety between the CAR and the therapeutic agent, the cleavable moiety comprising a 2A peptide, the 2A peptide comprises P2A or T2A. In embodiments, the CAR and the therapeutic agent are each constitutively expressed. In embodiments, the CAR cells comprise: a third polynucleotide encoding an additional CAR binding to an antigen that is different from the CAR, or the additional CAR binding a solid tumor antigen, and the CAR binds an antigen of a white blood cell. In embodiments, the therapeutic agent or its variants can be produced either recombinantly or synthetically. For synthetic production of the therapeutic agent, an automated synthesizer can be used. For recombinant production of the therapeutic agent, a suitable plasmid containing polynucleotide that encodes the therapeutic agent can be introduced into a suitable host cell, either eukaryotic, such as yeast, plant, insect or mammalian cells, or prokaryotic, such as E. coli. Polynucleotides encoding the therapeutic agent of interest can be made by routine manipulations such as ligation of polynucleotides. The resultant therapeutic agent can be isolated using standard protein purification techniques known in the art.

The present disclosure describes a pharmaceutical composition comprising the population of the CAR cells. Embodiments relate to a method of inducing or enhancing T cell response in a subject in need thereof and/or treating a tumor of the subject, the method comprising administering an effective amount of the composition to the subject.

The present disclosure describes a modified cell comprising one or more CARs, wherein the cell is engineered to express and secrete one or more therapeutic agents comprising at least one of IL-2, IL-6, IL-7, IL-15, IL-17, and IL-23. In embodiments, the cell is engineered to express the therapeutic agent, which is bound to the membrane of the modified cell.

Modified T cells can be derived from a stem cell. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. A modified cell can also be a dendritic cell, a NK-cell, a B-cell or a T cell selected from the group consisting of inflammatory T-lymphocytes, cytotoxic T-lymphocytes, regulatory T-lymphocytes or helper T-lymphocytes. In another embodiment, Modified cells can be derived from the group consisting of CD4+ T-lymphocytes and CD8+ T-lymphocytes. Prior to expansion and genetic modification of the cells of the invention, a source of cells can be obtained from a subject through a variety of non-limiting methods. T cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, modified cells can be derived from a healthy donor, from a patient diagnosed with cancer or from a patient diagnosed with an infection. In embodiments, modified cell is part of a mixed population of cells which present different phenotypic characteristics.

The term “stem cell” refers to any of certain type of cells which have the capacity for self-renewal and the ability to differentiate into other kinds of cells. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cell may be distinguished on the basis of their origin and/or the extent of their capacity for differentiation into other types of cell. For example, stem cell may include embryonic stem (ES) cells (i.e., pluripotent stem cells), somatic stem cells, Induced pluripotent stem cells, and any other types stem cells.

The pluripotent embryonic stem cells may be found in the inner cell mass of a blastocyst and have high innate capacity for differentiation. For example, pluripotent embryonic stem cells may have the potential to form any type of cell in the body. When grown in vitro for long periods of time, ES cells maintain pluripotency: progeny cells retain the potential for multilineage differentiation.

Somatic stem cells include the fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited types of cells and have been described as multipotent. The ‘tissue-specific’ stem cells normally give rise to only one type of cell. For example, embryonic stem cells may be differentiated into blood stem cells (e.g., Hematopoietic stem cells (HSCs)), which may be further differentiated into various blood cells (e.g., red blood cells, platelets, white blood cells).

Induced pluripotent stem cells (i.e., iPS cells or iPSCs) may include a type of pluripotent stem cell artificially derived from a non-pluripotent cell (e.g., an adult somatic cell) by inducing an expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be made from adult stomach, liver, skin cells, and blood cells.

The present disclosure describes a method of causing or enhancing T cell response, treating cancer, or enhancing cancer treatment, the method comprising: administrating an effective amount of the composition of T cells comprising one or more CARs, wherein the cell is engineered to express and secrete a therapeutic agent that is or comprises at least one of IL-2, IL-6, IL-7, IL-12, IL-15, IL-17, and IL-23, and the T cell response is enhanced as compared to the administration of T cells that do not express or secrete the therapeutic agent. In embodiments, the composition of T cells may include one or more CARs and can be engineered to express and secrete IL-6 and IFNγ. In other embodiments, the T cells can be engineered to express and secrete IL-12. In other embodiments, the T cells can be engineered to express and secrete IL-15. It has been reported that patients with IL-15 administered at 0.3 mcg/kg/day were concurrent with a maximum of 50-fold elevations of circulating IL-6 and IFNγ concentrations.

The present disclosure describes a method of causing or enhancing T cell response, treating cancer, or enhancing cancer treatment, the method comprising: administering an effective amount of a composition comprising a population of T cells comprising a CAR; and administering an effective amount of a therapeutic agent comprising at least one of IFNγ, IL-2, IL-6, IL-7, IL-12, IL-15, IL-17, and IL-23, wherein the T cell response is enhanced as compared to the administration of CAR T cells without the administration of therapeutic agent. In embodiments, the therapeutic agent may be isolated, synthetic, native, or recombinant cytokines. The therapeutic agent comprises recombinant IFNγ, IL-2, IL-6, IL-7, IL-12, IL-15, IL-17, and/or IL-23. In embodiments, the recombinant cytokine is a recombinant human cytokine. In embodiments, the administering may be implemented by intravenous or subcutaneous injection. In embodiments, administering an effective amount of the therapeutic agent comprises intravenous delivery of an amount of IL-6 in the range of about 0.5-50 ug per kilogram of body weight. In embodiments, the therapeutic agent is IL-6 or IL-7. Recombinant IL-15 can be administered as a daily bolus infusion for a predetermined time or days at 3 mcg/kg/day and 1 mcg/kg/day. Recombinant IFNγ can be administered at a dose of 2 million units daily for 5 days per week over a predetermined time. In embodiments, administering the effective amount of the therapeutic agent comprises administering an effective amount of the therapeutic agent such that concentrations of the cytokines, such as IL-6 and/or IFN-γ, in the blood of the subject may increase 5-1000 times (e.g., 50 times). Methods of administering of IL-6, IL-15, and/or IFNγ can be found in U.S. Patent Application NO: U.S. Pat. No. 5,178,856A and Cytokines in the Treatment of Cancer, Volume 00, Number 00, 2018 of Journal of Interferon & Cytokine Research, which are incorporated herein by reference in their entirety. In embodiments, recombinant IL-12 can be administered at 30 ng/kg as a starting dose and escalated to 500 ng/kg twice weekly after the infusion of CAR T cells. Methods of administering of IL-12 can be found in Leuk Res. 2009 November; 33(11): 1485-1489, which is incorporated here by reference. In embodiments, the therapeutic agent can be administered to the subject starting from 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the infusion of the CAR T cells. In embodiments, the therapeutic agent can be administered to the subject between 0 and 7 days after the infusion of the CAR T cells. Sequences of examples of the recombinant cytokines can be found in Table 2.

In embodiments, the composition comprising a population of T cells described herein can comprise one or more CAR molecules. The CAR molecules can have different antigen binding domains. In embodiments, the population of T cells in the composition includes T cells comprising different CAR molecules. In embodiments, a single T cell can comprise a single CAR molecule or at least two different CAR molecules. In embodiments, a single T cell can comprise a single CAR with at least two different antigen binding domains, for example, a bispecific CAR. In embodiments, the population of T cells includes a mix of different T cells including one or more CAR molecules and/or a CAR molecule including one or more antigen binding sites.

In embodiments, the method further comprises monitoring a concentration of the therapeutic agent in tissue or blood of the subject; and administering an antagonist of receptors of the therapeutic agent or the therapeutic agent (e.g., antibodies) if the concentration and/or other parameters of the subject are not in a desired condition. For example, the parameters may include a level of body temperatures, a level CRS, and a level of neuronal toxicity.

In embodiments, the expression and/or secretion of the therapeutic agent may be is regulated by an inducible expression system. In embodiments, the inducible expression system is a rtTA-TRE system, which increases or activates the expression of the therapeutic agent, or a combination thereof. In embodiments, the inducible expression system is the rtTA-TRE system. For example, Tetracycline-Controlled Transcriptional Activation is a method of inducible gene expression where transcription is reversibly turned on or off in the presence of the antibiotic tetracycline or one of its derivatives (e.g., doxycycline). In embodiments, the expression and/or secretion of the therapeutic agent may be regulated by an inducible expression system and/or the modified cell comprises a polynucleotide encoding an inducible suicide system. For example, the inducible suicide system is an HSV-TK system, eEGFR system, or an inducible caspase-9 system.

In embodiments, the T cell comprises an additional (second) CAR binding an antigen of a WBC, and the first CAR binds an antigen of a solid tumor. In embodiments, the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the B cell antigen is CD19, CD20, CD22, or BCMA.

In embodiments, the CAR comprises an extracellular domain, a transmembrane domain, and an intracellular domain, and the extracellular domain binds an antigen.

In embodiments, the intracellular domain comprises a co-stimulatory domain that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the modified cell or T cells comprise a dominant negative (dn) PD-1 mutant to interfere with PD-1/PDL1 signaling pathway of the cell. For example, the modified cell or T cells comprise a polynucleotide encoding the dominant negative form of PD-1 mutant. In embodiments, the polynucleotide may further include a promoter comprising a binding site for a transcription modulator (e.g., transcription factors) that modulates the expression of the PD-1 mutant in the cell. Examples of the polynucleotide are provided in Tables 2-4. These constructs can be placed into vectors (e.g., lentiviral vectors) either in a forward or reverse direction. In embodiments, examples of the transcription modulator are Hif1a, NFAT, FOXP3, NFkB, and other modulators in Table 2-4.

In embodiments, the therapeutic agent is present in the modified cell in a recombinant DNA construct, in an mRNA, or in a viral vector. In embodiments, the modified cell comprises a therapeutic agent mRNA encoding the therapeutic agent, and the mRNA is not integrated into the genome of the modified cell. In embodiments, the therapeutic agent mRNA may be introduced (e.g., electroporated) into the modified cell such that the expression and/or secretion of the therapeutic agent is transient. Synthetic mRNAs can be injected to achieve transient gene expression. For example, the therapeutic agent supplied by the mRNA is short-lived such that the release of the therapeutic agent is controllable, especially for proinflammatory cytokines such as IFN-γ, IL-4, IL-10, TNFα, IL-8, IL-5, IL-6, GM-CSF, and MIP-1α.

In embodiments, the therapeutic agent comprises or is at least one listed in Table 2. In embodiments, the therapeutic agent comprises or has the sequence listed in Table 2.

In embodiments, the modified cell includes a polynucleotide comprising the isolated nucleic acids described herein, wherein the isolated nucleic acid includes a promoter comprising a binding site for a transcription modulator (e.g., transcription factors) that modulates the expression of the therapeutic agent in the cell. Examples of the isolated polynucleotide are provided in Table 2-4. These constructs may be placed into vectors (e.g., lentiviral vectors) either in a forward or reverse direction. In embodiments, the transcription modulator includes Hif1a, NFAT, FOXP3, and/or NFkB. In embodiments, the promoter is responsive to the transcription modulator. In embodiments, the promoter is operably linked to the polynucleotide encoding the therapeutic agent, such that the promoter drives the expression of the therapeutic agent in the cell. In embodiments, the therapeutic agent is ligated to a specific promoter such as to induce the expression of the therapeutic agent in a desired condition. The promoter is divided into two parts, a specific regulatory region containing a transcription factor binding site, plus a minimal promoter. In embodiments, the promoter and the binding site includes the sequences listed in Table 2-4. More information about NFAT may be found at WO2018006882, which is incorporated herein by reference. In embodiments, the transcription modulator can include Stat5 response element, (activated by cytokines such as IL2, IL3, IL7, IL15, and the transcription factor associated with it is STAT5), Stat3 response element, (activated by cytokines such as IL6, transcription factor is STAT3), Interferon Stimulated Response Element, (activated by IFN-α, transcription factors are STAT1 and STAT2), AP1 Response Element, (activated by MAPK/JNK pathway, transcription factor is AP1), SMAD Binding Element (activated by TGF-β, transcription factors are SMAD3 and SMAD4), Serum Response Element (activated by MAP/ERK pathway, transcription factor is Elk1/SRF), Serum Response Factor Response Element (activated by the RhoA pathway, the transcription factor is SRF), Cyclic AMP response element (activated by cAMP/PKA pathway, transcription factor is CREB), or TCF-LEF Response Element (activated by Wnt pathway, transcription factor is TCF-LEF).

Embodiments relate to an isolated polynucleotide comprising a (first) nucleic acid and an additional (second) polynucleotide, the first nucleic acid encoding a chimeric antigen receptor (CAR), the second nucleic acid encoding a therapeutic agent. For example, the therapeutic agent comprises IL-6 or IFN-γ, or a combination thereof. For example, the therapeutic agent comprises IL-15 or IL-12, or a combination thereof. Embodiments relate to a population of CAR cells comprising the isolated nucleic acid, wherein the CAR cells comprise lymphocyte, leukocyte, or PBMC. In embodiments, the population of CAR cells comprises the CAR and the therapeutic agent produced in the form of a polyprotein, which is cleaved to generate separate CAR and therapeutic agent molecules. In embodiments, the polyprotein comprises a cleavable moiety between the CAR and the therapeutic agent, and the cleavable moiety comprises a 2A peptide, the 2A peptide comprising P2A or T2A. In embodiments, the CAR and the therapeutic agent are each constitutively expressed. In embodiments, the CAR cells comprise: a third polynucleotide encoding a second CAR binding to an antigen that is different from the first CAR. In embodiments, the second CAR binds a solid tumor antigen, and the first CAR binds an antigen of a white blood cell.

Embodiments relate to a pharmaceutical composition comprising the population of the cells including one or more CAR molecules (the CAR cells) and one or more therapeutic agents. Embodiments also relate to a method of inducing or causing T cell response in a subject in need thereof, the method comprising administering an effective amount of the pharmaceutical composition described herein to the subject. In embodiments, the CAR cell or the modified cell is a T cell, an NK cell, a macrophage, or a dendritic cell. In embodiments, the CAR cell or the modified cell is a T cell.

In embodiments, the additional (second) nucleic acid comprises two nucleic acids, one encoding IL6, and one encoding IFN-γ, and the two nucleic acids are connected by an IRES element or another nucleic acid encoding a 2A peptide. In embodiments, the additional (second) nucleic acid comprises the polynucleotide of SEQ ID NOs: 287 or 328, or a combination thereof. In embodiments, expression of the additional (second) nucleic acid is regulated by a conditional expression system such that the therapeutic agent is expressed in response to the binding of a target antigen. In embodiments, the expression of the additional polynucleotide is regulated by SynNotch polypeptide.

Embodiments relate to an FC fusion protein associated with a small protein (e.g., a cytokine) as described herein. In embodiments, the therapeutic agent may comprise the FC fusion protein. For example, cytokines such as IL15, IFN-γ or IL6 may be linked to one or more immunoglobin Fc domains. In embodiments, the Fc domain folds independently and may improve the solubility and stability of the small protein both in vitro and in vivo. In embodiments, the Fc region allows for easy cost-effective purification by protein-G/A affinity chromatography during manufacture. In embodiments, the FC fusion protein may be modified to polymerize into well-defined complexes containing multiple small proteins. In embodiments, the fusion protein may be expressed and secreted by the modified cell (e.g., a CAR T cell), which is used to treat a subject with cancer and/or other diseases. In embodiments, administration of the fusion protein may be combined with the treatment of CAR T cells expressing and secreting the fusion protein. For example, a method for enhancing T cell response and/or treating a subject with cancer or other diseases may comprise administrating a fusion protein associated with the small protein (e.g., IFN-γ) to a subject and administrating an effective amount of the composition of a population of T cells comprising a CAR and expressing as well as secreting the fusion protein associated with the small protein to the subject. In embodiments, the administration of the fusion protein may enhance the expansion of the CAR T cells during the early stage of the CAR T treatment (e.g., 1, 2, 3, 4, 5, or 6 days after the infusion of the CAR T cells). For example, the fusion protein may be administrated into the subject 1, 2, 3, 4, 5, or 6 days after the infusion of the CAR T cells. In embodiments, the method may comprise administrating a fusion protein associated with the small protein (e.g., IFN-γ) to a subject and administrating an effective amount of the composition of a population of T cells comprising a CAR without expressing or secreting the fusion protein associated with the small protein to the subject. For example, the fusion protein may be administrated into the subject for a predetermined time. More information about the FC fusion protein may be found at J Immunol 2004; 172:2925-2934 and EMBO Mol Med. 2012 October; 4(10): 1015-1028, which are incorporated by reference. More information about administration of the therapeutic agent (e.g. cytokines) may be found at J Interferon Cytokine Res. 2019 January; 39(1):6-21, which is incorporated by reference.

Embodiments relate to a modified cell comprising one or more CARs, wherein the cell is engineered to express and secrete one or more therapeutic agents. For example, the therapeutic agent comprises IL-6 or IFN-γ, or a combination thereof. Embodiments relate to a method of inducing or enhancing T cell response, treating cancer, or enhancing cancer treatment, the method comprising: administrating an effective amount of the pharmaceutical composition of T cells comprising one or more CARs, wherein the cell is engineered to express and secrete one or more therapeutic agents. For example, the therapeutic agent comprises IL-6 or IFN-γ, or a combination thereof. In embodiments, the therapeutic agent is a small protein associated with IL-6 or IFN-γ. For example, the administration of IL-15 to a subject may increase concentrations of IL-6 and IFN-γ up to 50-fold in the blood of the patient. Embodiments relate to a method of causing or enhancing T cell response, treating cancer, or enhancing cancer treatment, the method comprising: administering an effective amount of the composition of a population of T cells comprising a CAR and administering an effective amount of a therapeutic agent. For example, a therapeutic agent comprises IL-6 or IFN-γ, or a combination thereof. In embodiments, the CAR cells, the modified cell, the cell is a T cell, an NK cell, a macrophage or a dendritic cell. For example, the CAR cells, the modified cell, the cell is a T cell. Embodiments relate to a method for enhancing T cell response and/or treating a subject with cancer, or other diseases may comprise administrating a therapeutic agent (e.g., a recombinant or native IFN-γ) to a subject and administrating an effective amount of a composition comprising a population of T cells comprising a CAR and expressing as well as secreting one or more therapeutic agents in the subject. In embodiments, the therapeutic agent may enhance the expansion of the CAR T cells during the early stage of the CAR T treatment (e.g., 1, 2, 3, 4, 5, or 6 days after the infusion of the CAR T cells). For example, the therapeutic agent may be administrated into the subject 1, 2, 3, 4, 5, or 6 days after the infusion of the CAR T cells. In embodiments, the method may comprise administrating a therapeutic agent to a subject and administrating an effective amount of the composition of a population of T cells comprising a CAR without expressing or secreting the therapeutic to the subject. For example, the therapeutic agent may be administrated into the subject for a predetermined time. In embodiments, the therapeutic agent may be modified such that the biological and/or pharmacological properties of the therapeutic agent may be enhanced. For example, the hybrid FC fusion technology may be implemented to the solubility and/or stability of an active ingredient of the therapeutic agent.

In embodiments, the therapeutic agent may be isolated, synthetic, native, or recombinant human cytokines. For example, Recombinant human IL-15 may be administered as a daily bolus infusion for predetermined time days at 3 mcg/kg/day and 1 mcg/kg/day. Recombinant human IFN-γ may be administered at a dose of 2 million units daily for 5 days per week over a predetermined time. In embodiments, the administering the effective amount of the therapeutic agent comprises administering an effective amount of the therapeutic agent such that concentrations of IL-6 and/or IFN-γ in the blood of the subject may increase 5-1000 times (e.g., 50 times). For example, the therapeutic agent comprises IL-15.

In embodiments, T cell response is enhanced as compared to the administration of T cells that do not express or secrete the therapeutic agent, or the T cell response is enhanced as compared to the administration of CAR T cells without the administration of the therapeutic agent.

In embodiments, expression and/or secretion of the therapeutic agent is regulated by an inducible expression system and/or the modified cell comprises a polynucleotide encoding an inducible suicide system. In embodiments, the inducible expression system is the rtTA-TRE system. In embodiments, the inducible suicide system is an HSV-TK system or an inducible caspase-9 system.

In embodiments, the modified T cells express and/or secrete the one or more therapeutic agents in response to the activation of the modified T cells. Such conditional expression and/or secretion may be implemented in various manners. The expression and/or secretion of the one or more therapeutic agents in the modified cell may be modulated by a transcription modulator (e.g., NFAT). TRUCKs (T cells redirected for universal cytokine killing), CAR-redirected T cells, may be used to express and/or secrete the one or more therapeutic agents when these T cells are activated. The expression and/or secretion of the one or more therapeutic agents in the modified cell may also be regulated by a SynNotch polypeptide.

In embodiments, a range of concentration values of IL6 is 60 to 5000 μg/ml, 200-5000 μg/ml, or 2000-5000 μg/ml in the blood of the subject. In embodiments, a range of concentration values IFN-γ is 20 to 5000 μg/ml, 200 to 5000 μg/ml, or 500 to 5000 μg/ml in the blood of the subject. In embodiments, the administering an effective amount of the therapeutic agent comprises intravenous delivery of an amount of human IL-6 in the range of about 0.5-50 ug per kilogram of body weight. More detailed information about IFN-γ clinical uses may be found at Cancer Med. 2018, 7: 4509-4516, which is incorporated by reference.

In embodiments, the modified cell expresses the therapeutic agent such that concentrations of IL-6 and/or IFN-γ in the blood of the subject may increase 5-1000 times (e.g., 50 times). For example, the therapeutic agent comprises IL-15. It was reported that transgenic expression of IL15 improved anti-glioma activity of anti-IL13Rα2 CAR T cells but cause loss variants such that expression of IL13Rα2 expression was downregulated. As a result, gliomas recurred after the initial response of anti-IL13Rα2 CAR T cells. In embodiments, the modified cells comprise a first group of modified cells engineered to express anti-CD19 CAR and IL-15 and a second group of modified cells engineered to express CAR binding a solid tumor antigen. A pharmaceutical composition comprising the modified cells may be administered to a subject to treat the subject's solid tumor. In this instance, anti-CA19 CAR cells (e.g., T cells) are used to enhance activation/expansion of anti-solid tumor antigen CAR cells (e.g., T cells) at least at the early stage of the treatment. Downregulation of CD19 antigen may have minimal or little impact on treatment of the solid tumor using the anti-solid tumor antigen CAR cells. Thus, a combination of expression of IL-15 and the pharmaceutical composition comprising multiple types of modified cells may improve immunotherapy as compared to conventional treatment of CAR T cells expressing IL-15. In embodiments, IL-15 expression by the anti-CA19 CAR can be regulated by various methods described in the present disclosure.

In embodiments, the modified cells or the T cells comprise an additional (second) CAR binding an antigen of a WBC, and the CAR binds an antigen of a solid tumor. In embodiments, the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the B cell antigen is CD19, CD20, CD22, or BCMA.

In embodiments, the modified cells or the T cells comprise a dominant negative form of PD-1. In embodiments, the modified cell or the T cells comprise a modified PD-1 lacking a functional PD-1 intracellular domain.

In embodiments, the CAR comprises an extracellular domain, a transmembrane domain, and an intracellular domain, and the extracellular domain binds an antigen. In embodiments, the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and one combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the therapeutic agent is present in the modified cell in a recombinant DNA construct, in an mRNA, or in a viral vector. In embodiments, the modified cell comprises a therapeutic agent mRNA encoding the therapeutic agent, and the mRNA is not integrated into the genome of the modified cell. In embodiments, the modified cell comprises a polynucleotide comprising a promoter which comprises a binding site for a transcription modulator that modulates the expression and/or secretion of the therapeutic agent in the cell. In embodiments, the transcription modulator includes Hif1a, NFAT, FOXP3, and/or NFkB. In embodiments, the promoter is responsive to the transcription modulator. In embodiments, the promoter is operably linked to the polynucleotide encoding the therapeutic agent such that the promoter drives expression and/or secretion of the therapeutic agent in the cell. In embodiments, the promoter comprises at least one of SEQ ID NOs: 323-325.

In embodiments, the CAR cells, the modified cell, the cell is a T cell, an NK cell, a macrophage, or a dendritic cell. For example, the CAR cells, the modified cell, the cell is a T cell.

In embodiments, the population of cells described herein is used in autologous CAR T cell therapy. In embodiments, the CAR T cell therapy is allogeneic CAR T cell therapy, TCR T cell therapy, and NK cell therapy.

### CAR Molecules

In addition to the embodiments described herein, the present disclosure describes isolated nucleic acids encoding at least two different antigen binding domains. In embodiments, there is a first antigen binding domain that binds an antigen on the surface of a WBC, and there is a second antigen binding domain that binds an antigen on a tumor that is different from the antigen on the surface of a WBC. The first antigen binding domain functions to expand the cells that it is introduced into, while the second antigen binding domain functions to inhibit the growth of or kill tumor cells containing the target tumor antigen upon binding to the target antigen. In embodiments, an isolated nucleic acid described herein encodes both the first and second antigen binding domains on the same nucleic acid molecule. In embodiments, the two antigen binding domains are encoded by two separate nucleic acid molecules. For example, a first nucleic acid encodes a first antigen binding domain and a second nucleic acid encodes a second antigen binding domain.

In embodiments, the present disclosure describes nucleic acids encoding a first antigen binding domain of a binding molecule and a second antigen binding domain of a binding molecule, wherein first antigen binding domain binds a cell surface molecule of a WBC, and the second antigen binding domain binds an antigen different from the cell surface molecule of the WBC. In embodiments, the second binding domain does not bind a B cell marker. In embodiments, the second binding domain includes a ScFv comprising an amino acid sequence of SEQ ID No: 264 or 265. For example, the second antigen binding domain is on a CAR having one of the amino acid sequences of SEQ ID NOs: 271-277.

In embodiments, the first and second antigen binding domains can be on two different binding molecules (first and second binding molecules) such as a first CAR and a second CAR. As an example, a first CAR includes an extracellular binding domain that binds a marker on the surface of a B cell, and a second CAR includes an extracellular binding domain that binds a target antigen of a tumor cell. In embodiments, the first CAR and second CAR are encoded by different nucleic acids. In embodiments, the first CAR and second CAR are two different binding molecules but are encoded by a single nucleic acid.

In embodiments, the two different antigen binding domains can be on the same binding molecule, for example on a bispecific CAR, and encoded by a single nucleic acid. In embodiments, the bispecific CAR can have two different scFv molecules joined together by linkers.

In embodiments, the two different antigen binding domains can be on a CAR and a T cell receptor (TCR) and are encoded by separate nucleic acids. The binding domain of a TCR can target a specific tumor antigen or tumor marker on the cell of a tumor. In embodiments, the TCR binding domain is a TCR alpha binding domain or TCR beta binding domain that targets a specific tumor antigen. In embodiments, the TCR comprises the TCRγ and TCRδ chains or the TCRα and TCRβ chains. The present disclosure also describes vectors including the isolated nucleic acids described herein. In embodiments, a single vector contains the isolated nucleic acid encoding the first CAR and second CAR or TCR. In embodiments, a first vector contains the first nucleic acid encoding a first CAR, and a second vector contains the nucleic acid encoding the second CAR or TCR. In embodiments, the vector comprises a bispecific CAR including at least two antigen binding domains.

Moreover, the present disclosure describes cells comprising the isolated nucleic acids or vectors described herein. The cells have been introduced with the isolated nucleic acids or vectors described herein and express at least two more binding domains. In embodiments, the cells include two or more different binding domains, a first antigen binding domain, and a second antigen binding domain, wherein the first antigen binding domain binds a cell surface molecule of a WBC, and the second antigen binding domain binds an antigen different from the cell surface molecule of a WBC. Further, the present disclosure describes compositions including a population of the cells described herein. In embodiments, the cells are peripheral blood mononuclear cells (PBMCs) such as lymphocytes. In embodiments, the lymphocytes are T cells, NK cells, or dendritic cells.

The present disclosure also describes methods of culturing cells described herein. The methods described herein include obtaining a cell comprising a first antigen binding domain and a second antigen binding domain, wherein the first antigen binding domain binds a cell surface molecule of a WBC, and the second antigen binding domain binds an antigen different from the cell surface molecule of the WBC; and culturing the cell in the presence of an agent derived from a cell surface molecule of the WBC or from an antigen to which the second antigen binding domain binds. In embodiments, the agent is an extracellular domain of a cell surface molecule of a WBC.

The present disclosure describes methods for in vitro cell preparation, wherein the method includes providing cells; introducing one or more nucleic acids encoding a first antigen binding domain and a second antigen binding domain into the cells, wherein the first antigen binding domain binds a cell surface molecule of a WBC, and the second antigen binding domain binds an antigen different from the cell surface molecule of the WBC; and culturing the cells in the presence of an agent derived from the cell surface molecule of the WBC or from an antigen to which the second antigen binding domain binds.

The present disclosure describes using the prepared cells to enhance T cell expansion in a subject having cancer. In embodiments, the method comprises introducing a plurality of nucleic acids into T cells, the plurality of nucleic acids encoding a chimeric antigen receptor (CAR) binding a solid tumor antigen and encoding a CAR binding a B cell antigen, at least a portion of the T cells comprising the CAR binding the solid tumor antigen and the CAR binding the B cell antigen; and administering an effective amount of the T cells to the subject. The T cell expansion is enhanced, or the number of T cells is increased in the subject as compared to a subject that is administered with T cells comprising the plurality of nucleic acids encoding only one CAR.

Additionally, the present disclosure describes methods for introducing and/or enhancing lymphocyte (T cell) response in a subject. Embodiments described herein involve a mechanism that expands lymphocytes and a mechanism that relates to binding of an antigen on a CAR to a tumor cell. In embodiments, the first mechanism involves a molecule associated with a signal that is involved in expanding the lymphocytes in a subject, and an additional mechanism involves a molecule associated with a signal directed to binding, inhibiting the growth of, or killing a tumor cell in the subject. For example, the first mechanism includes a CAR binding to an antigen associated with blood, such as blood cells and blood plasma, or non-essential tissues, and the additional mechanism includes a CAR or TCR targeting an antigen associated with the tumor cell. Examples of non-essential tissues include the mammary gland, colon, gastric gland, ovary, blood components, such as WBC, and thyroid. In embodiments, the first mechanism involves a first binding domain of a molecule, and the additional mechanism involves a second domain of a molecule. In embodiments, the first mechanism and the additional mechanism are performed by the same molecule or by separate molecules. In particular embodiments, the mechanism involves a cell expressing an antigen associated with a tumor cell, and the additional mechanism involves a lymphocyte having an antigen binding domain.

The methods described herein involve lymphocytes including an expansion molecule and a functional molecule. In embodiments, the expansion molecule expands the lymphocytes in a subject, and/or the function molecule inhibits the growth of or kills a tumor cell in the subject. In embodiments, the expansion molecule and the function molecule are on a single CAR molecule, for example, a bispecific CAR molecule. In embodiments, the expansion molecule and the function molecule are on separate molecules, for example, CAR and TCR or two different CARs. The expansion molecule can include a CAR binding to an antigen associated with blood (e.g., blood cells and blood plasma) or non-essential tissues, and the function molecule can include a CAR or TCR targeting an antigen associated with the tumor cell.

Lymphocyte or T cell response in a subject refers to cell-mediated immunity associated with a helper, killer, regulatory, and other types of T cells. For example, T cell response may include activities such as assisting other WBCs in immunologic processes and identifying and destroying virus-infected cells and tumor cells. T cell response in the subject can be measured via various indicators such as a number of virus-infected cells and/or tumor cells that T cells kill, the amount of cytokine that T cells release in co-culturing with virus-infected cells and/or tumor cells, a level of proliferation of T cells in the subject, a phenotype change of T cells, for example, changes to memory T cells, and a level longevity or lifetime of T cells in the subject.

In embodiments, the method of enhancing T cell response comprises treating a subject in need thereof, for example, a subject diagnosed with a tumor. The term tumor refers to a mass, which can be a collection of fluid, such as blood, or a solid mass. A tumor can be malignant (cancerous) or benign. Examples of blood cancers include chronic lymphocytic leukemia, acute myeloid leukemia, acute lymphoblastic leukemia, and multiple myeloma.

Solid tumors usually do not contain cysts or liquid areas. The major types of malignant solid tumors include sarcomas and carcinomas. Sarcomas are tumors that develop in soft tissue cells called mesenchymal cells, which can be found in blood vessels, bone, fat tissues, ligament lymph vessels, nerves, cartilage, muscle, ligaments, or tendon, while carcinomas are tumors that form in epithelial cells, which are found in the skin and mucous membranes. The most common types of sarcomas include undifferentiated pleomorphic sarcoma which involves soft tissue and bone cells; leiomyosarcoma which involves smooth muscle cells that line blood vessels, gastrointestinal tract, and uterus; osteosarcoma which involves bone cells, and liposarcoma which involves fat cells. Some examples of sarcomas include Ewing sarcoma, Rhabdomyosarcoma, chondosarcoma, mesothelioma, fibrosarcoma, fibrosarcoma, and glioma.

The five most common carcinomas include adrenocarcinoma which involves organs that produce fluids or mucous, such as the breasts and prostate; basal cell carcinoma which involves cells of the outer-most layer of the skin, for example, skin cancer; squamous cell carcinoma which involves the basal cells of the skin; and transitional cell carcinoma which affects transitional cells in the urinary tract which includes the bladder, kidneys, and ureter. Examples of carcinomas include cancers of the thyroid, breast, prostate, lung, intestine, skin, pancreas, liver, kidneys, and bladder, and cholangiocarcinoma.

The methods described herein can be used to treat a subject diagnosed with cancer. Cancer can be a blood cancer or can be a solid tumor, such as a sarcoma or carcinoma. The method of treating includes administering an effective amount of T cells comprising a first antigen binding domain and a second antigen binding domain to the subject to provide a T-cell response, wherein the first antigen binding domain binds a cell surface molecule of a WBC, and the second antigen binding domain binds an antigen different from the cell surface molecule of the WBC. In embodiments, enhancing the T cell response in the subject includes selectively enhancing proliferation of T cell expressing the first antigen binding domain and the second antigen binding domain in vivo.

In embodiments, the T cells for enhancing T cell response in a subject includes administering to the subject, T cells comprising a bispecific CAR including two different binding domains or administering T cells comprising a first CAR and a second CAR, wherein the first CAR and the second CAR, each includes a different antigen binding domain.

In embodiments, methods for enhancing T cell response in a subject include administering a T cell including a CAR molecule and a TCR molecule. The CAR molecule targets or binds a surface marker of a white blood cell, and the TCR molecule binds a marker or an antigen of the tumor that is expressed on the surface or inside the tumor cell.

The present disclosure describes methods of expanding cells expressing an antigen binding domain in vivo. The method includes administering an effective amount of T cells comprising a first antigen binding domain and a second antigen binding domain to a subject in need thereof, wherein the first antigen binding domain binds a cell surface molecule of a WBC, and the second antigen binding domain binds an antigen different from the cell surface molecule of the WBC. The methods are useful for expanding or increasing the number of T cells, NK cells, dendritic cells.

In embodiments, the first antigen binding domain is on a first chimeric antigen receptor (CAR) and the second antigen binding domain is on a second CAR or a TCR. For example, the first CAR and the second CAR or TCR include an extracellular antigen binding domain, a transmembrane domain, and a cytoplasmic domain. The cytoplasmic domain of the first CAR includes a co-stimulatory domain and a CD3 zeta domain for transmitting signals for activation of cellular responses. In embodiments, the cytoplasmic domain of the first CAR includes one or more co-stimulatory domains in the absence of a CD3 zeta domain such that activation or stimulation of the first CAR expands WBCs, such as lymphocytes, without introducing and/or activating the killing function of the WBCs. In embodiments, the lymphocytes are T cells.

In embodiments, the first and second antigen binding domains are on the same CAR (the first CAR), for example, a bispecific CAR with an extracellular antigen binding domain, a transmembrane domain, and a cytoplasmic domain. The extracellular antigen binding domain includes at least two scFvs and at least one of the scFvs functions as a first antigen binding domain for binding a cell surface molecule of a WBC.

In embodiments, the antigen different from the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, CD13, B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2.

In embodiments, the MUC1 is a tumor-exclusive epitope of a human MUC1, and the first CAR and the second CAR or the TCR are expressed as separate polypeptides. In embodiments, the MUC1 is a tumor form of human MUC1 (tMUC1).

In embodiments, the first CAR includes a co-stimulatory domain without a signaling domain, such as the CD3 zeta domain, and the MUC1 CAR (second CAR) comprises the MUC1 binding domain, a transmembrane domain, a co-stimulatory, and a CD3 zeta domain.

As used herein, the term “MUC1” refers to a molecule defined as follows. MUC1 is one of the epithelial mucin family of molecules. MUC1 is a transmembrane mucin glycoprotein that is normally expressed in all glandular epithelial cells of the major organs. In normal cells, MUC1 is only expressed on the apical surface and is heavily glycosylated with its core proteins sequestered by the carbohydrates. As cells transform into a malignant phenotype, expression of MUC1 increases several folds, and the expression is no longer restricted to the apical surface, but it is found all around the cell surface and in the cytoplasm. In addition, the glycosylation of tumor-associated MUC1 is aberrant, with greater exposure of the peptide core than is found on MUC1 expressed in normal tissues. Little is known regarding the specifics of the aberrant glycosylation.

MUC1 is widely expressed on a large number of epithelial cancers and is aberrantly glycosylated making it structurally and antigenically distinct from that expressed by non-malignant cells (see, e.g., Barratt-Boyes, 1996; Price et al., 1998; Peterson et al., 1991). The dominant form of MUC1 is a high molecular weight molecule comprising a large highly immunogenic extracellular mucin-like domain with a large number of twenty amino acid tandem repeats, a transmembrane region, and a cytoplasmic tail (Quin et al., 2000; McGucken et al., 1995; Dong et al., 1997).

In most epithelial adenocarcinomas including breast and pancreas, MUC1 is overexpressed and aberrantly glycosylated. Adenocarcinoma of the breast and pancreas not only overexpress MUC1 but also shed MUC1 into the circulation. High MUC1 serum levels are associated with progressive disease. MUC1 has been exploited as a prospective biomarker because of the complex and heterogeneous nature of the epitopes expressed within the antigen. MUC1 synthesized by cancerous tissues (e.g., tumor-associated MUC1) usually displays an aberrant oligosaccharide profile, which gives rise to the expression of neomarkers such as sialyl-Lea (assayed in the CA19-9 test), sialyl-Lex, and sialyl-Tn (TAG-72), as well as the cryptic epitopes such as Tn.

Several antibodies are being developed against MUC1 for therapeutic use. Pemtumomab (also known as HMFG1) is in Phase III clinical trials as a carrier to deliver the radioisotope Yttrium-90 into tumors in ovarian cancer (reviewed in Scott et al., 2012). CA15-3 (also the HMFG1 antibody), CA27-29, and CA19-9 are all antibodies to MUC1 that are used to assess levels of circulating MUC1 in patients with cancer. However, these antibodies have shown limited utility as therapeutic agents or as biomarkers because they cannot distinguish effectively between MUC1 expressed in normal versus transformed tumor epithelia. In other words, none of these antibodies appear to be targeted to a tumor-specific MUC1 epitope.

A new antibody that is highly specific for a tumor-specific form of MUC1 (tMUC) is designated TAB-004 and is described in U.S. Pat. No. 8,518,405 (see also Curry et al., 2013). While Pemtumomab (HMFG1) was developed using human milk fat globules as the antigen (Parham et al., 1988), TAB-004 was developed using tumors expressing an altered form of MUC1 (Tinder et al., 2008). TAB-004 recognizes the altered glycosylated epitope within the MUC1 tandem repeat sequence. This area is accessible for antigenic detection in tMUC but is blocked from antigenic detection in normal MUC1 by large branches of glycosylation (Gendler, 2001; Mukherjee et al., 2003b; Hollingsworth & Swanson, 2004; Kufe, 2009). Importantly, TAB-004 is different from the epitopes recognized by other MUC1 antibody and has unique complementary determinant regions (CDRs) of the heavy and light chains. The antibody binds the target antigen with a high binding affinity at 3 ng/ml (20 pM) and does not bind unrelated antigens (Curry et al., 2013). Thus, TAB-004 distinguishes between normal and tumor form of MUC1 while HMFG1 (Pemtumomab) does not (see U.S. Pat. No. 8,518,405).

In embodiments, the WBC is a granulocyte, monocyte, and/or lymphocyte. In embodiments, the WBC is a B cell.

In embodiments, the first CAR comprises the first antigen binding domain, a transmembrane domain, a co-stimulatory domain, and a CD3 zeta domain and/or the second CAR comprises the second antigen binding domain, a transmembrane domain, a co-stimulatory domain, and a CD3 zeta domain.

In embodiments, the antigen binding domain is a Fab or a scFv. In embodiments, the first CAR comprises the amino acid sequence of one of SEQ ID NO: 5, 6, and 53-58; and the second CAR comprises the amino acid sequence of one of SEQ ID NOs: 5-17, 29, 33, 37, 71, and 72, or the amino acid sequence encoded by the polynucleotide of one of SEQ ID NOs: 41, 45, 63, 67, and 68. In embodiments, a polynucleotide encoding the first CAR comprises the polynucleotide of SEQ ID NO: 59 or 60, and a polynucleotide encoding the second CAR comprises the polynucleotide of SEQ ID NO: 61. In embodiments, the isolated nucleic acid comprises one of the polynucleotides of SEQ ID NO: 62-69. In embodiments, the first CAR and the second CAR are expressed as separate polypeptides.

In embodiments, the first antigen binding domain is on a CAR, and the second antigen binding domain is on a T Cell Receptor (TCR). In embodiments, the TCR is a modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1.

In embodiments, a T cell clone that expresses a TCR with a high affinity for the target antigen may be isolated. Tumor-infiltrating lymphocytes (TILs) or PBMCs can be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle, for example, a gammaretrovirus or lentivirus, can then be generated and tested for functionality (antigen specificity and functional avidity) and used to produce a clinical lot of the vector. An aliquot of the final product can then be used to transduce the target T cell population (generally purified from patient PBMCs), which is expanded before infusion into the patient.

Various methods may be implemented to obtain genes encoding tumor-reactive TCR. More information is provided in Kershaw et al., Clin Transl Immunology. 2014 May; 3(5): e16. In embodiments, specific TCR can be derived from spontaneously occurring tumor-specific T cells in patients. Antigens included in this category include the melanocyte differentiation antigens MART-1 and gp100, as well as the MAGE antigens and NY-ESO-1, with expression in a broader range of cancers. TCRs specific for viral-associated malignancies can also be isolated, as long as viral proteins are expressed by transformed cells. Malignancies in this category include liver and cervical cancer, associated with hepatitis and papilloma viruses, and Epstein-Barr virus-associated malignancies. In embodiments, target antigens of the TCR may include CEA (e.g., for colorectal cancer), gp100, MART-1, p53 (e.g., for Melanoma), MAGE-A3 (e.g., Melanoma, esophageal and synovial sarcoma), NY-ESO-1 (e.g., for Melanoma and sarcoma as well as Multiple myelomas).

In embodiments, preparation and transfusion of tumor infiltrating lymphocytes (TIL) may be implemented by the following. For example, tumor tissue from surgical or biopsy specimens, can be obtained under aseptic conditions and transported to the cell culture chamber in ice box. Necrotic tissue and adipose tissue can be removed. The tumor tissue may be cut into small pieces of about 1-3 cubic millimeter. Collagenase, hyaluronidase, and DNA enzyme can be added, and digested overnight at 4° C. Filtering with 0.2 um filter, cells can be separated and collected by lymphocyte separation fluid, 1500 rpm for 5 min. Expanding the cells with a culture medium comprising PHA, 2-mercaptoethanol, and CD3 monoclonal antibody, a small dose of IL-2 (10-20 IU/ml) can be added to induce activation and proliferation. According to the growth situation, the cell density can be carefully detected and maintained within the range of 0.5-2×10⁶/ml under the condition of 37° C. and 5% CO2 for 7-14 days. TIL positive cells have the ability to kill homologous cancer cell may be screened out by co-culture. The positive cells can be amplified in a serum-free medium containing a high dose of IL2 (5000-6000 IU/ml) until greater than 1×10¹¹ TILs can be obtained. To administer TILs, they may be first collected in saline solution using continuous-flow centrifugation and then filtered through a platelet-administration set into a volume of 200-300 mL containing 5% albumin and 450 000 IU of IL-2. The TILs may be infused into patients through a central venous catheter over a period of 30-60 minutes. In embodiments, TILs may be infused in two to four separate bags; the infusions may be separated by several hours.

In embodiments, a binding domain of the first CAR binds CD19, and a binding domain of the second CAR binds tumor-associated MUC1. In embodiments, the binding domain of the second CAR comprises: (i) a heavy chain complementary determining region 1 comprising the amino acid sequence of SEQ ID: 76 or 85, a heavy chain complementary determining region 2 comprising the amino acid sequence of SEQ ID: 77 or 86, and a heavy chain complementary determining region 3 comprising the amino acid sequence of SEQ ID: 78 or 87; and (ii) a light chain complementary determining region 1 comprising the amino acid sequence of SEQ ID: 73 or 82, a light chain complementary determining region 2 comprising the amino acid sequence of TRP-ALA-SER (WAS) or SEQ ID: 83, and a light chain complementary determining region 3 comprising the amino acid sequence of SEQ ID: 75 or 84.

In embodiments, the binding domain of the second CAR comprises: (i) a heavy chain complementary determining region 1 comprising the amino acid sequence of SEQ ID: 76, a heavy chain complementary determining region 2 comprising the amino acid sequence of SEQ ID: 77, and a heavy chain complementary determining region 3 comprising the amino acid sequence of SEQ ID: 78; and (ii) a light chain complementary determining region 1 comprising the amino acid sequence of SEQ ID: 73, a light chain complementary determining region 2 comprising the amino acid sequence of TRP-ALA-SER (WAS), and a light chain complementary determining region 3 comprising the amino acid sequence of SEQ ID: 75.

In embodiments, the binding domain of the second CAR comprises: (i) a heavy chain complementary determining region 1 comprising the amino acid sequence of SEQ ID: 85, a heavy chain complementary determining region 2 comprising the amino acid sequence of SEQ ID: 86, and a heavy chain complementary determining region 3 comprising the amino acid sequence of SEQ ID: 87; and (ii) a light chain complementary determining region 1 comprising the amino acid sequence of SEQ ID: 82, a light chain complementary determining region 2 comprising the amino acid sequence of SEQ ID: 83, and a light chain complementary determining region 3 comprising the amino acid sequence of SEQ ID: 84. In embodiments, the binding domain of the first CAR comprises the amino acid sequence of SEQ ID: 5 or 6. In embodiments, the binding domain of the second CAR comprises one of the amino acid sequences of SEQ ID: 70-72 and 79-81.

In embodiments, the first CAR comprises the first antigen binding domain, a transmembrane domain, a co-stimulatory domain, and a CD3 zeta domain and/or the second CAR comprises the second antigen binding domain, a transmembrane domain, a co-stimulatory domain, and a CD3 zeta domain.

In embodiments, the first CAR and the second CAR are expressed as separate polypeptides.

In embodiments, the cytoplasmic domain or the transmembrane domain of the second CAR is modified such that the second CAR is capable of activating the modified T cell via cells expressing CD19 without damaging the cells expressing CD19.

Embodiments described herein relate to a bispecific chimeric antigen receptor, comprising: a first antigen binding domain, a second antigen binding domain, a cytoplasmic domain, and transmembrane domain, wherein the first antigen binding domain recognizes a first antigen, and the second antigen binding domain recognizes a second antigen, the first antigen is different from the second antigen.

In embodiments, the first antigen and the second antigen do not express on the same cell. In embodiments, the first antigen is an antigen of a blood component, and the second antigen is an antigen of a solid tumor.

Blood cells refer to red blood cells (RBCs), white blood cells (WBCs), platelets, or other blood cells. For example, RBCs are blood cells of delivering oxygen (O₂) to the body tissues via the blood flow through the circulatory system. Platelets are cells that are involved in hemostasis, leading to the formation of blood clots. WBCs are cells of the immune system involved in defending the body against both infectious disease and foreign materials. There are a number of different types and sub-types of WBCs, and each has a different role to play. For example, granulocytes, monocytes, and lymphocytes are 3 major types of white blood cells. There are three different forms of granulocytes: Neutrophils, Eosinophils, Basophils.

A cell surface molecule of a WBC refers to a molecule expressed on the surface of the WBC. For example, the cell surface molecule of a lymphocyte may include CD19, CD22, CD20, BCMA, CD5, CD7, CD2, CD16, CD56, and CD30. The cell surface molecule of a B cell may include CD19, CD20, CD22, BCMA. The cell surface molecule of a monocyte may include CD14, CD68, CD11b, CD18, CD169, and CD1c. The cell surface molecule of granulocyte may include CD33, CD38, CD138, and CD13.

In embodiments, the first antigen is CD19, and the second antigen is a tumor-associated MUC1. In embodiments, the first antigen binding domain comprises one of the amino acid sequences of SEQ ID: 5 and 6. In embodiments, the second antigen binding domain comprises one of the amino acid sequence of SEQ ID: 70-72 and 79-81.

In embodiments, the present disclosure describes a method of enhancing T cell response in a subject or treating a tumor of the subject, the method comprising: administering an effective amount of modified T cell to the subject to provide a T cell response such that the CAR T cell is expanded in the blood of the subject via cells expressing CD19.

In embodiments, the tumor-associated MUC1 is expressed on tumor cells, but not on corresponding non-malignant cells. In embodiments, a scFv against the tumor-associated MUC1 directly interacts with an o-glycosylated GSTA motif (SEQ ID NO. 88).

Embodiments described herein relate to a cell comprising the bispecific CAR and to an isolated nucleic acid encoding the bispecific CAR.

In embodiments, the present disclosure describes a method of in vivo cell expansion. In embodiments, the method may include administering an effective amount of T cell comprising a CAR to the subject to provide a T cell response; and administering an effective amount of presenting cells (e.g., T cells) expressing a soluble agent that an extracellular domain of the CAR recognizes. In embodiments, the method may be implemented to enhance T cell response in a subject. The method may include administering an effective amount of T cell comprising a CAR to the subject to provide a T cell response and administering an effective amount of presenting cells expressing a soluble agent that an extracellular domain of the CAR recognizes to enhance the T cell response in the subject. In certain embodiments, the presenting cells are T cells, dendritic cells, and/or antigen-presenting cells. In certain embodiments, the enhancing T cell response in the subject may include selectively enhancing proliferation of T cell comprising the CAR. In embodiments, the method may be used to enhance the treatment of a condition on a subject using CAR T cells. The method may include administering a population of cells that express an agent or administering an agent that is formulated as a vaccine. In these instances, the CAR T cells include a nucleic acid that encodes a CAR, and an extracellular domain of the CAR recognizes the agent. In embodiments, the method may be implemented to enhance the proliferation of CAR T cells in a subject having a disease. The method may include preparing CAR T cells comprising a CAR; administering an effective amount of the CAR T cells to the subject; introducing, into cells, a nucleic acid encoding an agent that an extracellular domain of the CAR recognizes; and administering an effective amount of the cells (introduced with the nucleic acid encoding the agent) to the subject. In embodiments, the T cell expansion or increase in the number of T cells may be measured based on an increase in copy number of CAR molecules in genomic DNA of the T cells. In embodiments, the T cell expansion or increase in the number of T cells may be measured based on flow cytometry analysis on molecules expressed on the T cells.

Embodiments described herein relate to an isolated T cell comprising a first CAR, and a second CAR, wherein an antigen binding domain of the first CAR binds an antigen such as CD19, CD33, CD14, and BCMA, and an antigen binding domain of the second CAR binds a tumor-associated MUC. In embodiments, the tumor-associated MUC is MUC1 or MUC2. Embodiments described herein relate to a composition comprising a population of the isolated T cells and to a method of enhancing T cell response in a subject or treating a tumor of the subject, the method comprising: administering an effective amount of the isolated T cell.

In embodiments, the first CAR comprises the amino acid sequence of SEQ ID NO: 207, and the second CAR comprises the amino acid sequence of SEQ ID: 202. In embodiments, the first CAR comprises the amino acid sequence of SEQ ID NO: 203, 207, 216, or 219, and the second CAR comprises the amino acid sequence of SEQ ID: 202 or 205. In embodiments, the antigen binding domain of the second CAR comprises the amino acid sequence of SEQ ID NO: 70. In embodiments, the antigen binding domain of the second CAR comprises the amino acid sequence of SEQ ID NO: 5 or 6. In embodiments, the isolated T cell comprises a polynucleotide of SEQ ID NO: 201, 204, 206, 208, 215, 217, 218, or 220. In embodiments, each of the first CAR and the second CAR comprises an antigen binding domain, a transmembrane domain, and a cytoplasmic domain.

In embodiments, the cytoplasmic domain comprises a co-stimulatory domain and a CD3 zeta domain.

In embodiments, the isolated T cell comprises a dominant negative variant of a receptor of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), or CD 160. In embodiments, the isolated T cell comprises a reduced amount of TCR, as compared to the corresponding wide-type T cell. Dominant negative mutations have an altered gene product that acts antagonistically to the wild-type allele. These mutations usually result in an altered molecular function (often inactive) and are characterized by a dominant or semi-dominant phenotype.

The present disclosure describes pharmaceutical compositions. The pharmaceutical compositions include one or more of the following: CAR molecules, TCR molecules, modified CAR T cells, modified cells comprising CAR or TCR, modified cells, nucleic acids, and vectors described herein. Pharmaceutical compositions are administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In certain embodiments, it may be desired to administer activated T cells to a subject and then subsequently redraw the blood (or have apheresis performed), collect the activated and expanded T cells, and reinfuse the patient with these activated and expanded T cells. This process can be carried out multiple times every few weeks. In certain embodiments, T cells can be activated from blood draws from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, may select out certain populations of T cells.

The administration of the pharmaceutical compositions described herein may be carried out in any convenient manner, including by aerosol inhalation, injection, ingestion, transfusion, implantation, or transplantation. The compositions described herein may be administered to a patient subcutaneously, intradermally, intratumorally, intranodally, intramedullary, intramuscularly, by intravenous (i. v.) injection, or intraperitoneally. In embodiments, the T cell compositions described herein are administered to subjects by intradermal or subcutaneous injection. In embodiments, the T cell compositions of the present disclosure are administered by i.v. injection. The compositions of T cells may be injected directly into a tumor, lymph node, or site of infection. In embodiments, cells activated and expanded using the methods described herein, or other methods known in the art where T cells are expanded to therapeutic levels, are administered to patients in conjunction with (e.g., before, simultaneously or following) any number of relevant treatment modalities, including but not limited to treatment with agents for antiviral therapy, cidofovir and interleukin-2, Cytarabine (also known as ARA-C); or natalizumab treatment for MS patients; or efalizumab treatment for psoriasis patients or other treatments for PML patients. In further embodiments, the T cells described herein can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions described herein are administered to a subject in conjunction with (e.g., before, simultaneously or following) bone marrow transplantation, T cell ablative therapy using either chemotherapy agents such as fludarabine, external-beam radiation therapy (XRT), cyclophosphamide, or antibodies such as OKT3 or CAMPATH. In embodiments, the cell compositions described herein are administered following B-cell ablative therapy. For example, agents that react with CD20, e.g., Rituxan may be administered to patients. In embodiments, subjects may undergo standard treatment with high dose chemotherapy followed by peripheral blood stem cell transplantation. In certain embodiments, following the transplant, subjects receive an infusion of the expanded immune cells of the present disclosure. In embodiments, expanded cells are administered before or following surgery.

In embodiments, CpG oligonucleotides (e.g., Class B CpG oligonucleotides) can be systemically and repeatedly administered to a subject to enhance anti-tumor effect of the pharmaceuticals described herein (e.g., CAR T cells) by introducing macrophage activation. For example, administration of CAR T cells and CpG oligonucleotides can be combined to treat a subject having a solid tumor. Information on administration of CpG oligonucleotides may be found at Nat Immunol. 2019 March; 20(3): 265-275, which is incorporated by reference in its entirety.

The dosage of the above treatments to be administered to a subject in need thereof will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

Embodiments described herein relate to an in vitro method for preparing modified cells. The method may include obtaining a sample of cells from a subject. For example, the sample may include T cells or T cell progenitors. The method may further include transfecting the sample of cells with a DNA encoding at least a CAR and culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

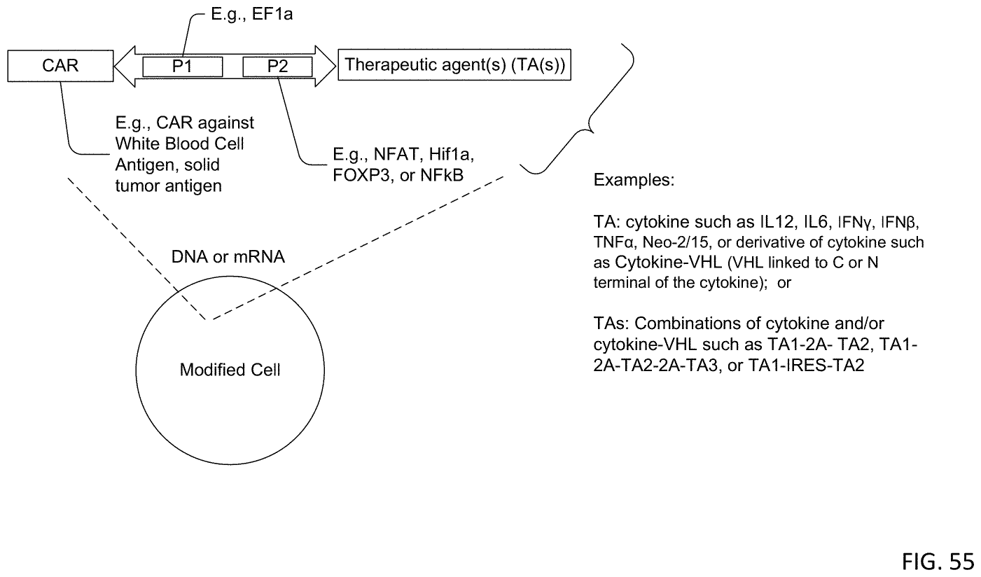

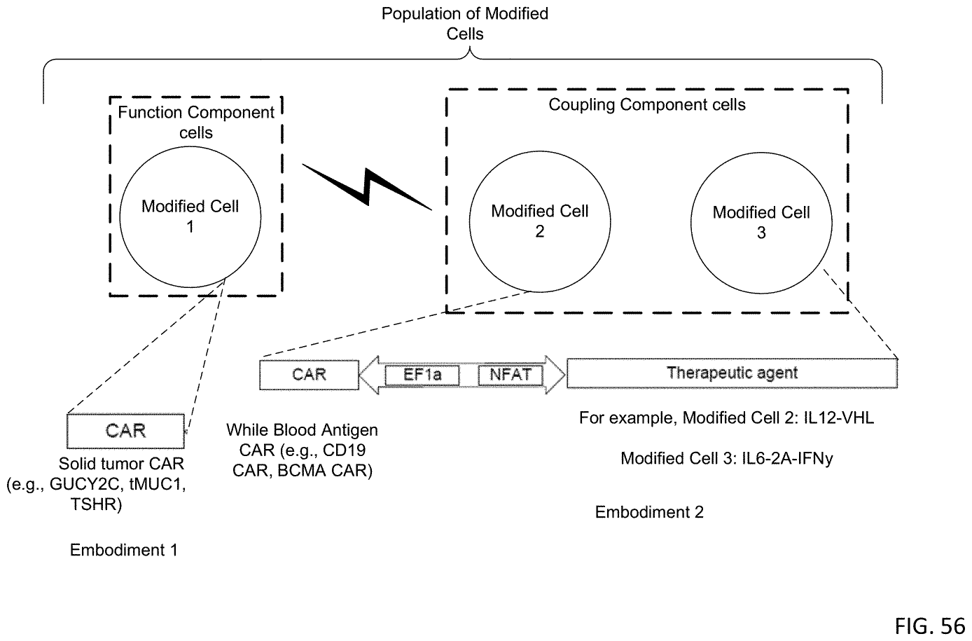

Some embodiments relate to a polynucleotide encoding one or more therapeutic agents comprising at least two cytokines, a modified cell comprising a polynucleotide encoding one or more therapeutic agents (e.g., the modified cell in FIGS. 55 and 56), and/or a method for enhancing T cell response (e.g., expansion and/or activation) in vitro and/or in vivo, the method comprising: introducing a polynucleotide encoding one or more therapeutic agents to obtain a modified cell; culturing the modified cell to obtain a population of modified cells; and contacting the population of modified cells with cells including an antigen, wherein the modified cells enhance the T cell response as compared to the corresponding wild-type cell or a modified cell not comprising the polynucleotide. Some embodiments relate to a population of the modified cells.

In embodiments, the one or more therapeutic agents comprise at least two of IL6, IL12, or IFNγ. In embodiments, the one or more therapeutic agents comprise at least IL12 and IFNγ. In embodiments, the one or more therapeutic agents comprise at least two of IL12, IL6, IFNγ, IFNβ, TNFα, or Neo-2/15. In embodiments, the modified cell comprises a polynucleotide encoding IL12 and IFNγ. In embodiments, the modified cell comprises a polynucleotide encoding TNFα. For example, the polynucleotide encoding TNFα is linked to a HIF VHL binding domain (i.e., VHL domain). For example, a VHL-interaction domain may be linked to a polynucleotide encoding TNF-α. Therefore, when the polynucleotide is introduced into the lymphocytes, TNF-α may be stably expressed in a hypoxic environment (e.g., tumor environments). Thereby, high concentrations of TNF-α in the peripheral blood of the patient in the clinical experiment are avoided. In embodiments, the lymphocytes may further conditionally express a CAR. For example, a VHL-interaction domain of Hif1a may be linked to a polynucleotide encoding a CAR such that the expression of the CAR can be induced by hypoxia.

In embodiments, the population of modified cells comprises two types of cells: function component cells and coupling component cells. The function component cells are capable of inhibiting tumor cells. In embodiments, the function components cells include a binding molecule binding a tumor antigen (e.g., a solid tumor antigen). For example, the binding molecule includes a CAR or a TCR binding solid tumor. In embodiments, the coupling component cells include a CAR targeting a white blood antigen. In embodiments, the coupling component cells include modified cells including a polynucleotide encoding IL-12 linked to a HIF VHL binding domain, and/or modified cells including a polynucleotide encoding IL-6 and IFNγ linked by 2A. An example of the population of modified cells is shown in FIG. 55 or 56.

In embodiments, the one or more therapeutic agents comprise at least one cytokine associated with an oxygen-sensitive polypeptide domain. In embodiments, the oxygen-sensitive polypeptide domain comprises a HIF VHL binding domain. In embodiments, the oxygen-sensitive polypeptide domain is or comprises SEQ ID NO: 457. In embodiments, the polynucleotide encodes a cytokine, an EA linker, and SEQ ID NO: 457. In embodiments, the polynucleotide encodes, or the modified cell comprises at least one of SEQ ID NO: 470-489 and 491-495.

In embodiments, the polynucleotide comprises a promoter comprising a binding site for a transcription modulator that modulates the expression and/or secretion of the therapeutic agent in the cell. In embodiments, the transcription modulator is or includes Hif1a, NFAT, FOXP3, and/or NFkB.

In embodiments, the polynucleotide encodes a binding molecule (e.g., CAR or TCR). In embodiments, the modified cell comprises a binding molecule (e.g., CAR or TCR).

In embodiments, the polynucleotide is present in the modified cell in a recombinant DNA construct, in an mRNA, or in a viral vector. In embodiments, the polynucleotide is an mRNA, which is not integrated into the genome of the modified cell.

In embodiments, the polynucleotide comprises a sequence encoding a cleavable peptide (e.g., 2A or IRES), which is disposed between at least two cytokines.

Some embodiments relate to a composition for treating a subject having cancer, the composition comprising a first population of cells and a second population of cells, wherein the first population of cells comprising a polynucleotide encoding a binding molecule binding a solid tumor antigen, a second population of cells comprising the polynucleotide described herein. In embodiments, the polynucleotide comprises a sequence encoding CAR binding CD19, a sequence encoding IL-6, and a sequence encoding IFNγ. In embodiments, the polynucleotide comprises a sequence encoding CAR binding CD19, a sequence encoding IL-12, and a sequence encoding IFNγ. In embodiments, the binding molecule is a CAR or a TCR. In embodiments, the first population of cells comprises a polynucleotide encoding TNFα that is linked to a HIF VHL binding domain.

Embodiments relate to a composition for treating a subject having cancer, the composition comprising a first population of cells and a second population of cells, wherein the first population of cells comprises a polynucleotide encoding a binding molecule binding a white blood antigen, a second population of cells comprising the polynucleotide described herein. In embodiments, the polynucleotide comprises a sequence encoding a binding molecule binding a solid tumor antigen, a sequence encoding IL-6, and a sequence encoding IFNγ. In embodiments, the polynucleotide comprises a sequence encoding a binding molecule binding a solid tumor antigen, a sequence encoding IL-12, and a sequence encoding IFNγ, wherein the binding molecule is a CAR or a TCR. In embodiments, the first population of cells comprises a polynucleotide encoding TNFα that is linked to a HIF VHL binding domain.

Some embodiments relate to a method of causing or inducing a T cell response, enhancing the T cell response, treating a subject having cancer, or enhancing the treatment, the method comprising: administering an effective amount of the composition described herein to a subject having cancer.

TABLE 2

| | SEQ ID | | SEQ ID | | SEQ ID |
|---|---|---|---|---|---|
| Name | NO: | Name | NO: | Name | No: |
| SP | 1 | UPK2 | 101 | Construct of MUC1- | 201 |
| | | | | 5E5-A-IRES-CD19-A | |
| Hinge & | 2 | ADAM 12 | 102 | CAR 1 of MUC1-5E5- | 202 |
| transmembrane | | | | A-IRES-CD19-A | |
| domain | | | | | |
| Co-stimulatory | 3 | SLC45A3 | 103 | CAR 2 of MUC1-5E5- | 203 |
| domain | | | | A-IRES-CD19-A | |
| CD3-zeta | 4 | ACPP | 104 | Construct of MUC1- | 204 |
| | | | | 5E5-B-IRES-CD19-A | |
| scFV Humanized | 5 | MUC21 | 105 | CAR 1 of MUC1-5E5- | 205 |
| CD19 | | | | B-IRES-CD19-A | |
| scFV CD19 | 6 | MUC16 | 106 | CAR 2 of MUC1-5E5- | 203 |
| | | | | B-IRES-CD19-A | |
| scFv FZD10 | 7 | MS4A12 | 107 | Construct of MUC1- | 206 |
| | | | | 5E5-A-IRES-CD19-B | |
| scFv TSHR | 8 | ALPP | 108 | CAR 1 of MUC1-5E5- | 202 |
| | | | | A-IRES-CD19-B | |
| scFv PRLR | 9 | SLC2A14 | 109 | CAR 2 of MUC1-5E5- | 207 |
| | | | | A-IRES-CD19-B | |
| scFv Muc 17 | 10 | GS1-259H13.2 | 110 | Construct of MUC1- | 208 |
| | | | | 5E5-B-IRES-CD19-B | |
| scFv GUCY2C | 11 | ERVFRD-1 | 111 | CAR 1 of MUC1-5E5- | 205 |
| | | | | B-IRES-CD19-B | |
| scFv CD207 | 12 | ADGRG2 | 112 | CAR 2 of MUC1-5E5- | 207 |
| | | | | B-IRES-CD19-B | |
| Prolactin (ligand) | 13 | ECEL1 | 113 | Construct of MUC1-2- | 209 |
| | | | | A-IRES-CD19-A | |
| scFv CD3 | 14 | CHRNA2 | 114 | CAR 1 of MUC1-2-A- | 210 |
| | | | | IRES-CD19-A | |
| scFv CD4 | 15 | GP2 | 115 | CAR 2 of MUC1-2-A- | 203 |
| | | | | IRES-CD19-A | |
| scFv CD4-2 | 16 | PSG9 | 116 | Construct of MUC1-2- | 211 |
| | | | | B-IRES-CD19-A | |
| scFv CD5 | 17 | SIGLEC15 | 117 | CAR 1 of MUC1-2-B- | 212 |
| | | | | IRES-CD19-A | |
| CD19 antigen | 18 | SLC6A3 | 118 | CAR 2 of MUC1-2-B- | 203 |
| | | | | IRES-CD19-A | |
| FZD10 antigen | 19 | KISS1R | 119 | Construct of MUC1-2- | 213 |
| | | | | A-IRES-CD19-B | |
| TSHR antigen | 20 | QRFPR | 120 | CAR 1 of MUC1-2-A- | 210 |
| | | | | IRES-CD19-B | |
| PRLR antigen | 21 | GPR119 | 121 | CAR 2 of MUC1-2-A- | 207 |
| | | | | IRES-CD19-B | |
| Muc 17 antigen | 22 | CLDN6 | 122 | Construct of MUC1-2- | 214 |
| | | | | B-IRES-CD19-B | |
| GUCY2C antigen | 23 | SP-2 | 123 | CAR 1 of MUC1-2-B- | 212 |
| | | | | IRES-CD19-B | |
| CD207 antigen | 24 | Linker-2 | 124 | CAR 2 of MUC1-2-B- | 207 |
| | | | | IRES-CD19-B | |
| CD3 antigen | 25 | Hinge-2 | 125 | Construct of MUC1- | 215 |
| | | | | 5E5-A-IRES-hCD19-A | |
| CD4 antigen | 26 | TM-2 | 126 | CAR 1 of MUC1-5E5- | 202 |
| | | | | A-IRES-hCD19-A | |
| CD5 antigen | 27 | 4-1BB-2 | 127 | CAR 2 of MUC1-5E5- | 216 |
| | | | | A-IRES-hCD19-A | |
| CAR CD19 nucleic | 28 | CD3 zeta-2 | 128 | Construct of MUC1- | 217 |
| acid | | | | 5E5-B-IRES-hCD19-A | |
| Hinge & TM | 29 | CLDN6-CAR-1 | 129 | CAR 1 of MUC1-5E5- | 205 |
| domain B | | | | B-IRES-hCD19-A | |
| Hinge & TM | 30 | ScFv CLDN6-CAR-1 | 130 | CAR 2 of MUC1-5E5- | 216 |
| domain A | | | | B-IRES-hCD19-A | |
| Hinge & TM | 31 | ScFv VL CLDN6-CAR-1 | 131 | Construct of MUC1- | 218 |
| domain D | | | | 5E5-A-IRES-hCD19-B | |
| Hinge & TM | 32 | ScFv VH CLDN6-CAR-1 | 132 | CAR 1 of MUC1-5E5- | 202 |
| domain C | | | | A-IRES-hCD19-B | |
| Hinge domain D | 33 | CLDN6-CAR-2 | 133 | CAR 2 of MUC1-5E5- | 219 |
| | | | | A-IRES-hCD19-B | |
| Hinge domain C | 34 | ScFv CLDN6-CAR-2 | 134 | Construct of MUC1- | 220 |
| | | | | 5E5-B-IRES-hCD19-B | |
| Hinge domain B | 35 | ScFv VL CLDN6-CAR-2 | 135 | CAR 1 of MUC1-5E5- | 205 |
| | | | | B-IRES-hCD19-B | |
| Hinge domain A | 36 | ScFv VH CLDN6-CAR-2 | 136 | CAR 2 of MUC1-5E5- | 219 |
| | | | | B-IRES-hCD19-B | |
| TM domain D | 37 | CLDN6-CAR-3 | 137 | Construct of MUC1-2- | 221 |
| | | | | A-IRES-hCD19-A | |
| TM domain A | 38 | scFv CLDN6-CAR-3 | 138 | CAR 1 of MUC1-2-A- | 210 |
| | | | | IRES-hCD19-A | |
| CD19 extracellular | 39 | scFv VL CLDN6-CAR-3 | 139 | CAR 2 of MUC1-2-A- | 216 |
| domain | | | | IRES-hCD19-A | |
| TM domain C or B | 40 | scFv VH CLDN6-CAR-3 | 140 | Construct of MUC1-2- | 222 |
| | | | | B-IRES-hCD19-A | |
| WTCD3zeta | 41 | CLDN6-CAR-4 | 141 | CAR 2CAR 1 of | 212 |
| | | | | MUC1-2-B-IRES- | |
| | | | | hCD19-A | |
| WTCD3zeta- | 42 | scFv CLDN6-CAR-4 | 142 | Construct of MUC1-2- | 216 |
| BCMACAR full | | | | B-IRES-hCD19-A | |
| length | | | | | |
| BCMA | 43 | scFv VL CLDN6-CAR-4 | 143 | Construct of MUC1-2- | 223 |
| | | | | A-IRES-hCD19-B | |
| BCMA CAR vector | 44 | scFv VH CLDN6-CAR-4 | 144 | CAR 1 of MUC1-2-A- | 210 |
| | | | | IRES-hCD19-B | |
| BCMA CAR vector | 45 | SIGLEC-15-CAR-1 | 145 | CAR 2 of MUC1-2-A- | 219 |
| | | | | IRES-hCD19-B | |
| VL anti-CD5 | 46 | scFv SIGLEC-15-CAR-1 | 146 | Construct of MUC1-2- | 224 |
| | | | | B-IRES-hCD19-B | |
| VH anti-CD5 | 47 | scFv VL SIGLEC-15- | 147 | CAR 1 of MUC1-2-B- | 212 |
| | | CAR-1 | | IRES-hCD19-B | |
| VL anti-CD4 | 48 | scFv VH SIGLEC-15- | 148 | CAR 2 of MUC1-2-B- | 219 |
| | | CAR-1 | | IRES-hCD19-B | |
| VH anti-CD4 | 49 | VL1 VH1 SIGLEC-15- | 149 | Construct of MUC1- | 225 |
| | | CAR-2 | | 5E5-A-IRES-CD22-A | |
| VL anti-CD3 | 50 | VL1 VH2 SIGLEC-15- | 150 | CAR 1 of MUC1-5E5- | 202 |
| | | CAR-3 | | A-IRES-CD22-A | |
| VH anti-CD3 | 51 | VL1 VH3 SIGLEC-15- | 151 | CAR 2 of MUC1-5E5- | 226 |
| | | CAR-4 | | A-IRES-CD22-A | |
| TSHR extracellular | 52 | VL1 VH 4 SIGLEC-15- | 152 | Construct of MUC1- | 227 |
| domain | | CAR-5 | | 5E5-B-IRES-CD22-A | |
| VH region of | 53 | VL2 VH 1 SIGLEC-15- | 153 | CAR 1 of MUC1-5E5- | 205 |
| BCMA scFv | | CAR-6 | | A-IRES-CD22-A | |
| VL region of BCMA | 54 | VL2 VH2 SIGLEC-15- | 154 | CAR 2 of MUC1-5E5- | 226 |
| scFv | | CAR-7 | | A-IRES-CD22-A | |
| VH region of CD14 | 55 | VL2 VH3 SIGLEC-15- | 155 | Construct of MUC1- | 228 |
| scFv | | CAR-8 | | 5E5-A-IRES-CD22-B | |
| VL region of CD14 | 56 | VL2 VH4 SIGLEC-15- | 156 | MUC1-5E5-A-IRES- | 202 |
| scFv | | CAR-9 | | CD22-B CAR 1 | |
| VH region of CD33 | 57 | VL1 SIGLEC-15-CAR | 157 | MUC1-5E5-A-IRES- | 229 |
| scFv | | | | CD22-B CAR 2 | |
| VL region of CD33 | 58 | VL2 SIGLEC-15-CAR | 158 | MUC1-5E5-B-IRES- | 230 |
| scFv | | | | CD22-B | |
| CD22CAR | 59 | VH1 SIGLEC-15-CAR | 159 | CAR 1 of MUC1-5E5- | 205 |
| | | | | B-IRES-CD22-B | |
| BCMACAR | 60 | VH2 SIGLEC-15-CAR | 160 | CAR 2 of MUC1-5E5- | 229 |
| | | | | B-IRES-CD22-B | |
| MUC1CAR | 61 | VH3 SIGLEC-15-CAR | 161 | Construct of MUC1-2- | 231 |
| | | | | A-IRES-CD22-A | |
| m19CAR-IRES- | 62 | VH4 SIGLEC-15-CAR | 162 | CAR 1 of MUC1-2-A- | 210 |
| MUC1CAR | | | | IRES-CD22-A | |
| hCD19CAR-IRES- | 63 | MUC16-CAR-1 | 163 | CAR 2 of MUC1-2-A- | 226 |
| MUC1CAR | | | | IRES-CD22-A | |
| hCD22CAR-IRES- | 64 | scFv MUC16-CAR-1 | 164 | MUC1-2-B-IRES- | 232 |
| MUC1CAR | | | | CD22-A | |
| BCMACAR-IRES- | 65 | scFv VL MUC16-CAR-1 | 165 | MUC1-2-B-IRES- | 212 |
| MUC1CAR | | | | CD22-A CAR 1 | |
| mCD19CAR-2A- | 66 | scFvVH MUC16-CAR-1 | 166 | MUC1-2-B-IRES- | 226 |
| MUC1CAR | | | | CD22-A CAR 2 | |
| hCD19CAR-2A- | 67 | MUC16-CAR-2 | 167 | MUC1-2-A-IRES- | 233 |
| MUC1CAR | | | | CD22-B | |
| hCD22CAR-2A- | 68 | scFv MUC16-CAR-2 | 168 | MUC1-2-A-IRES- | 210 |
| MUC1CAR | | | | CD22-B CAR 1 | |
| BCMA-2A- | 69 | scFv VL MUC16-CAR-2 | 169 | MUC1-2-A-IRES- | 229 |
| MUC1CAR | | | | CD22-B CAR 2 | |
| Tumor associated | 70 | scFvVH MUC16-CAR-2 | 170 | Construct of MUC1-2- | 234 |
| MUC1 scFv 1 | | | | B-IRES-CD22-B | |
| Tumor associated | 71 | KISS1R-CAR | 171 | CAR 1 of MUC1-2-B- | 212 |
| MUC1 scFv-1 VH | | | | IRES-CD22-B | |
| Tumor associated | 72 | Ligent peptide | 172 | CAR 2 of MUC1-2-B- | 229 |
| MUC1 scFv-1 VL | | KISS1R-CAR | | IRES-CD22-B | |
| Tumor-associated | 73 | ZFLm1 (left) RS aa | 173 | Construct of MUC1- | 235 |
| MUC1 scFv-1 VL | | | | 5E5-A-IRES-CD14-A | |
| CDR 1 | | | | | |
| L2D8-2 (hCAR VL) | 74 | ZFLm1 (left) F1 | 174 | CAR 1 of MUC1-5E5- | 202 |
| | | | | A-IRES-CD14-A | |
| Tumor-associated | 75 | ZFLm1 (left) F2 | 175 | CAR 2 of MUC1-5E5- | 236 |
| MUC1 scFv-1 VL | | | | A-IRES-CD14-A | |
| CDR 3 | | | | | |
| Tumor-associated | 76 | ZFLm1 (left) F3 | 176 | Construct of MUC1- | 237 |
| MUC1 scFv-1 VH | | | | 5E5-B-IRES-CD14-A | |
| CDR 1 | | | | | |
| Tumor associated | 77 | ZFLm1 (left) F4 | 177 | CAR 1 of MUC1-5E5- | 205 |
| MUC1 scFv-1 VH | | | | B-IRES-CD14-A | |
| CDR 2 | | | | | |
| Tumor-associated | 78 | ZFLm1 (left) F5 | 178 | CAR 2 of MUC1-5E5- | 236 |
| MUC1 scFv-1 VH | | | | B-IRES-CD14-A | |
| CDR 3 | | | | | |
| Tumor-associated | 79 | ZFLm1 (left) F6 | 179 | Construct of MUC1- | 238 |
| MUC1 scFv2 | | | | 5E5-A-IRES-CD14-B | |
| Tumor-associated | 80 | ZFRm1-4 (right) RS | 180 | CAR 1 of MUC1-5E5- | 202 |
| MUC1 scFv2 VH | | aa | | A-IRES-CD14-B | |
| Tumor-associated | 81 | ZFRm1-4 (right) F1 | 181 | CAR 2 of MUC1-5E5- | 239 |
| MUC1 scFv2 VL | | | | A-IRES-CD14-B | |
| Tumor-associated | 82 | ZFRm1-4 (right) F2 | 182 | Construct of MUC1-2- | 240 |
| MUC1 scFv-2 VL | | | | A-IRES-CD14-A | |
| CDR 1 | | | | | |
| Tumor-associated | 83 | ZFRm1-4 (right) F3 | 183 | CAR 1 of MUC1-2-A- | 210 |
| MUC1 scFv-2 VL | | | | IRES-CD14-A | |
| CDR 2 | | | | | |
| Tumor-associated | 84 | ZFRm1-4 (right) F4 | 184 | CAR 2 of MUC1-2-A- | 236 |
| MUC1 scFv-2 VL | | | | IRES-CD14-A | |
| CDR 3 | | | | | |
| ′Tumor-associated | 85 | δ chain-1 of Vγ9Vδ2 | 185 | Construct of MUC1-2- | 241 |
| MUC1 scFv-2VH | | | | B-IRES-CD14-A | |
| CDR 1 | | | | | |
| Tumor-associated | 86 | γ chain-2 of Vγ9Vδ2 | 186 | CAR 1 of MUC1-2-B- | 212 |
| MUC1 scFv-2 VH | | | | IRES-CD14-A | |
| CDR 2 | | | | | |
| Tumor-associated | 87 | δ chain-2 of Vγ9Vδ2 | 187 | CAR 2 of MUC1-2-B- | 236 |
| MUC1 scFv-2 VH | | | | IRES-CD14-A | |
| CDR 3 | | | | | |
| GSTA motif | 88 | Vγ9Vδ2 TCR-1: DG. | 188 | Construct of MUC1-2- | 242 |
| | | SF13 | | A-IRES-CD14-B | |
| | | γ chain | | | |
| Modified PD-1 | 89 | Vγ9Vδ2 TCR-1: DG. | 189 | CAR 1 of MUC1-2-A- | 210 |
| intracellular | | SF13 δ chain | | IRES-CD14-B | |
| domain -1 | | | | | |
| Modified PD-1 | 90 | Vγ9Vδ2 TCR-2: DG. | 190 | CAR 2 of MUC1-2-A- | 239 |
| intracellular | | SF68: | | IRES-CD14-B | |
| domain -2 | | γ chain | | | |
| Modified PD-1 | 91 | Vγ9Vδ2 TCR-2: DG. | 191 | Construct of MUC1-2- | 243 |
| intracellular | | SF68: | | B-IRES-CD14-B | |
| domain -3 | | δ chain | | | |
| Modified PD-1 | 92 | Vγ9Vδ2 TCR-3: | 192 | CAR 1 of MUC1-2-B- | 212 |
| intracellular | | 12G12: | | IRES-CD14-B | |
| domain -4 | | γ chain | | | |
| Modified PD-1 | 93 | Vγ9Vδ2 TCR-3: | 193 | CAR 2 of MUC1-2-B- | 239 |
| intracellular | | 12G12: | | IRES-CD14-B | |
| domain -5 | | δ chain | | | |
| Removed PD-1 | 94 | Vγ9Vδ2 TCR-4: | 194 | Construct of MUC1- | 244 |
| intracellular | | CP.1.15 | | 5E5-A-IRES-BCMA-A | |
| domain -1 | | γ chain | | | |
| Removed PD-1 | 95 | TCR-4: CP. 1.15δ | 195 | CAR 1 of MUC1-5E5- | 202 |
| intracellular | | chain | | A-IRES-BCMA-A | |
| domain -2 | | | | | |
| FokI WC | 96 | WT CD3-zeta | 196 | CAR 2 of MUC1-5E5- | 245 |
| | | | | A-IRES-BCMA-A | |
| M FokI | 97 | Invariant sequence for | 197 | Construct of MUC1- | 246 |
| | | iNKT a chain (hVα24- | | 5E5-B-IRES-BCMA-A | |
| | | JαQ-TRAC) | | | |
| M FokI | 98 | An example of iNKT β | 198 | CAR 1 of MUC1-5E5- | 205 |
| | | chain sequence | | B-IRES-BCMA-A | |
| | | (containing Vβ11): | | | |
| γ chain-1 of | 99 | Invariant sequence for | 199 | CAR 2 of MUC1-5E5- | 245 |
| Vγ9Vδ2 | | MAIT α chain | | B-IRES-BCMA-A | |
| | | (hAV7S2-AJ33 α | | | |
| | | chain) (version1) | | | |
| VL anti-CD4-2 | 100 | VH anti- CD4-2 | 200 | Construct of MUC1- | 247 |
| | | | | 5E5-A-IRES-BCMA-B | |
| CAR 1 of MUC1-2- | 210 | CAR 1 of MUC1-5E5- | 205 | CAR 1 of MUC1-5E5- | 202 |
| A-IRES-CD33-A | | B-IRES-CD33-A | | A-IRES-BCMA-B | |
| CAR 2 of MUC1-2- | 255 | CAR 2 of MUC1-5E5- | 255 | CAR 2 of MUC1-5E5- | 248 |
| A-IRES-CD33-A | | B-IRES-CD33-A | | A-IRES-BCMA-B | |
| Construct ofMUCI- | 261 | Construct ofMUC1- | 257 | Construct of MUC1- | 249 |
| 2-B-IRES-CD33-A | | 5E5-A-IRES-CD33-B | | 5E5-B-IRES-BCMA-B | |
| CAR 1 of MUC1-2- | 212 | CAR 1 of MUC1-5E5- | 202 | CAR 1 of MUC1-5E5- | |
| B-IRES-CD33-A | | A-IRES-CD33-B | | B-IRES-BCMA-B | |
| CAR 2 of MUC1-2- | 255 | CAR 2 of MUC1-5E5- | 258 | CAR 2 of MUC1-5E5- | |
| B-IRES-CD33-A | | A-IRES-CD33-B | | B-IRES-BCMA-B | |
| Construct ofMUC1- | 262 | Construct ofMUC1- | 259 | Construct of MUC1-2- | 250 |
| 2-A-IRES-CD33-B | | 5E5-B-IRES-CD33-B | | A-IRES-BCMA-A | |
| CAR 1 of MUC1-2- | 210 | CAR 1 of MUC1-5E5- | 205 | CAR 1 of MUC1-2-A- | 210 |
| A-IRES-CD33-B | | B-IRES-CD33-B | | IRES-BCMA-A | |
| CAR 2 of MUC1-2- | 258 | CAR 2 of MUC1-5E5- | 258 | CAR 2 of MUC1-2-A- | 245 |
| A-IRES-CD33-B | | B-IRES-CD33-B | | IRES-BCMA-A | |
| Construct ofMUC1- | 263 | Construct ofMUC1-2- | 260 | Construct of MUC1-2- | 251 |
| 2-B-IRES-CD33-B | | A-IRES-CD33-A | | B-IRES-BCMA-A | |
| CAR 1 of MUC1-2- | 212 | Construct ofMUC1-2- | 253 | CAR 1 of MUC1-2-B- | 212 |
| B-IRES-CD33-B | | B-IRES-BCMA-B | | IRES-BCMA-A | |
| CAR 2 of MUC1-2- | 258 | CAR 1 of MUC1-2-B- | 212 | CAR 2 of MUC1-2-B- | 245 |
| B-IRES-CD33-B | | IRES-BCMA-B | | IRES-BCMA-A | |
| Construct ofMUC1- | 254 | MUC1-2-B-IRES- | 248 | Construct of MUC1-2- | 252 |
| 5E5-A-IRES- | | BCMA-B CAR 2 | | A-IRES-BCMA-B | |
| CD33-A | | | | | |
| CAR 1 of MUC1- | 202 | MUC1-5E5-B-IRES- | 256 | CAR 1 of MUC1-2-A- | 210 |
| 5E5-A-IRES- | | CD33-A | | IRES-BCMA-B | |
| CD33-A | | | | | |
| CAR 2 of MUC1- | 255 | CAR 2 of MUC1-2-A- | 248 | MUC1-5e5Panko- | 264 |
| 5E5-A-IRES- | | IRES-BCMA-B | | enhanced scFc | |
| CD33-A | | | | | |
| MUC1-Panko5e5 - | 265 | hinge and/or | 266 | hinge and/or | 267 |
| enhanced scFc | | transmembrane | | transmembrane | |
| | | domain A | | domain B | |
| hinge and/or | 268 | hinge and/or | 269 | MUC1-5e5Panko- | 270 |
| transmembrane | | transmembrane | | enhanced scFc | |
| domain C | | domain D | | A 41BB CD2 zeta | |
| MUC1-5e5Panko- | 271 | MUC1-5e5Panko- | 272 | MUC1-5e5Panko- | 273 |
| enhanced | | enhanced scFc | | enhanced scFc | |
| scFc B 41BB CD2 | | C 41BB CD2 zeta | | D 41BB CD2 zeta | |
| zeta | | | | | |
| MUC1-Panko5e5- | 274 | MUC1-Panko5e5- | 275 | MUC1-Panko5e5- | 276 |
| enhanced | | enhanced scFc | | enhanced scFc C | |
| scFc A 41BB CD2 | | B 41BB CD2 zeta | | 41BB CD2 zeta | |
| zeta | | | | | |
| MUC1-Panko5e5- | 277 | GS linker | 278 | Construct of TSHR | 279 |
| enhanced | | | | CAR | |
| scFc D 41BB CD2 | | | | | |
| zeta | | | | | |
| CD8a Hinge & | 280 | IL-17C | 296 | IL12- IgG4 Hinge | 313 |
| transmembrane | | Nucleic acid Sequence | | &CD8a | |
| | | | | transmembrane | |
| CD8a | 281 | IL-17C | 297 | IL12Rβ2 cytoplasmic | 314 |
| transmembrane | | aa Sequence | | | |
| IgG4 Hinge&CD8a | 282 | IL-17D | 298 | IL18R1 cytoplasmic | 315 |
| transmembrane | | Nucleic acid Sequence | | | |
| IL-2 | 283 | IL-17D | 299 | IL23R cytoplasmic | 316 |
| Nucleic acid | | aa Sequence | | | |
| Sequence | | | | | |
| IL-2 | 285 | IL-17F | 300 | Gp130 (IL6ST) | 317 |
| aa Sequence | | Nucleic acid Sequence | | cytoplasmic | |
| IL-6 | 286 | IL-17F | 301 | IL15Ra, cytoplasmic | 318 |
| Nucleic acid | | aa Sequence | | | |
| Sequence | | | | | |
| IL-6 | 287 | IL-23A | 302 | IL12Rβ1 | 319 |
| aa Sequence | | Nucleic acid Sequence | | cytoplasmic | |
| IL-7 | 288 | IL-23A | 303 | 41BB + cd3zeta + IL | 320 |
| Nucleic acid | | aa Sequence | | receptor cytoplasmic | |
| Sequence | | | | region | |
| IL-7 | 289 | IL-18 | 304 | scFv + hinge + | 321 |
| aa Sequence | | Nucleic acid Sequence | | transmembrane + 41BB + | |
| | | | | IL receptor cytoplasmic | |
| | | | | region + cd3zeta | |
| IL-15 | 290 | IL-18 | 305 | scFv + hinge + | 322 |
| Nucleic acid | | aa Sequence | | transmem brane + IL | |
| Sequence | | | | receptor cytoplasmic | |
| | | | | region + 41BB + | |
| | | | | cd3zeta | |
| IL-15 | 291 | IL-12 αp35 | 306 | 41BB promoter | 323 |
| aa Sequence | | Nucleic acid Sequence | | | |
| IL-17A | 292 | IL-12αp35 | 307 | CD25 enhancer + | 324 |
| Nucleic acid | | aa Sequence | | minimal TK promoter | |
| Sequence | | | | | |
| IL-17A | 293 | IL-12βp40 | 308 | CD69 enhancer + | 325 |
| aa Sequence | | Nucleic acid Sequence | | minimal TK promoter | |
| IL-17B | 294 | IL-12 βp40 | 309 | IFN-gamma promoter | 326 |
| Nucleic acid | | aa Sequence | | | |
| Sequence | | | | | |
| IL-17B | 295 | Fusion IL-12 | 310 | 2A | 327 |
| aa Sequence | | | | | |
| Hypoxia promoter | 330 | Fusion IL-23 | 311 | FN-γ | 328 |
| (example 2) | | | | | |
| Transcription factor | | | | | |
| binding sites. | | | | | |
| Hypoxia promoter | 331 | IL-12- CD8a Hinge & | 312 | hPDE5 | 329 |
| (example 3) | | transmembrane | | | |
| Transcription factor | | | | | |
| binding sites. | | | | | |
| NFAT promoter | 332 | Hypoxia promoter | 338 | TNF-alpha | 347 |
| (example 1) | | (example 1) the | | | |
| Transcription factor | | minimal promoter | | | |
| binding sites. | | | | | |
| NFAT promoter | 333 | Hypoxia promoter | 339 | Lymphotoxin | 348 |
| (example 2) | | (example 2) the | | beta (TNF-C) | |
| Transcription factor | | minimal promoter | | | |
| binding sites. | | | | | |
| FOXP3 promoter | 334 | Hypoxia promoter | 340 | OX40L | 349 |
| (example1) | | (example 3) the | | | |
| Transcription factor | | minimal promoter | | | |
| binding sites. | | | | | |
| Hypoxia promoter | 335 | NFAT promoter | 341 | CD154 | 350 |
| (example 1) | | (example 1) the | | | |
| Transcription factor | | minimal promoter | | | |
| binding sites. | | | | | |
| NFkB promoter | 336 | NFAT promoter | 342 | FasL | 351 |
| (example1) | | (example 2) the | | | |
| Transcription factor | | minimal promoter | | | |
| binding sites. | | | | | |
| NFkB promoter | 337 | FOXP3 promoter | 343 | CD70 | 352 |
| (example2) | | (example1) the | | | |
| Transcription factor | | minimal promoter | | | |
| binding sites. | | | | | |
| CXCI1 | 364 | FOXP3 promoter | 344 | CD153 | 353 |
| | | (example2) the | | | |
| | | minimal promoter | | | |
| CXCL2 | 365 | NFkB promoter | 345 | 4-1BB ligand | 354 |
| | | (example1) the | | | |
| | | minimal promoter | | | |
| CXCL3 | 366 | NFkB promoter | 346 | TRAIL | 355 |
| | | (example2) the | | | |
| | | minimal promoter | | | |
| CXCL4 | 367 | CXCL11 | 374 | RANKL | 356 |
| CXCL5 | 368 | CXCL12 | 375 | TWEAK | 357 |
| CXCL6 | 369 | CXCL13 | 376 | APRIL | 358 |
| CXCL7 | 370 | CXCL14 | 377 | LIGHT | 359 |
| CXCL8 | 371 | CCL1 | 378 | NGF, | 360 |
| CXCL9 | 372 | CCL2 | 379 | TNFSF18 | 361 |
| CXCL10 | 373 | CCL3 | 380 | TNFSF15 | 362 |
| ICOS ligand | 405 | CCI3L1 | 381 | Ectodysplasin-A | 363 |
| CD80 | 406 | CCI4 | 382 | IL2 receptor CD | 415 |
| CD86 | 407 | CCI5 | 383 | IL6 receptor CD | 418 |
| scFv against PD1 | 408 | CCI7 | 384 | IL7 receptor CD | 421 |
| scFv against PDL1 | 409 | CCI8 | 385 | IL12 receptor CD | 424 |
| B7-H3 scFv 1 | 410 | CCI11 | 386 | IL15 receptor CD | 427 |
| B7-H3 scFv2 | 411 | CCL13 | 387 | IL21 receptor CD | 430 |
| B7-H3 scFv3 | 412 | CCI14 | 388 | IL23 receptor CD | 433 |
| IL2 receptor ED | 413 | CCL15 | 389 | CD4 TM | 434 |
| IL6 receptor ED | 416 | CCI16 | 390 | CD8 TM | 436 |
| IL7 receptor ED | 419 | CCL17 | 391 | CD27 TM | 438 |
| IL12 receptor ED | 422 | CCI18 | 392 | CD28 TM | 440 |
| IL15 receptor ED | 425 | CCL19 | 393 | CD137 TM | 442 |
| IL21 receptor ED | 428 | CCL20 | 394 | PD1 TM | 444 |
| IL23 receptor ED | 431 | CCL21 | 395 | PDL1 TM | 446 |
| IL2 receptor TM | 414 | CCL22 | 396 | CD4 CD | 435 |
| IL6 receptor TM | 417 | CCL23 | 397 | CD8 CD | 437 |
| IL7 receptor TM | 420 | CCL24 | 398 | CD27 CD | 439 |
| IL12 receptor TM | 423 | CCL25 | 399 | CD28 CD | 441 |
| IL15 receptor TM | 426 | CCL26 | 400 | CD137 CD | 443 |
| IL21 receptor TM | 429 | CCL27 | 401 | PD1 CD | 445 |
| IL23 receptor TM | 432 | CCL28 | 402 | PDL1 CD | 447 |
| IL2 | 448 | Lymphotactin | 403 | IL21 | 452 |
| IL7 | 449 | CX3CL1 | 404 | IL23 | 453 |
| IL12 | 450 | Hif VHL-interaction | 457 | IL33 | 454 |
| | | domain:Hif amino | | | |
| | | acid 344-417 | | | |
| IL15 | 451 | Hif amino acid 380-603 | 458 | TNFα | 455 |
| siglec-15 antigen 2 | 460 | siglec-15 antigen 1 | 459 | IFNγ point mutation | 456 |
| siglec-15 antigen 3 | 461 | siglec-15 antigen 6 | 464 | GS linker sequence | 467 |
| siglec-15 antigen 4 | 462 | siglec-15 antigen 7 | 465 | EA linker sequence | 468 |
| siglec-15 antigen 5 | 463 | siglec-15 antigen 8 | 466 | NFAT6x + minimal | 469 |
| | | | | IL12 promoter | |
| IL12&IFN-γ | 470 | IFN-γ&IL12 | 471 | IL12-VHL 1 | 472 |
| IL12-VHL 2 | 473 | IFN-γ-VHL1 | 474 | IFN-γ-VHL2 | 475 |
| IL6-VHL1 | 476 | IL6-VHL2 | 477 | IL6& FN-γ | 478 |
| IFN-γ&IL6 | 479 | IL12&IFN-γm | 480 | IFN-γm&IL12 | 481 |
| IFN-γm-VHL1 | 482 | IFN-γm-VHL2 | 483 | TNFα VHL1 | 484 |
| TNFα VHL 2 | 485 | IL12 & IL6 | 486 | IL6 & IL12 | 487 |
| IL6& IFN-γm | 488 | IFNm-γ&IL6 | 489 | Neo-2/15 | 490 |
| Neo-2/15-VHL 1 | 491 | Neo-2/15-VHL 2 | 492 | IFNβ-VHL 1 | 493 |
| IFNβ-VHL 2 | 494 | T2A (GSG residues | 495 | P2A(GSG residues are | 496 |
| | | are optional) | | optional) | |
| E2A(GSG residues | 497 | F2A(GSG residues are | 498 | TCF-LEF response | 510 |
| are optional) | | optional) | | element | |
| Stat5 response | 499 | stat5 response | 500 | Stat3 response | 501 |
| element | | element + minimal | | element | |
| | | promoter | | | |
| stat3 response | 502 | Interferon Stimulated | 503 | Interferon Stimulated | 504 |
| element + minimal | | Response Element | | Response Element + | |
| promoter | | | | minimal promoter | |
| AP1 Response | 505 | SMAD Binding | 506 | Serum Response | 507 |
| Element | | Element | | Element | |
| Serum Response | 508 | Cyclic AMP response | 509 | IFNA2 | 511 |
| Factor Response | | element | | | |
| Element | | | | | |
| IFNA4 | 512 | IFNA14 | 518 | IFNA1 | 524 |
| IFNA5 | 513 | IFNA16 | 519 | IFNA21 | 525 |
| IFNA6 | 514 | IFNA17 | 520 | IRF8 | 526 |
| IFNA7 | 515 | IFNB1 | 521 | IRF4 | 527 |
| IFNA8 | 516 | IFNK | 522 | NOTCH2 | 528 |
| IFNA10 | 517 | IFNW1 | 523 | KLF4 | 529 |
| BATF3 | 530 | TCF4 (E2-2) | 531 | hCD19-CAR (4-1BB + | 532 |
| | | | | CD3 zeta) -NATF-IL6- | |
| | | | | 2A-IFNγ | |
| hCD19-CAR(4- | 533 | scFv ACPP | 534 | GUCY2C-CAR | 535 |
| 1BB + CD3 zeta)- | | | | | |
| NATF-IL12-VHL | | | | | |
| Vector | 284 | | | GS linker | 536 |
| including CAR | | | | | |
| and IL12 in series | | | | | |

TM: Transmembrane domain
CD: cytoplasmic domain EM: Extracellular daemon

TABLE 3

| transcription | Expression Conditions | |
|---|---|---|
| factors | or notes | Example of constructs |
| Hif1a | hypoxia-induced | Hif1a binding site + minimal |
| | expression | promoter + CDS of IL |
| NFAT | transcription factor | NFAT binding site + minimal |
| | in an immune response | promoter + CDS of IL |
| FOXP3 | transcription factor | FOXP3 binding site + minimal |
| | in T-reg | promoter + CDS of IL |
| NFkB | transcription factor | NFkB binding site + minimal |
| | in an immune response | promoter + CDS of IL |

TABLE 4

| isolated | |
|---|---|
| nucleic acid | |
| sequence | Nucleic Acid Construct using Encoded Peptides |
| 1 | CAR + P2A + IL + CD8a Hinge & transmembrane |
| 2 | CAR + P2A + IL + IgG4 Hinge & CH2CH3 & CD4 |
| | transmembrane |
| 3 | CAR + Hypoixa/NFAT/FOXP3/NFkB promoter + IL + |
| | CD8a Hinge & transmembrane |
| 4 | CAR + Hypoixa/NFAT/FOXP3/NFkB promoter + IL + |
| | CD8a IgG4Hinge & CH2CH3 & CD4 transmembrane |
| 5 | scFv + hinge + transmembrane + 41BB + cd3zeta + |
| | IL receptor cytoplasmic region |
| 6 | scFv + hinge + transmembrane + 41BB + IL receptor |
| | cytoplasmic region + cd3zeta |
| 7 | scFv + hinge + transmembrane + IL receptor cytoplasmic |
| | region + 41BB + cd3zeta |

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### Exemplary Embodiments

The following are exemplary embodiments:

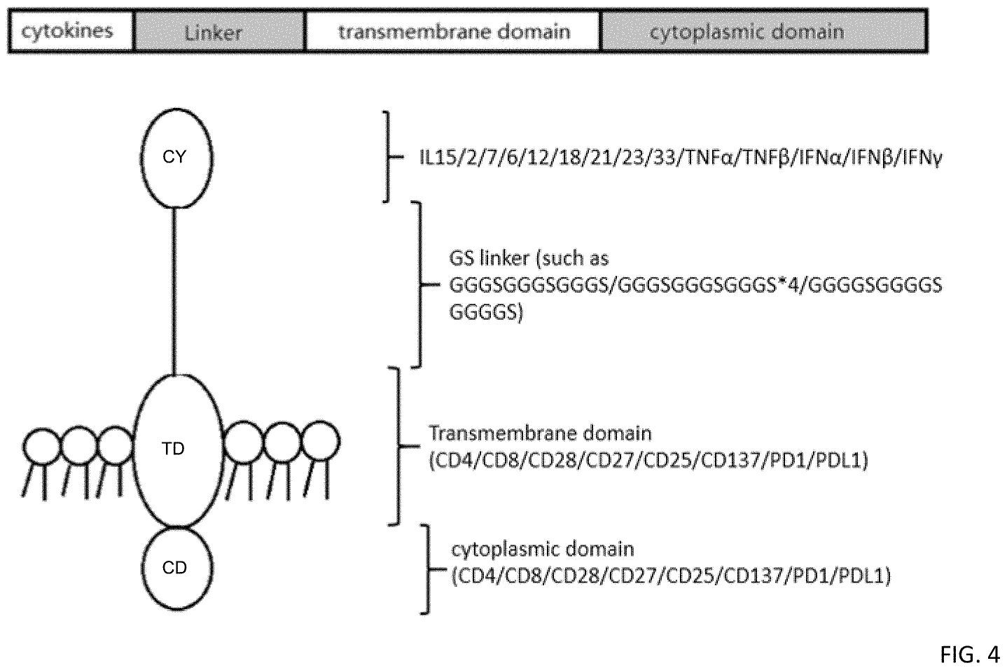

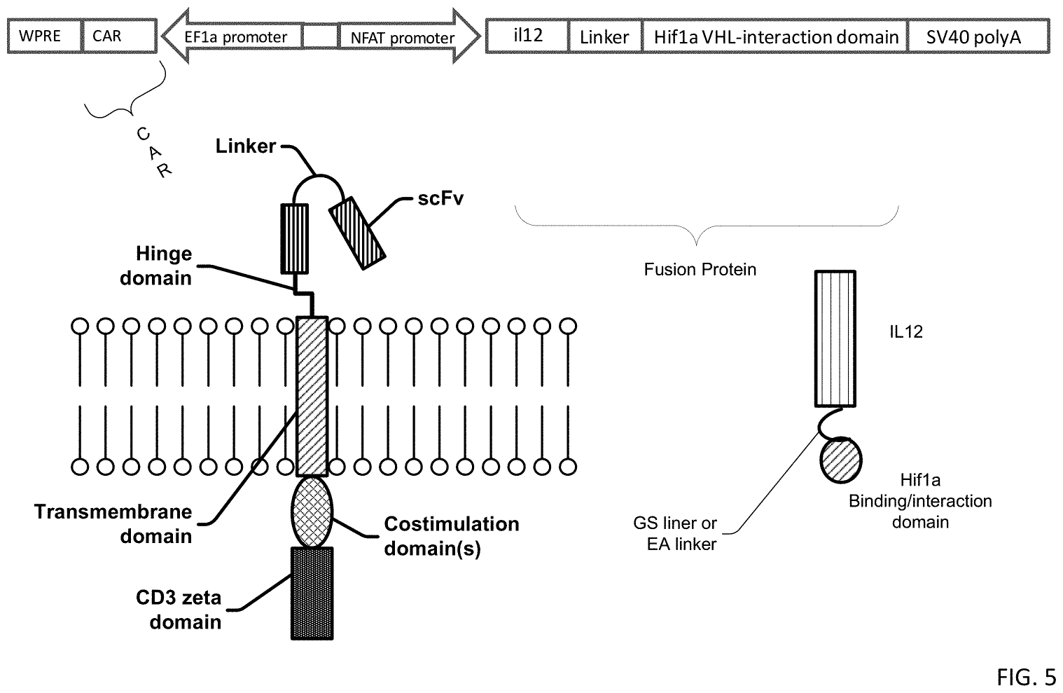

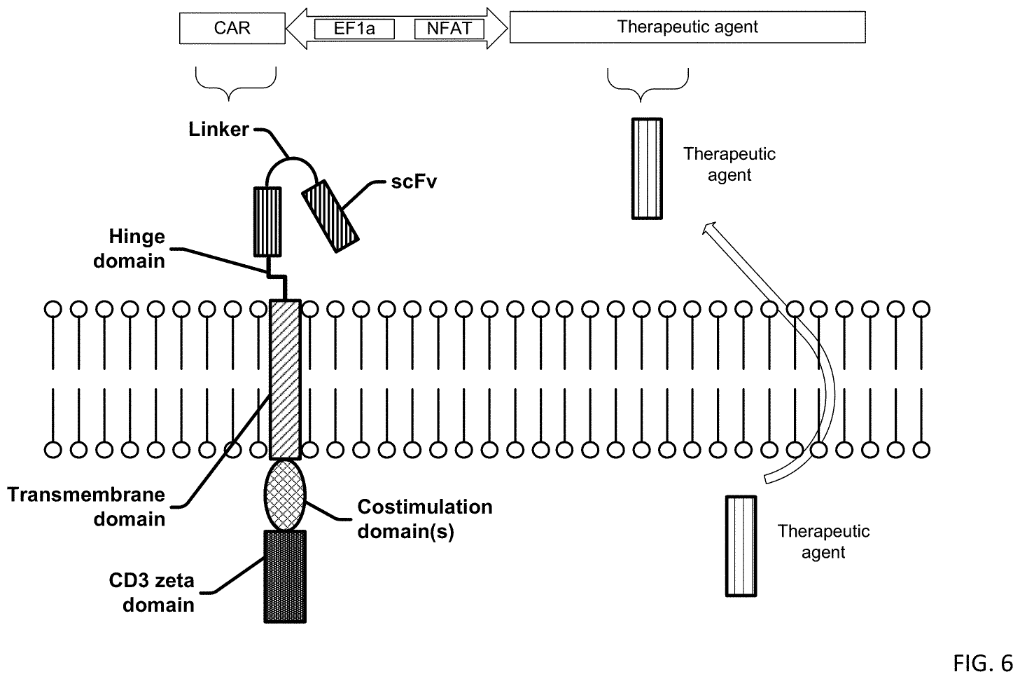

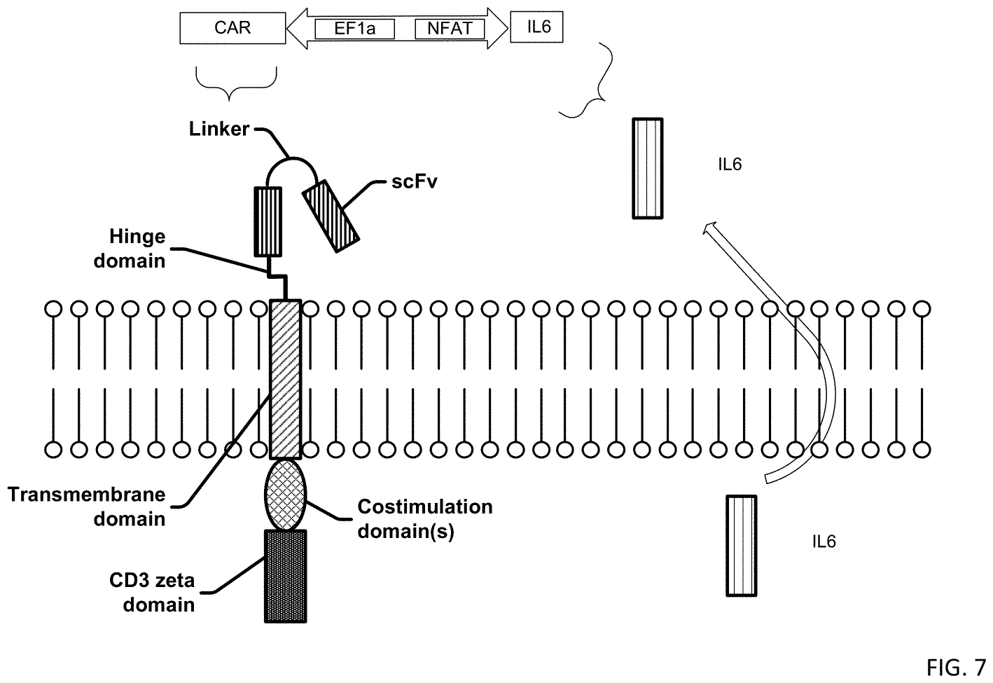

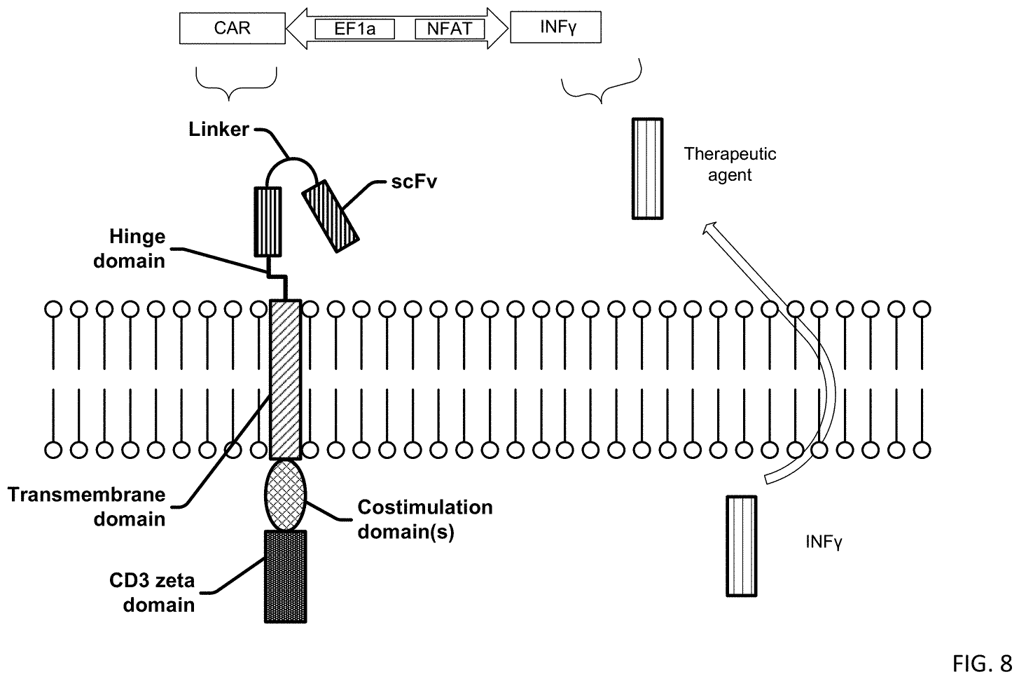

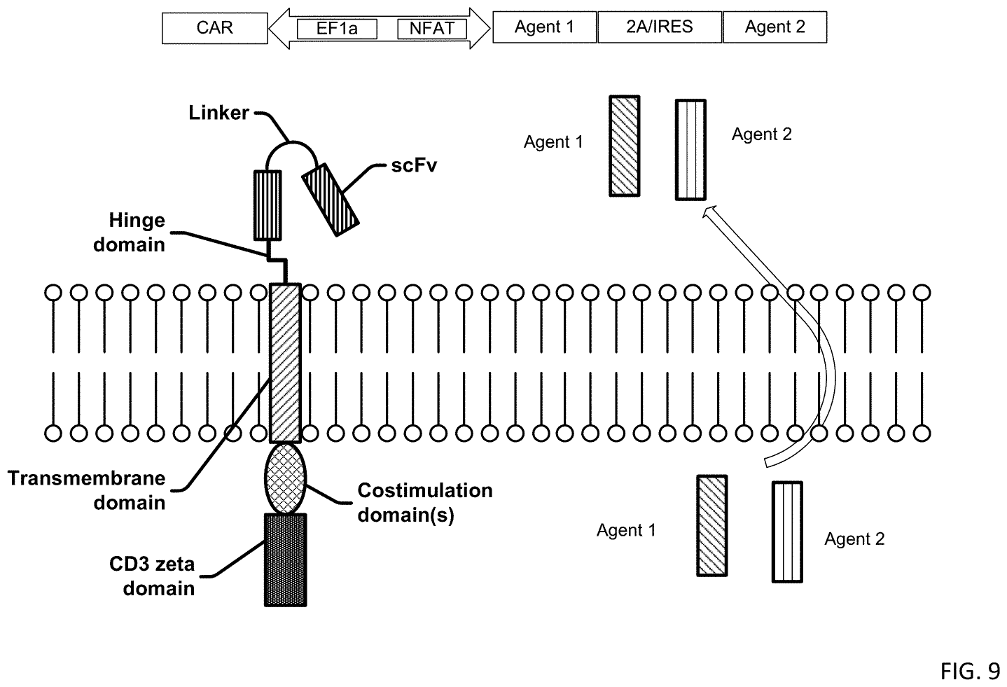

1. An isolated polynucleotide comprising a first polynucleotide and a second or an additional polynucleotide, the first polynucleotide encoding a chimeric antigen receptor (CAR), the second or additional polynucleotide encoding one or more therapeutic agents, the one or more therapeutic agents are or comprise of IFNγ, IL-2, IL-6, IL-7, IL-12, IL-15, IL-17, IL-18, IL-23, or a combination thereof.
2. An isolated polynucleotide comprising a first polynucleotide and a second or an additional polynucleotide, the first polynucleotide encoding a chimeric antigen receptor (CAR), the second or additional polynucleotide encoding a therapeutic agent that is or comprises at least one of TNFRSF superfamily member receptor activation antibodies or membrane-bound forms thereof, TNFRSF superfamily member ligands or the membrane-bound form thereof, chemokines or membrane-bound forms thereof, antibodies to the chemokines, antibodies to receptors of the chemokines or the membrane-bound forms thereof, or D28 family's ligands that correspond to the sequences in Table 2-4.
3. A population of CAR cells comprising the first polynucleotide and the second or additional polynucleotide of embodiments 1 or 2, wherein the CAR cells comprise lymphocyte, leukocyte, or PBMC.
4. The population of CAR cells of embodiment 3, wherein the CAR and the one or more therapeutic agents are produced in the form of a polyprotein, which is cleaved to generate separate CAR and therapeutic agent molecules.
5. The population of CAR cells of embodiment 4, wherein the polyprotein comprises a cleavable moiety between the CAR and the therapeutic agent, the cleavable moiety comprises a 2A peptide, the 2A peptide comprises P2A or T2A, and/or the CAR and the therapeutic agent are each constitutively expressed.
6. The population of CAR cells of embodiment 3, wherein the CAR cells comprise: a third polynucleotide encoding a second or an additional CAR binding an antigen that is different from the antigen that the first CAR binds, the second or additional CAR binding a solid tumor antigen, and the first CAR binding an antigen of a white blood cell.
7. A pharmaceutical composition comprising the population of CAR cells of any one of embodiments 3-6, wherein the pharmaceutical composition is used to treat a patient having a solid tumor and/or lymphoma. For example, the CAR cells are CD19CAR T cells expressing IL-6, IL-12, and/or IFNγ may be used to treat lymphoma.
8. A method of inducing or causing a T cell response in a subject in need thereof and/or treating a tumor of the subject, the method comprising administering an effective amount of the composition of embodiment 7 to the subject.
9. A modified cell comprising one or more CARs, wherein the cell is engineered to express and secrete a therapeutic agent that is or comprises at least one of IFNγ, IL-2, IL-6, IL-7, IL-12, IL-15, IL-17, IL-18, or IL-23.
10. A method of inducing or causing or enhancing T cell response, treating cancer, or enhancing cancer treatment, the method comprising: administrating to a subject in need thereof an effective amount of a composition of T cells comprising one or more CARs, wherein the cell is engineered to express and secrete one or more therapeutic agents, wherein the therapeutic agent is or comprises IFNγ, IL-2, IL-6, IL-7, IL-12, IL-15, IL-17, IL-18, IL-23, or a combination thereof and the T cell response in the subject is enhanced as compared to the T cell response in the subject administered with T cells that do not express or secrete the therapeutic agent.
11. A method of inducing or causing or enhancing T cell response, treating cancer, or enhancing cancer treatment, the method comprising: administering to a subject in need thereof an effective amount of the composition of a population of T cells comprising a CAR; and administering to the subject an effective amount of one or more therapeutic agents, wherein the therapeutic agent is or comprises IFNγ, IL-2, IL-6, IL-7, IL-12, IL-15, IL-17, IL-18, IL-23, or a combination thereof, and wherein the T cell response in the subject is enhanced as compared to the T cell response in the subject administered with a population of T cells comprising a CAR without being administered a therapeutic agent.
12. The method of embodiment 11, wherein administering an effective amount of the therapeutic agent comprises intravenous delivery of an amount of human IL-6 in the range of about 0.5-50 ug per kilogram of body weight.
13. The modified cell or the method of any one of embodiments 9-12, wherein the T cell comprises a second or an additional CAR binding a solid tumor antigen, and the first CAR binds an antigen of a white blood cell.
14. The modified cell or the method of embodiment 13, wherein the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the antigen of a white blood cell is a B cell antigen. For example, the B cell antigen is CD19, CD20, CD22, or BCMA.
15. The isolated polynucleotide, the modified cell, or the method of any one of embodiments 2-14, wherein the therapeutic agent is IL-6 or IL-7.
16. The isolated polynucleotide, the modified cell, or the method of any one of embodiments 2-14, wherein the CAR comprises an extracellular domain, a transmembrane domain, and an intracellular domain, the extracellular domain binding an antigen.
17. The isolated polynucleotide, the modified cell, or the method of embodiment 16, wherein the intracellular domain comprises a co-stimulatory domain that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.
18. The isolated polynucleotide, the modified cell, or the method of embodiment 17, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
19. The isolated polynucleotide, the modified cell, or the method of any one of embodiments 2-18, wherein the modified cell or T cells comprise a dominant negative form of PD-1 mutant such that PD-1/PDL-1 signaling pathway of the cell is interfered with or blocked.
20. The isolated polynucleotide, the modified cell, or the method of any one of embodiments 2-19, wherein the polynucleotide encoding the therapeutic agent is present in the modified cell in a recombinant DNA construct, in an mRNA, or in a viral vector.
21. The isolated polynucleotide, the modified cell, or the method of any one of embodiments 2-19, wherein the modified cell comprises an mRNA encoding the therapeutic agent, wherein the mRNA is not integrated into the genome of the modified cell.
22. The isolated polynucleotide, the modified cell, or the method of any one of embodiments 2-21, wherein the therapeutic agent corresponds to at least one of sequence listed in Table 2-4.
23. The isolated polynucleotide, the modified cell, or the method of any one of embodiments 2-21, wherein the modified cell comprises a polynucleotide comprising a promoter comprising a binding site for a transcription modulator that modulates the expression of the therapeutic agent in the cell.
24. The isolated polynucleotide, the modified cell, or the method of embodiment 23, wherein the transcription modulator is or includes Hif1a, NFAT, FOXP3, and/or NFkB.
25. The isolated polynucleotide, the modified cell, or the method of embodiment 24, wherein the promoter is responsive to the transcription modulator.
26. The isolated polynucleotide, the modified cell, or the method of embodiment 25, wherein the promoter is operably linked to the polynucleotide encoding the therapeutic agent such that the promoter drives expression of the therapeutic agent in the cell.
27. The isolated polynucleotide, the modified cell, or the method of embodiment 23, wherein the promoter and the binding site correspond to the sequences listed in Table 2-4.
28. An isolated polynucleotide comprising a first polynucleotide and a second polynucleotide, the first polynucleotide encoding a chimeric antigen receptor (CAR), the second polynucleotide encoding a therapeutic agent and a transmembrane domain such that the therapeutic agent is associated or bound to cell membrane. Examples of the isolated polynucleotide are listed in Table 4 (1-4)
29. A modified cell comprising the isolated polynucleotide of embodiment 28.
30. A pharmaceutical composition comprising the population of the cells of embodiment 3.
31. A method of inducing or causing T cell response in a subject in need thereof and/or treating a tumor of the subject (e.g., solid tumor and/or lymphoma (e.g., CD19 CAR), the method comprising administering an effective amount of the composition of embodiment 30 to the subject.
32. A modified cell comprising a first polynucleotide encoding a CAR, and a second polynucleotide encoding a therapeutic agent and a transmembrane domain such that the therapeutic agent is associated or bound to the membrane of the modified cell.
33. The modified cell of any one of embodiments 29-32, wherein the therapeutic agent is a cytokine.
34. The modified cell of embodiment 33, wherein the cytokine comprises multiple subunits, the second nucleic acid encodes the multiple subunits, one or more linkers connecting the multiple subunits, and the transmembrane domain.
35. The modified cell of embodiment 33, wherein the second polynucleotide comprises a polynucleotide of SEQ ID NO: 283, 284, 286, 288, 290, 292, 294, 296, 298, 300, 302, 304, 306, or 308, or a polynucleotide that encodes an amino acid sequence of SEQ ID NO: 281, 282, 285, 287, 289, 291, 293, 295, 297, 299, 301, 303, 305, 307, 309, 310, 311, 312, or 313.
36. The modified cell of embodiment 33, wherein the cytokine is or comprises at least one of IFNγ, IL-2, IL-6, IL-7, IL-12, IL-15, IL-17, IL-18, or IL-23.
37. The modified cell of any one of embodiments 29-36, wherein the CAR comprises an extracellular domain, a transmembrane domain, and an intracellular domain, wherein the extracellular domain binds an antigen.
38. The modified cell of embodiment 37, wherein the intracellular domain comprises a co-stimulatory domain that comprises an intracellular domain of a co-stimulatory molecule selected from the group consisting of CD27, CD28, 4-11BB, OX40, CD30, CD40, PD-1, ICOS, lymphocyte function-associated antigen-1 (LFA-1), CD2, CD7, LIGHT, NKG2C, B7-H3, and any combination thereof.
39. The modified cell of embodiment 37, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
40. The modified cell of any one of embodiments 29-39, wherein the second polynucleotide comprises a promoter comprising a binding site for a transcription modulator that modulates the expression of the therapeutic agent in the cell.
41. The modified cell of embodiment 40, wherein the transcription modulator is or includes Hif1a, NFAT, FOXP3, and/or NFkB.
42. A method of inducing or causing or enhancing T cell response, treating cancer, or enhancing cancer treatment, the method comprising: administrating to a subject in need thereof an effective amount of the composition of the modified cells of any one of embodiments 29-41.
43. An isolated polynucleotide encoding a binding molecule comprising an extracellular domain, a transmembrane domain, and an intracellular domain, the extracellular domain binds an antigen, the intracellular domain comprising a cytoplasmic domain of a receptor of a therapeutic agent. Examples of the isolated polynucleotide are listed in Table 4.
44. A cell comprising the isolated polynucleotide of embodiment 43.
45. A pharmaceutical composition comprising the population of the cells of embodiment 44.
46. A method of inducing or causing T cell response in a subject in need thereof and/or treating a tumor of the subject, the method comprising administering an effective amount of the composition of embodiment 45 to the subject.
47. A modified cell comprising a binding molecule comprising an extracellular domain, a transmembrane domain, and an intracellular domain, the extracellular domain binds an antigen, the intracellular domain comprising a cytoplasmic domain of a receptor of a therapeutic agent.
48. The isolated polynucleotide and the modified cell of any one of embodiments 43-47, wherein the therapeutic agent is a cytokine.
49. The isolated polynucleotide and the modified cell of any one of embodiments 43-47, wherein the receptor of the therapeutic agent is or comprises IL12Rβ2, IL18R1, IL123R, GP130, IL15Ra, or IL12Rβ1.
50. The isolated polynucleotide and the modified cell of any one of embodiments 43-47, wherein the therapeutic agent is or comprises IFNγ, IL-2, IL-6, IL-7, IL-12, IL-15, IL-17, IL-18, or IL-23.
51. The isolated polynucleotide and the modified cell of any one of embodiments 43-47, wherein the cytoplasmic domain of the receptor is or comprises at least one of the amino acid sequences of SEQ ID NOs: 314-319.
52. The isolated polynucleotide and the modified cell of any one of embodiments 43-47, wherein the modified cell comprises the isolated polynucleotide comprising any one of the amino acid sequences of SEQ ID NOs: 320-322.
53. The isolated polynucleotide and the modified cell of any one of embodiments 43-51, wherein the modified cell comprises an additional polynucleotide, the isolated polynucleotide comprises additional polynucleotide, and the additional polynucleotide encodes 4-1BB domain and CD3 Zeta domain, a polynucleotide encoding the cytoplasmic domain of the receptor is located between the 4-1BB domain and CD3 zeta domain.
54. The isolated polynucleotide and the modified cell of any one of embodiments 43-51, wherein the modified cell comprises an additional polynucleotide, the isolated polynucleotide comprises additional polynucleotide, and the additional polynucleotide encodes 4-1BB domain and CD3 Zeta domain, a polynucleotide encoding the cytoplasmic domain of the receptor is located before the 4-1BB domain ordered from a N-terminal of the cytoplasmic domain.
55. The isolated polynucleotide and the modified cell of any one of embodiments 43-51, wherein the modified cell comprises an additional polynucleotide, the isolated polynucleotide comprises an additional polynucleotide, and the additional polynucleotide encodes a 4-1BB domain and a CD3 Zeta domain, a polynucleotide encoding the cytoplasmic domain of the receptor is located after the CD3 Zeta domain ordered from a N-terminal of the cytoplasmic domain.
56. The isolated polynucleotide and the modified cell of any one of embodiments 43-55, wherein the intracellular domain comprises a co-stimulatory domain that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.
57. The isolated polynucleotide and the modified cell of embodiment 56, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
58. The modified cell or the cell of any one of embodiments 44-57, wherein a signaling pathway is activated when the binding molecule binds to the antigen.
59. An isolated polynucleotide comprising a first polynucleotide and a second or additional polynucleotide, the first polynucleotide encoding a chimeric antigen receptor (CAR), the second or additional polynucleotide encoding a therapeutic agent that is or comprises at least one of IFNγ, IL-2, IL-6, IL-7, IL-12, IL-15, IL-17, IL-18, or IL-23.
60. An isolated polynucleotide comprising a first polynucleotide and a second or additional polynucleotide, the first polynucleotide encoding a chimeric antigen receptor (CAR), the second or additional polynucleotide encoding a therapeutic agent that is or comprises at least one of TNFRSF superfamily member receptor activation antibodies or membrane-bound forms thereof, TNFRSF superfamily member ligands or the membrane-bound form thereof, chemokines or membrane-bound forms thereof, antibodies to the chemokines, or antibodies to receptors of the chemokines or the membrane-bound forms thereof, and D28 family's ligands that correspond to the sequences in Table 2-4.
61. A population of CAR cells comprising the polynucleotide and the additional polynucleotide of embodiments 59 or 60, wherein the CAR cells comprise lymphocyte, leukocyte, or PBMC.
62. The population of CAR cells of embodiment 61, wherein the CAR and the therapeutic agent are produced in the form of a polyprotein, which is cleaved to generate separate CAR and therapeutic agent molecules.
63. The population of CAR cells of embodiment 61 or 62, wherein the polyprotein comprises a cleavable moiety between the CAR and the therapeutic agent, the cleavable moiety comprises a 2A peptide, the 2A peptide comprises P2A or T2A, and/or the CAR and the therapeutic agent are each constitutively expressed.
64. The population of CAR cells of any one of embodiments 61-63, wherein the CAR cells comprise: a third polynucleotide encoding an additional CAR binding to an antigen that is different from the CAR, or the additional CAR binding a solid tumor antigen, and the CAR binds an antigen of a white blood cell.
65. A pharmaceutical composition comprising the population of the CAR cells of any one of embodiments 61-64.
66. A method of causing or inducing T cell response in a subject in need thereof and/or treating a tumor of the subject, the method comprising administering an effective amount of the composition of embodiment 65 to the subject.
67. A modified cell comprising one or more CARs, wherein the cell is engineered to express and secrete a therapeutic agent that is or comprises at least one of IFNγ, IL-2, IL-6, IL-7, IL-12, IL-15, IL-17, IL-18, or IL-23.
68. A use of the composition of T cells comprising one or more CARs for causing, inducing, or enhancing T cell response, treating cancer, or enhancing cancer treatment, comprising: administering an effective amount of the composition of T cells comprising one or more CARs, wherein the cell is engineered to express and secrete a therapeutic agent that is or comprises at least one of IFNγ, IL-2, IL-6, IL-7, IL-12, IL-15, IL-17, IL-18, IL-23, or a combination thereof, and the T cell response is enhanced as compared to administering a composition of T cells that do not express or secrete the therapeutic agent.
69. A use of the composition of T cells comprising one or more CARs forcausing, inducing, or enhancing T cell response, treating cancer, or enhancing cancer treatment comprising: administering an effective amount of the composition of a population of T cells comprising a CAR; and administering an effective amount of a therapeutic agent that is or comprises at least one of IFNγ, IL-2, IL-6, IL-7, IL-12, IL-15, IL-17, IL-18, IL-23, ora combination thereof, wherein the T cell response is enhanced as compared to the administration of CAR T cells without the administration of therapeutic agent.
70. The use of embodiment 69, wherein the administering the effective amount of the therapeutic agent comprises intravenous delivery of an amount of human IL-6 in the range of about 0.5-50 ug per kilogram of body weight.
71. The use of any one of embodiments 67-70, wherein the T cell comprises an additional CAR binding a solid tumor antigen, and the CAR binds an antigen of a white blood cell.
72. The use of embodiment 71, wherein the solid tumor antigen is tMUC1, PRLR, CLCA1, MUC12, GUCY2C, GPR35, CR1L, MUC17, TMPRSS11B, MUC21, TMPRSS11 E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the B cell antigen is CD19, CD20, CD22, or BCMA.
73. The isolated polynucleotide, the modified cell, or the use of any one of embodiments 60-72, wherein the therapeutic agent is IL-6 or IL-7.
74. The isolated polynucleotide, the modified cell, or the use of any one of embodiments 60-72, wherein the CAR comprises an extracellular domain, a transmembrane domain, and an intracellular domain, the extracellular domain binds an antigen.
75. The isolated polynucleotide, the modified cell, or the use of embodiment 74, wherein the intracellular domain comprises a co-stimulatory domain that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.
76. The isolated polynucleotide, the modified cell, or the use of embodiment 75, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
77. The isolated polynucleotide, the modified cell, or the use of any one of embodiments 60-76, wherein the modified cell or T cells comprise a dominant negative form of PD-1 mutant such that PD-1/PDL-1 signaling pathway of the cell is interfered or blocked.
78. The isolated polynucleotide, the modified cell, or the use of any one of embodiments 60-77, wherein the therapeutic agent is present in the modified cell in a recombinant DNA construct, in an mRNA, or in a viral vector.
79. The isolated polynucleotide, the modified cell, or the use of any one of embodiments 60-77, wherein the modified cell comprises a therapeutic agent mRNA encoding the therapeutic agent, and the mRNA is not integrated into the genome of the modified cell.
80. The isolated polynucleotide, the modified cell, or the use of any one of embodiments 60-79, wherein the therapeutic agent corresponds to at least one of sequence listed in Table 2-4.
81. The isolated polynucleotide, the modified cell, or the use of any one of embodiments 60-79, wherein the modified cell comprises a polynucleotide comprising a promoter comprising a binding site for a transcription modulator that modulates the expression of the therapeutic agent in the cell.
82. The isolated polynucleotide, the modified cell, or the use of embodiment 81, wherein the transcription modulator is or includes Hif1a, NFAT, FOXP3, and/or NFkB.
83. The isolated polynucleotide, the modified cell, or the use of embodiment 82, wherein the promoter is responsive to the transcription modulator.
84. The isolated polynucleotide, the modified cell, or the use of embodiment 83, wherein the promoter is operably linked to the polynucleotide encoding the therapeutic agent such that the promoter drives expression of the therapeutic agent in the cell.
85. The isolated polynucleotide, the modified cell, or the use of embodiment 81, wherein the promoter and the binding site correspond to the sequences listed in Table 2-4.
86. A modified cell comprising one or more CARs, wherein the cell is engineered to express and secrete a therapeutic agent such as a cytokine, and wherein examples of the modified cell are shown in FIGS. 5-10, and 55, 56.
87. A method of causing, inducing, or enhancing T cell response, treating cancer, or enhancing cancer treatment, the method comprising: administrating an effective amount of the composition of T cells comprising one or more CARs, wherein the cell is engineered to express and secrete a therapeutic agent such as a cytokine.
88. The modified cell or the method of embodiment 86 or 87, wherein the therapeutic agent that is or comprises IFN-γ.
89. The modified cell or the method of any one of embodiments 86-88, wherein the therapeutic agent that is or comprises IL-6, IFN-γ, or a combination thereof, wherein the therapeutic agent comprises SEQ ID NO: 287 or 328.
90. The modified cell or the method of any one of embodiments 86-89, wherein the therapeutic agent that is or comprises IL-15, IL-12, or a combination thereof.
91. The modified cell or the method of any one of embodiments 86-90, wherein the small protein or the therapeutic agent is or comprises a recombinant or native cytokine.
92. The modified cell or the method of any one of embodiments 86-91, wherein the therapeutic agent comprises a FC fusion protein associated with a small protein.
93. The modified cell or the method of any one of embodiments 86-92, wherein the small protein is or comprises IL-12, IL-15, IL-6, IFN-γ, or a combination thereof.
94. The modified cell or the method of any one of embodiments 86-93, wherein expression and/or secretion is regulated by a controlling system such as an inducible expression system, or the modified cell is regulated by an inducible suicide expression.
95. The modified cell or the method of any one of embodiments 86-94, wherein the therapeutic agent activates macrophages and/or dendritic cells.
96. The modified cell or the method of any one of embodiments 86-95, wherein the therapeutic agent causes or promotes macrophages to remove granulocytes.
97. The modified cell or the method of any one of embodiments 86-96, wherein the therapeutic agent inhibits or suppresses growth of cancer cells.
98. The modified cell or the method of any one of embodiments 86-97, wherein the therapeutic agent is or comprises a recombinant or a native protein.
99. The modified cell or the method of any one of embodiments 86-98, wherein the modified cell comprises a modified programmed cell death protein 1 (PD-1) that is a dominant negative form of PD-1.
100. The modified cell or the method of any one of embodiments 86-99, wherein the one or more CARs comprise a CAR targeting a tumor cell.
101. The modified cell or the method of any one of embodiments 86-100, wherein the one or more CARs comprise a CAR binding a solid tumor antigen and an additional CAR binding a blood cell antigen such as a B cell antigen.
102. The modified cell or the method of any one of embodiments 86-100, wherein the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, or EGFR, and the B cell antigen is CD19, CD20, CD22, or BCMA.
103. The modified cell or the method of any one of embodiments 86-102, wherein the solid tumor antigen comprises B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2, and the B cell antigen is CD19, CD20, CD22, or BCMA.
104. The modified cell or the method of any one of embodiments 86-103, wherein the T cell response in a subject administered with the CAR T cells expressing the therapeutic agent is enhanced as compared to the the T cell response in a subject that is administered with CAR T cells that do not express or secrete the therapeutic agent.
105. The modified cell or the method of any one of embodiments 86-104, wherein the modified cell comprises a polynucleotide encoding hTERT or a nucleic acid encoding SV40LT, or a combination thereof, wherein the polynucleotide encoding hTERT or a nucleic acid encoding SV40LT, or a combination thereof is integrated into the genome of the modified T cell, and the modified T cell constitutively expresses hTERT, SV40LT, or a combination thereof.
106. The modified cell or the method of any one of embodiments 86-105, wherein expression of the polynucleotide encoding hTERT, a nucleic acid encoding SV40LT, or a combination thereof, is regulated by an inducible expression system, and/or the modified T cell comprises a polynucleotide encoding a suicide gene.
107. The modified cell or the method of any one of embodiments 86-106, wherein the modified cell is derived from a healthy donor or the subject.
108. The modified cell or the method of any one of embodiments 86-107, wherein the TRAC gene of the modified cell is inactivated.
109. The modified cell or the method of any one of embodiments 86-108, wherein the modified cell has a reduced graft-versus-host disease (GVHD) response in a bioincompatible human recipient as compared to the GVHD response of the primary human T cell in response to allogenic CAR T treatment.
110. The modified cell or the method of any one of embodiments 86-109, wherein the modified cell has reduced amount PD-1 or has a dominant negative form of PD-1 such that an signaling pathway of the PD-1 is blocked.
111. The modified cell or the method of any one of embodiments 86-110, wherein the modified cell has reduced amount PD-1 or has a dominant negative form of PD-1 such that an signaling pathway of the PD-1 is blocked, the therapeutic agent is IL-12 or IFN-γ, or a combination thereof.
112. The modified cell or the method of an embodiments 86-111, wherein the modified cell has reduced amount PD-1 or has a dominant negative form of PD-1 such that an signaling pathway of the PD-1 is blocked, the therapeutic agent comprises a CD40 agonist such as CP-870,893 (from Pfizer).
113. The modified cell or the method of an embodiments 86-112, wherein the modified cell comprises an additional therapeutic agent, the modified cell comprises a polynucleotide encoding the therapeutic agent and an additional polynucleotide encoding the additional therapeutic agent, and the polynucleotide and the additional polynucleotide are connected by an IRES element or a third polynucleotide encoding a 2A peptide.
114. The modified cell or the method of embodiment 113, wherein the therapeutic agent is IL-6, and the additional therapeutic agent is IFN-γ.
115. The modified cell or the method of embodiment 113, wherein the therapeutic agent is IL-12, and the additional therapeutic agent is IFN-γ.
116. The modified cell or the method of embodiment 113, wherein the therapeutic agent is CD40, and the additional therapeutic agent is IFN-γ.
117. The modified cell or the method of an embodiments 86-116, wherein the modified cell comprises a polynucleotide comprising a promoter comprising a binding site for a transcription modulator that modulates the expression and/or secretion of the therapeutic agent in the cell.
118. The modified cell or the method of any one of embodiments 86-116, wherein the transcription modulator is or includes Hif1a, NFAT, FOXP3, and/or NFkB.
119. The modified cell or the method of embodiment 118, wherein the promoter is responsive to the transcription modulator.
120. The modified cell or the method of any one of embodiments 118-119, wherein the promoter is operably linked to the polynucleotide encoding the therapeutic agent such that the promoter drives expression and/or secretion of the therapeutic agent in the cell.
121. The modified cell or the method of any one of embodiments 118-120, wherein the promoter comprises at least one of SEQ ID NOs: 323-325.
122. The modified cell or the method of any one of embodiments 86-120, wherein the modified cell comprises one or more polynucleotides encoding a stimulus response element and encoding one or more CARs and/or the therapeutic agent, and the stimulus response element comprises at least one portion of the cGMP-specific 3′,5′-cyclic phosphodiesterase or a molecule derived of, for example, SEQ ID NO: 329.
123. The modified cell or the method of embodiment 122, wherein expression of the one or more CARs and/or the therapeutic agent is ligand dependent.
124. The modified cell or the method of embodiment 122, wherein the one or more CARs and/or the therapeutic agent are destabilized or degraded in the absence of a corresponding ligand.
125. The modified cell or the method of any one of embodiments 122-124, wherein modified cell comprises one or more polynucleotides encoding at least one portion of the cGMP-specific 3′,5′-cyclic phosphodiesterase or a molecule derived thereof, for example, SEQ ID NO: 329 appended to or associated with the therapeutic agent such that expression of the therapeutic agent is ligand dependent, and the therapeutic agent is or comprises IL6 or IFN-γ, or a combination thereof.
126. A fusion protein comprising a scFv binding PD-1 or PDL1, a linker, an extracellular domain, a transmembrane domain, and a cytoplasmic domain, wherein the transmembrane domain is selected from the group consisting of a transmembrane domain of a receptor of I15, IL2, IL7, IL6, IL12, IL18, IL21, IL23, IL33, TNFα, TNFβ, IFNα, IFNγ, IFNβ, and siglec-15 antigen, and the cytoplasmic domain is selected from the group consisting of a cytoplasmic domain of receptor of the receptor of I15, IL2, IL7, IL6, IL12, IL18, IL21, IL23, IL33, TNFα, TNFβ, IFNα, IFNγ, IFNβ, and siglec-15 antigen, and the extracellular domain is selected from the group consisting of an extracellular domain of the receptor of I15, IL2, IL7, IL6, IL12, IL18, IL21, IL23, IL33, TNFα, TNFβ, IFNα, IFNγ, IFNβ, and siglec-15 antigen.
127. A fusion protein comprising a scFv binding PD-1 or PDL1, a linker, a transmembrane domain, and a cytoplasmic domain, wherein the transmembrane domain is selected from the group consisting of a transmembrane domain of a receptor of CD4, CD8, CD28, CD27, CD25, CD137, PD1, PDL1, and siglec-15 antigen, and the cytoplasmic domain is selected from the group consisting of a cytoplasmic domain of receptor of the receptor of CD4, CD8, CD28, CD27, CD25, CD137, PD1 PDL1, and siglec-15 antigen.
128. A fusion protein comprising a cytokine is selected from the group consisting of IL15, IL2, IL7, IL6, IL12, IL18, IL21, IL23, IL33, TNFα, TNFβ, IFNα, and IFNβ, wherein the transmembrane domain is selected from the group consisting of a transmembrane domain of the receptor of IL15, IL2, IL7, IL6, IL12, IL18, IL21, IL23, IL33, TNFα, TNFβ, IFNα, IFNγ, IFNβ, and siglec-15 antigen, and the cytoplasmic domain is selected from the group consisting of a cytoplasmic domain of receptor of the receptor of IL15, IL2, IL7, IL6, IL12, IL18, IL21, IL23, IL33, TNFα, TNFβ, IFNα, and IFNβ, and the extracellular domain is selected from the group consisting of an extracellular domain of the receptor of IL15, IL2, IL7, IL6, IL12, IL18, IL21, IL23, IL33, TNFα, TNFβ, IFNα, and IFNβ.
129. A fusion protein comprising a cytokine is selected from the group consisting of IL15, IL2, IL7, IL6, IL12, IL18, IL21, IL23, IL33, TNFα, TNFβ, IFNα, and IFNβ, wherein the transmembrane domain is selected from the group consisting of a transmembrane domain of a receptor of CD4, CD8, CD28, CD27, CD25, CD137, PD1, PDL1, and siglec-15 antigen, and the cytoplasmic domain is selected from the group consisting of a cytoplasmic domain of receptor of the receptor of CD4, CD8, CD28, CD27, CD25, CD137, PD1, PDL1, and siglec-15 antigen. Examples of the fusion protein are shown in FIG. 4.
130. A fusion protein comprising a binding domain binding a ligand or a receptor of an immune checkpoint molecule and a docking molecule, wherein the immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), VISTA (its receptor), and CD 160, and the docking molecule associates the binding domain with a cell.
131. The fusion protein of embodiment 130, wherein the docking molecule comprises a linker, a transmembrane domain, and a cytoplasmic domain.
132. The fusion protein of embodiment 131, wherein the transmembrane domain is selected from the group consisting of a transmembrane domain of a receptor of I15, IL2, IL7, IL6, IL12, IL18, IL21, IL23, IL33, TNFα, TNFβ, IFNα, and IFNβ, and the cytoplasmic domain is selected from the group consisting of a cytoplasmic domain of receptor of the receptor of I15, IL2, IL7, IL6, IL12, IL18, IL21, IL23, IL33, TNFα, TNFβ, IFNα, and IFNβ.
133. The fusion protein of embodiment 131, wherein the docking molecule further comprises an extracellular domain.
134. The fusion protein of embodiment 133, wherein the extracellular domain is selected from the group consisting of an extracellular domain of the receptor of I15, IL2, IL7, IL6, IL12, IL18, IL21, IL23, IL33, TNFα, TNFβ, IFNα, and IFNβ.
135. The fusion protein of embodiment 130, wherein the docking molecule comprises a linker, a transmembrane domain, and a cytoplasmic domain.
136. The fusion protein of embodiment 135, wherein the transmembrane domain is selected from the group consisting of a transmembrane domain of a receptor of CD4, CD8, CD28, CD27, CD25, CD137, PD1, PDL1, and siglec-15 antigen, and the cytoplasmic domain is selected from the group consisting of a cytoplasmic domain of receptor of the receptor of CD4, CD8, CD28, CD27, CD25, CD137, PD1, PDL1, and siglec-15 antigen.
137. The fusion protein of any one of embodiments 130-136, wherein the binding domain is a scFv.
138. The fusion protein of embodiment 130, wherein the binding domain is a scFv binding CD80 or CD86.
139. The fusion protein of embodiment 138, wherein the docking molecule comprises or is a wild type or modified CTLA4 or PD-1.
140. The fusion protein of embodiment 130, wherein the binding domain is a scFv binding VISTA.
141. The fusion protein of embodiment 140, wherein the docking molecule comprises or is a wild type or modified VISTA receptor or PD-1.
142. The fusion protein of embodiment 130, wherein the binding domain is a scFv binding PDL1 or PD1, and/or the docking molecule comprises or is wide type or modified PD-1.
143. The fusion protein of embodiment 130, wherein the binding domain is a scFv binding B7-H3.
144. The fusion protein of embodiment 143, wherein the docking molecule comprises or is wild type or modified B7-H3 receptor or PD-1.
145. A fusion protein comprising a therapeutic agent and a docking molecule, wherein the docking molecule comprises a cytoplasmic domain and a transmembrane domain that associate the therapeutic agent with a cell.
146. The fusion protein of embodiment 142, wherein the therapeutic agent comprises or is the binding domain of any one of embodiment 126-145 or the cytokine of embodiment 129 or 130.
147. A polynucleotide encoding the fusion protein of any one of embodiments 126-146.
148 A modified cell comprising the fusion protein of any one of embodiments 126-148 or the polynucleotide of embodiment 147.
149. A pharmaceutical composition comprising the population of the modified cells of embodiment 148.
150. A method of causing or inducing T cell response in a subject in need thereof and/or treating a tumor of the subject, the method comprising administering an effective amount of the composition of embodiment 148 to the subject.
151. The pharmaceutical composition, the modified cell, and the method of any one of embodiments 148-150, wherein the docking molecule comprises a linker and the linker is a GS linker.
152. The pharmaceutical composition, the modified cell, and the method of any one of embodiments 148-150, wherein the modified cell comprises a CAR.
153. The pharmaceutical composition, the modified cell, and the method of any one of embodiments 148-150, wherein the CAR comprises an extracellular domain, a transmembrane domain, and an intracellular domain, the extracellular domain binds an antigen.
154. The pharmaceutical composition, the modified cell, and the method of any one of embodiments 148-150, wherein the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.
155. The pharmaceutical composition, the modified cell, and the method of any one of embodiments 148-26, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
156. The pharmaceutical composition, the modified cell, and the method of any one of embodiments 151-156, wherein the fusion protein is regulated by an inducible gene expression system.
157. A fusion protein comprising a cytokine and an oxygen-sensitive polypeptide domain.
158. The fusion protein of embodiment 157, wherein the oxygen-sensitive polypeptide domain is HIFI alpha, HIF3 alpha, or a polypeptide comprising an amino acid sequence having a sequence identity of over 80%, 90% or 95% with respectively Hif VHL-interaction domain, Hif amino acid 344-417, or Hif amino acid 380-603.
159. The fusion protein of embodiment 158, wherein the oxygen-sensitive polypeptide domain comprises HIF VHL binding domain.
160. The fusion protein of embodiment 157, wherein HIF1 alpha is hydroxylated by HIFa specific prolyl hydroxylases (PHDI-3) which are oxygen sensing.
161. A polynucleotide encoding the fusion of any of embodiments 157-160 or comprising one or more components shown in FIGS. 5-10.
162. A modified cell comprising the fusion protein of any of embodiments 157-160 and/or the nuclei acid sequence of embodiments 161.
163. The modified cell of embodiment 162, wherein the fusion protein is regulated by NFAT.
164. A pharmaceutical composition comprising a population of the modified cells of embodiment 162 or 163.
165. A method of causing or inducing T cell response in a subject in need thereof and/or treating a tumor of the subject, the method comprising administering an effective amount of the composition of embodiment 164 to the subject.
166. The modified cell, pharmaceutical composition, or method of any one of embodiments 162-165, wherein the modified cell is lymphocyte, leukocyte, or PBMC; or cells, NK cells, or dendritic cells.
167. The modified cell, pharmaceutical composition, or method of any one of embodiments 162-166, wherein the modified cell further comprises a Chimeric antigen receptor (CAR) or a modified TCR.
168. The modified cell, pharmaceutical composition, or method of embodiment 167, wherein the TCR is modified TCR.
169. The modified cell, pharmaceutical composition, or method of embodiment 167, wherein the TCR is derived from spontaneously occurring tumor-specific T cells in patients.
170. The modified cell, pharmaceutical composition or method of embodiment 167, wherein the TCR binds a tumor antigen.
171. The modified cell, pharmaceutical composition or method of embodiment 170, wherein the tumor antigen comprises CEA, gp100, MART-1, p53, MAGE-A3, or NY-ESO-1, or the TCR comprises TCRγ and TCRδ Chains or TCRα and TCRβ chains, or a combination thereof.
172. The modified cell, pharmaceutical composition or method of embodiment 167, wherein the CAR comprises an extracellular domain, a transmembrane domain, and an intracellular domain, the extracellular domain binding an antigen.
173. The modified cell, pharmaceutical composition or method of embodiment 172, wherein the intracellular domain comprises a co-stimulatory domain that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.
174. The modified cell, pharmaceutical composition or method of embodiment 173, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
175. The modified cell, pharmaceutical composition or method of any one of embodiments 162-174, wherein the modified cell or the T cells comprise an additional CAR binding a solid tumor antigen, and the (first) CAR binds an antigen of a white blood cell.
176. The modified cell, pharmaceutical composition or method of any one of embodiments 162-174, wherein the modified cell or the T cells comprise a dominant negative form of PD-1.
177. The modified cell, pharmaceutical composition or method of any one of embodiments 162-174, wherein the modified cell or the T cells comprise a modified PD-1 lacking a functional PD-1 intracellular domain.
178. The modified cell, pharmaceutical composition, or method of any one of embodiments 162-177, wherein the modified cell further comprises a polynucleotide encoding a therapeutic agent.
179. The modified cell, pharmaceutical composition, or method of any one of embodiments 178, wherein the isolated polynucleotide comprises a promoter comprising a binding site for a transcription modulator that modulates the expression and/or secretion of the therapeutic agent in the cell.
180. The modified cell, pharmaceutical composition, or method of embodiment 179, wherein the transcription modulator is or includes Hif1a, NFAT, FOXP3, and/or NFkB.
181. The modified cell, pharmaceutical composition, or method of embodiment 180, wherein the promoter is responsive to the transcription modulator.
182. The modified cell, pharmaceutical composition, or method of embodiment 180, wherein the promoter is operably linked to the polynucleotide encoding the therapeutic agent such that the promoter drives expression and/or secretion of the therapeutic agent in the cell.
183. The modified cell, pharmaceutical composition, or method of embodiment 180, wherein expression of the therapeutic agent is regulated by an inducible gene expression system.
184. The modified cell, pharmaceutical composition, or method of embodiment 183, wherein the inducible gene expression system comprises or is a lac system, a tetracycline system, or a galactose system.
185. The modified cell, pharmaceutical composition or method of embodiment 183, wherein the inducible gene expression system comprises or is a tetracycline system.
186. The modified cell, pharmaceutical composition or method of embodiment 185, wherein the inducible gene expression system comprises or is a tetracycline on system, and an inducer of the inducible gene expression system is tetracycline, doxycycline, or an analog thereof.
187. The modified cell, pharmaceutical composition, or method of any one of embodiments 162-186, wherein the modified cell is a T cell derived from a primary human T cell isolated from a human donor.
188. The modified cell, pharmaceutical composition, or method of embodiment 187, wherein the cell has a reduced expression of endogenous TRAC gene.
189. The modified cell, pharmaceutical composition, or method of any one of embodiments 162-186, wherein the modified cell is a T cell derived from a primary human T cell isolated from a subject having cancer.
190. A composition comprising a first population of cells comprising a first molecule binding a first antigen and a second population of cells comprising a second molecule binding a second antigen, wherein the second antigen is a tumor antigen and the first antigen and second antigen are different antigens, and the first population of cells and/or the second population of cells comprise a polynucleotide encoding a therapeutic agent that is or comprises IL-6 or IFN-γ, or a combination thereof.
191. The composition of embodiment 190, wherein the first molecule is a first CAR, and the second molecule is a second CAR; or the first molecule is the first CAR, and the second molecule is a TCR.
192. The composition of embodiment 191, wherein the first population of cells does not comprise the second CAR, and/or the second population of cells does not comprise the first CAR.
193. The composition of embodiment 192, wherein the composition further comprises a third population of cells comprising one or more polynucleotides encoding the first CAR and the second CAR.
194. The composition of embodiment 191, wherein: the second population of cells comprises the first CAR, and the first population of cells do not comprise the second CAR; or the first population of cells comprises the second CAR.
195. The composition of embodiment 194, wherein second population of cells does not comprise the first CAR, and the first population of cells comprise the second CAR.
196. A method of enhancing expansion of the second population of cells (cells targeting solid tumor and/or lymphoma), the method comprising administering an effective amount of the composition of any one of embodiments 190-195 to a subject having a form of cancer associated with or expresses a tumor antigen.
197. A method of enhancing T cell response in a subject or treating the subject having cancer, the method comprising administering an effective amount of the composition of any one of embodiments 190-195 to the subject having cancer associated with or expresses a tumor antigen.
198. A method of enhancing expansion of cells in a subject, the method comprising: contacting cells with a first vector comprising a first polynucleotide encoding the first CAR and a second vector comprising a second polynucleotide encoding the second CAR to obtain the composition of any one of embodiments 190-195; and administering an effective amount of the composition to the subject having a form of cancer associated with or expresses a tumor antigen.
199. A method of enhancing T cell response in a subject or treating the subject having cancer, the method comprising: introducing into cells with a first vector comprising a first polynucleotide encoding the first CAR and a second vector comprising a second polynucleotide encoding the second CAR to obtain the composition of any one of embodiments 190-195; and administering an effective amount of the composition to the subject having a form of cancer associated with or expresses the tumor antigen.
200. A method of enhancing expansion of cells in a subject, the method comprising: administering an effective amount of the first population of cells of any one of embodiments 190-195; and administering an effective amount of the second population of cells.
201. The method of any one of embodiments 198-200, wherein the first vector and the second vector comprise lentiviral vectors.
202. The composition or the method of any one of embodiments 190-201, wherein the first or second antigen is or comprises a surface molecule of a white blood cell (WBC), a tumor antigen, or a solid tumor antigen.
203. The composition or the method of any one of embodiments 190-201, wherein the cells are modified T cells, modified NK cells, or modified dendritic cells.
204. The composition or the method of embodiment 202, wherein the WBC is a granulocyte, a monocyte, or lymphocyte.
205. The composition or the method of embodiment 204, wherein the WBC is a B cell.
206. The composition or the method of embodiment 205, wherein the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13.
207. The composition or the method of embodiment 202, wherein the cell surface molecule of the WBC is CD19, CD20, CD22, or BCMA.
208. The composition or the method of embodiment 202, wherein the cell surface molecule of the WBC is CD19.
209. The composition or the method of embodiment 202, wherein the tumor antigen is a solid tumor antigen.
210. The composition or the method of embodiment 202, wherein the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, or EGFR.
211. The composition or the method of embodiment 202, wherein the solid tumor antigen is or comprises tumor associated MUC1.
212. The composition or the method of any one of embodiments 191-211, wherein the CAR comprises the antigen binding domain, a transmembrane domain, a co-stimulatory domain, and a CD3 zeta domain.
213. The composition or the method of embodiment 212, wherein the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or a combination thereof.
214. The composition or the method of embodiment 213, wherein: a co-stimulatory domain of the second CAR comprises or is an intracellular domain of 4-1 BB, and a binding domain of the second CAR binds TSHR; and/or a binding domain of the first CAR binds CD19 and a co-stimulatory domain of the second CAR comprises or is an intracellular domain of CD28.
215. The composition or the method of any one of embodiments 191-214, wherein the first population of cells and/or the second population of cells further comprise a dominant negative form of PD-1.
216. The composition or the method of embodiment 215, wherein the first population of cells comprise a vector encoding the first CAR and the dominant negative form of PD-1.
217. The composition or the method of any one of embodiments 191-216, wherein the first CAR comprises a scFv binding TSHR, an intracellular domain of 4-1BB or CD28, and a CD3 zeta domain, and the second CAR comprises a scFv binding CD19, an intracellular domain of 4-1 BB or CD28, and a CD3 zeta domain.
218. The composition or the method of any one of embodiments 191-217, wherein the first CAR comprises SEQ ID NO: 5, and the second CAR comprises SEQ ID NO: 70.
219. The composition or the method of any one of embodiments 191-218, wherein the second population of cells comprises a lentiviral vector encoding the first CAR and a therapeutic agent and the first population of cells comprises a lentiviral vector encoding the second CAR and a dominant negative form of PD-1.
220. The composition or the method of any one of embodiments 191-219, wherein the first population of cells comprises the first CAR and a therapeutic agent and the second population of cells comprises the second CAR and a dominant negative form of PD-1.
221. The composition or the method of any one of embodiments 219 and 220, wherein the therapeutic agent comprises or is a cytokine.
222. The composition or the method of embodiment 221, wherein the cytokine is IL6 and/or INFy.
223. A method comprising:
administering an effective amount of a first population of T cells comprising a CAR comprising a scFv binding CD19, an intracellular domain of 4-1BB or CD28, CD3 zeta domain to the patient, thereby enhancing expansion of the first population of T cells in the patient; and
administering an effective amount of a second population of T cells comprising a CAR comprising a scFv binding TSHR to a patient having cancer, an intracellular domain of 4-1BB or CD28, and a CD3 zeta domain.
   224. The method of embodiment 223, wherein first population of cells further comprise an additional CAR comprising the scFv binding tMUC1, the intracellular domain of 4-1BB or CD28, and the CD3 zeta domain.
   225. The method of embodiment 223, wherein the second population of cells does not comprise the scFv binding CD19.
   226. The method of embodiment 223, wherein the first population of cells does not comprise the scFv binding TSHR.
   227. The composition of embodiment 190, wherein the first molecule is a modified TCR.
   228. The composition of embodiment 227, wherein the TCR is derived from spontaneously occurring tumor-specific T cells in patients.
   229. The composition of embodiment 227, wherein the TCR binds a tumor antigen.
   230. The composition of embodiment 227, wherein the tumor antigen comprises CEA, gp100, MART-1, p53, MAGE-A3, or NY-ESO-1, or the TCR comprises TCRγ and TCRδ chains, or TCRα and TCRβ chains, or a combination thereof.
   231. The modified cell, pharmaceutical composition or method of any one of embodiments 190-230, wherein the modified cell is a T cell derived from a primary human T cell isolated from a human donor.
   232. The modified cell, pharmaceutical composition, or method of embodiment 231, wherein the cell has a reduced expression of endogenous TRAC gene.
   233. The modified cell, pharmaceutical composition or method of any one of embodiments 190-230, wherein the modified cell is a T cell derived from a primary human T cell isolated from a subject having cancer.
   234. The isolated polynucleotide, the population of CAR cells, the pharmaceutical composition, or the method of any one of embodiments 190-233, wherein the polynucleotide comprises a first polynucleotide encoding IL6 and a second polynucleotide encoding IFN-γ, and the first polynucleotide and the second polynucleotide are connected by an IRES element or a third polynucleotide encoding a 2A peptide.
   235. The isolated polynucleotide, the population of CAR cells, the pharmaceutical composition, or the method of any one of embodiments 190-234, wherein the polynucleotide is or comprises the polynucleotide encoding one or more amino acid sequences of SEQ ID NOs: 287 and/or 328, or a combination thereof.
   236. The isolated polynucleotide, the population of CAR cells, the pharmaceutical composition, or the method of any one of embodiments 190-235, wherein expression of the polynucleotide is regulated by a conditional expression system such that the therapeutic agent is expressed in response to binding of a target antigen; or the isolated polynucleotide, the population of CAR cells, the pharmaceutical composition, or the method of one of embodiments 1-47, wherein expression of the additional polynucleotide is regulated by SynNotch polypeptide.
   237. The isolated polynucleotide, the population of CAR cells, the pharmaceutical composition, modified cell, or the method of any one of embodiments 190-236, wherein T cell response in a subject administered with T cells expressing or secreting the therapeutic agent is enhanced as compared to the T cell response in a subject administered with T cells that do not express or secrete the therapeutic agent, or the T cell response in a subject administered with CAR T cells and the therapeutic agent is enhanced as compared with T cell response in a subject administered with CAR T cells without the administration of therapeutic agent.
   238. The isolated polynucleotide, the population of CAR cells, the pharmaceutical composition, modified cell, or the method of any one of embodiments 190-237, wherein expression and/or secretion of the therapeutic agent is regulated by an inducible expression system and/or the modified cell comprises a polynucleotide encoding an inducible suicide system.
   239. The isolated polynucleotide, the population of CAR cells, the pharmaceutical composition, modified cell, or the method of embodiments 190-49, wherein a range of concentration values of IL6 is 60 to 5000 μg/ml, 200-5000 μg/ml, or 2000-5000 μg/ml in the blood of the subject.
   240. The isolated polynucleotide, the population of CAR cells, the pharmaceutical composition, modified cell, or the method of any one of embodiments 190-239, wherein a range of concentration values IFN-γ is 20 to 5000 μg/ml, 200 to 5000 μg/ml, or 500 to 5000 μg/ml in the blood of the subject.
   241. The isolated polynucleotide, the population of CAR cells, the pharmaceutical composition, modified cell, or the method of any one of embodiments 190-240, wherein the modified cell comprises a polynucleotide comprising a binding site for a transcription modulator that modulates the expression and/or secretion of the therapeutic agent in the cell.
   242. The isolated polynucleotide, the population of CAR cells, the pharmaceutical composition, modified cell, or the method of embodiment 241, wherein the transcription modulator is or includes Hif1a, NFAT, FOXP3, and/or NFkB, and the promoter is responsive to the transcription modulator.
   243. The isolated polynucleotide, the population of CAR cells, the pharmaceutical composition, modified cell, or the method of embodiment 241, wherein the promoter is operably linked to the polynucleotide encoding the therapeutic agent such that the promoter drives 57 and/or secretion of the therapeutic agent in the cell.
   244. The isolated polynucleotide, the population of CAR cells, the pharmaceutical composition, modified cell, or the method of any one of embodiments 241-243, wherein the promoter comprises at least one of SEQ ID NOs: 332, 333, 341, 469, or 342.
   245. The isolated polynucleotide, the population of CAR cells, the pharmaceutical composition, modified cell, or the method of any one of embodiments 190-57, wherein the first and second population of cells comprises TSHR-CAR (scFv of the CAR: SEQ ID NO: 8): and hCD19-CAR-NATF-IL6-2A-IFNγ (scFv of CD19 CAR: SEQ ID 5; amino acid sequence (aa) of NATF: SEQ ID: 469; aa of IL6: SEQ ID NO: 287; 2A is SEQ ID NO: 327: and aa of IFN-γ: SEQ ID NO: 328).
   246. An isolated polynucleotide comprising a polynucleotide and an additional polynucleotide, the polynucleotide encoding a chimeric antigen receptor (CAR), the additional polynucleotide encoding a therapeutic agent that is or comprises IL-6 or IFN-γ, or a combination thereof.
   247. A population of CAR cells comprising the polynucleotide and the additional polynucleotide of embodiments 246, wherein the CAR cells comprise lymphocyte, leukocyte, or PBMC.
   248. The population of CAR cells of embodiment 247, wherein the CAR and the therapeutic agent are produced in the form of a polyprotein, which is cleaved to generate separate CAR and therapeutic agent molecules.
   249. The population of CAR cells of any one of embodiments 247-248, wherein the polyprotein comprises a cleavable moiety between the CAR and the therapeutic agent, the cleavable moiety comprises a 2A peptide, the 2A peptide comprises P2A or T2A, and/or the CAR and the therapeutic agent are each constitutively expressed.
   250. The population of CAR cells of any one of embodiments 247-249, wherein the CAR cells comprise:
a third polynucleotide encoding an additional CAR binding to an antigen that is different from an antigen that the CAR binds, or
the additional CAR binding a solid tumor antigen, and the CAR binds an antigen of a white blood cell.
   251. A pharmaceutical composition comprising the population of the CAR cells of any one of embodiments 247-250.
   252. A method of causing or inducing T cell response in a subject in need thereof and/or treating a tumor of the subject, the method comprising administering an effective amount of the composition of embodiment 251 to the subject.
   253. The isolated polynucleotide, the population of CAR cells, the pharmaceutical composition, or the method of any one of embodiments 246-252, wherein the additional polynucleotide comprises a first polynucleotide encoding IL6 and a second polynucleotide encoding IFN-γ, and the first polynucleotide and the second polynucleotide are connected by an IRES element or a third polynucleotide encoding a 2A peptide.
   254. The isolated polynucleotide, the population of CAR cells, the pharmaceutical composition, or the method of any one of embodiments 246-253, wherein the additional polynucleotide is or comprises the polynucleotide of SEQ ID NOs: 287 or 328, or a combination thereof.
   255. The isolated polynucleotide, the population of CAR cells, the pharmaceutical composition, or the method of any one of embodiments 246-254, wherein expression of the additional polynucleotide is regulated by a conditional expression system such that the therapeutic agent is expressed in response to binding of a target antigen, and/or wherein expression of the additional polynucleotide is regulated by SynNotch polypeptide.
   256. A modified cell comprises one or more CARs, wherein the cell is engineered to express and secrete a therapeutic agent that is or comprises IL-6 or IFN-γ, or a combination thereof.
   257. A method of causing, inducing, or enhancing T cell response, treating cancer, or enhancing cancer treatment, the method comprising: administrating an effective amount of a composition of T cells comprising one or more CARs, wherein the cell is engineered to express and secrete a therapeutic agent that is or comprises IL-6 or IFN-γ, or a combination thereof.
   258. A method of causing, inducing, or enhancing T cell response, treating cancer, or enhancing cancer treatment, the method comprising:
administering an effective amount of the composition of a population of T cells comprising a CAR; and
administering an effective amount of a therapeutic agent that is or comprises IL-6 or IFN-γ, or a combination thereof.
   259. The modified cell or the method of any one of embodiments 252, 257, and 258, wherein T cell response is enhanced in the subject administered with T cells that express or secrete the therapeutic agent as compared to the T cell response in the subject administered with T cells that do not express or secrete the therapeutic agent, or the T cell response is enhanced in the subject administered with CAR T cells and the therapeutic agent as compared to the T cell response in the subject administered with CAR T cells without the administration of therapeutic agent.
   260. The modified cell or the method of any one of embodiments 256-259, wherein expression and/or secretion of the therapeutic agent is regulated by an inducible expression system and/or the modified cell comprises a polynucleotide encoding an inducible suicide system.
   261. The modified cell or the method of any one of embodiments 256-259, wherein a range of concentration values of IL6 is 60 to 5000 μg/ml, 200-5000 μg/ml, or 2000-5000 μg/ml in the blood of the subject.
   262. The modified cell or the method of any one of embodiments 256-259, wherein a range of concentration values IFN-γ is 20 to 5000 μg/ml, 200 to 5000 μg/ml, or 500 to 5000 μg/ml in the blood of the subject.
   263. The modified cell or the method of any one of embodiments 256-262, wherein the administering the effective amount of the therapeutic agent comprises intravenous delivery of an amount of human IL-6 in the range of about 0.5-50 ug per kilogram of body weight.
   264. The modified cell or the method of any one of embodiments 256-263, wherein the modified cell or the T cells comprise an additional CAR binding a solid tumor antigen, and the (first) CAR binds an antigen of a white blood cell.
   265. The modified cell or the method of embodiment 264, wherein the solid tumor antigen is tMUC 1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and the white blood cells is a B cell antigen and the B cell antigen is CD19, CD20, CD22, or BCMA.
   266. The modified cell or the method of any one of embodiments 256-265, wherein the modified cell or the T cells comprise a dominant negative form of PD-1.
   267. The modified cell or the method of any one of embodiments 259-266, wherein the modified cell or the T cells comprise a modified PD-1 lacking a functional PD-1 intracellular domain.
   268. The isolated polynucleotide, the population of CAR cells, the pharmaceutical composition, modified cell, or the method of any one of embodiments 246-267, wherein the CAR comprises an extracellular domain, a transmembrane domain, and an intracellular domain, the extracellular domain binds an antigen.
   269. The isolated polynucleotide, the population of CAR cells, the pharmaceutical composition, modified cell, or the method of any one of embodiments 246-268, wherein the intracellular domain comprises a co-stimulatory domain that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and one combination thereof.
   270. The isolated polynucleotide, the population of CAR cells, the pharmaceutical composition, modified cell, or the method of any one of embodiments 246-269, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Rα2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
   271. The isolated polynucleotide, the population of CAR cells, the pharmaceutical composition, modified cell, or the method of any one of embodiments 246-270, wherein the therapeutic agent is present in the modified cell in a recombinant DNA construct, in an mRNA, or in a viral vector.
   272. The isolated polynucleotide, the population of CAR cells, the pharmaceutical composition, modified cell, or the method of any one of embodiments 246-271, wherein the modified cell comprises a therapeutic agent mRNA encoding the therapeutic agent, and the mRNA is not integrated into the genome of the modified cell.
   273. The isolated polynucleotide, the population of CAR cells, the pharmaceutical composition, modified cell, or the method of any one of embodiments 246-272, wherein the modified cell comprises a polynucleotide comprising a promoter comprising a binding site for a transcription modulator that modulates the expression and/or secretion of the therapeutic agent in the cell.
   274. The isolated polynucleotide, the population of CAR cells, the pharmaceutical composition, modified cell, or the method of embodiment 273, wherein the transcription modulator is or includes Hif1a, NFAT, FOXP3, and/or NFkB.
   275. The isolated polynucleotide, the population of CAR cells, the pharmaceutical composition, modified cell, or the method of embodiment 273, wherein the promoter is responsive to the transcription modulator.
   276. The isolated polynucleotide, the population of CAR cells, the pharmaceutical composition, modified cell, or the method of embodiment 273, wherein the promoter is operably linked to the polynucleotide encoding the therapeutic agent such that the promoter drives expression and/or secretion of the therapeutic agent in the cell.
   277. The isolated polynucleotide, the population of CAR cells, the pharmaceutical composition, modified cell, or the method of any one of embodiments 273-276, wherein the promoter comprises at least one of SEQ ID NOs: 323-325.
   278. A method of using any one of the preceding embodiments (1-277) in autologous T cell therapy, allogenic T cell therapy, TCR T cell therapy, or NK cell therapy.
   279. The CAR described in any one of the preceding embodiments (1-278), wherein the CAR comprises one or more of the complementarity determining regions (CDRs) that bind an antigen of interest.
   280. A polynucleotide encoding one or more therapeutic agents comprising at least two cytokines.
   281. A modified cell comprising a polynucleotide encoding one or more therapeutic agents.
   282. A method for enhancing T cell response (e.g., expansion and/or activation) invitro and/or in vivo, the method comprising: introducing a polynucleotide encoding one or more therapeutic agents to obtain a modified cell; culturing the modified cell to obtain a population of modified cells; and contacting the population of modified cells with cells including an antigen, wherein the modified cells enhance the T cell response as compared to the cell contacted with modified cells not comprising the polynucleotide.
   283. The polynucleotide, modified cell, or method of any one of embodiments 280-282, wherein the one or more therapeutic agents comprise at least two of 1L6, 1L12, or IFNγ.
   284. The polynucleotide, modified cell, or method of any one of embodiments 280-282, wherein the one or more therapeutic agents comprise at least IL12 and IFNγ.
   285. The polynucleotide, modified cell, or method of any one of embodiments 280-282, wherein the one or more therapeutic agents comprise at least two of IL12, IL6, IFNγ, IFNβ, TNFα, or Neo-2/15.
   286. The modified cell of embodiment 281, wherein the modified cell comprises a polynucleotide encoding IL12 and IFNγ.
   287. The modified cell of embodiment 281, wherein the modified cell comprises a polynucleotide encoding TNFα.
   288. The modified cell of embodiment 287, wherein the polynucleotide encoding TNFα is linked to a HIF VHL binding domain.
   289. The polynucleotide, modified cell, or method of any one of embodiments 283-288, wherein the one or more therapeutic agents comprise at least one cytokine associated with an oxygen-sensitive polypeptide domain.
   290. The polynucleotide, modified cell, or method of embodiment 289, wherein the oxygen-sensitive polypeptide domain comprises a HIF VHL binding domain.
   291. The polynucleotide, modified cell, or method of embodiment 289, wherein the oxygen-sensitive polypeptide domain is or comprises SEQ ID NO: 457.
   292. The polynucleotide, modified cell, or method of embodiment 289, wherein the polynucleotide encodes a cytokine, an EA linker, and SEQ ID NO: 457.
   293. The polynucleotide, modified cell, or method of embodiment 289, wherein the polynucleotide encodes or the modified cell comprises at least one of SEQ ID NO: 470-489 or 491-495.
   294. The polynucleotide, modified cell, or method of any one of embodiments 280-293, wherein the polynucleotide comprises a promoter comprising a binding site for a transcription modulator that modulates the expression and/or secretion of the therapeutic agent in the cell.
   295. The polynucleotide, modified cell, or method of embodiment 294, wherein the transcription modulator is or includes Hif1a, NFAT, FOXP3, and/or NFkB.
   296. The polynucleotide, modified cell, or method of any one of embodiments 280-295, wherein the polynucleotide encodes a binding molecule.
   297. The modified cell or method of any one of embodiments 280-296, wherein the binding molecule is CAR or TCR.
   298. The polynucleotide, modified cell, or method of embodiment 297, wherein the polynucleotide is present in the modified cell in a recombinant DNA construct, in an mRNA, or in a viral vector.
   299. The polynucleotide, modified cell, or method of embodiment 298, wherein the polynucleotide is an mRNA, which is not integrated into the genome of the modified cell.
   300. The polynucleotide, modified cell, or method of any one of embodiments 280-299, wherein the polynucleotide comprises a sequence encoding a cleavable peptide (e.g., 2A peptide or IRES), which is disposed between the at least two cytokines.
   301. A composition for treating a subject having cancer, the composition comprising a first population of cells and a second population of cells, wherein the first population of cells comprising a polynucleotide encoding a binding molecule binding a solid tumor antigen, a second population of cells comprising the polynucleotide of any one of embodiments 280-300.
   302. The composition of embodiment 301, wherein the polynucleotide comprises a sequence encoding CAR binding CD19, a sequence encoding IL6, and a sequence encoding IFNγ.
   303. The composition of embodiment 301, wherein the polynucleotide comprises a sequence encoding CAR binding CD19, a sequence encoding IL12, and a sequence encoding IFNγ.
   304. The composition of embodiment 301, wherein the binding molecule is a CAR or a TCR.
   305. The composition of embodiment 301, wherein the first population of cells comprises a polynucleotide encoding TNFα that is linked to a HIF VHL binding domain.
   306. A composition for treating a subject having cancer, the composition comprising a first population of cells and a second population of cells, wherein the first population of cells comprising a polynucleotide encoding a binding molecule binding a white blood antigen, a second population of cells comprising the polynucleotide of any one of embodiments 280-300.
   307. The composition of embodiment 306, wherein the polynucleotide comprises a sequence encoding a binding molecule binding a solid tumor antigen, a sequence encoding 1L6, and a sequence encoding IFNγ.
   308. The composition of embodiment 306, wherein the polynucleotide comprises a sequence encoding a binding molecule binding a solid tumor antigen, a sequence encoding 1L12, and a sequence encoding IFNγ.
   309. The composition of any one of embodiments 306-308, wherein the binding molecule is a CAR or a TCR.
   310. The composition of embodiment 306, wherein the first population of cells comprise a polynucleotide encoding TNFα that is linked to a HIF VHL binding domain.
   311. A method of causing or inducing a T cell response, enhancing the T cell response, treating a subject having cancer, or enhancing the treatment, the method comprising: administering an effective amount of the composition of any one of embodiments 301-310 to a subject having cancer.
   312. A cell modified to express one or more molecules at a level that is higher than the level of the one or more molecules expressed by a cell that has not been modified to express the one or more molecules, wherein the one or more molecules comprise a cytokine (for example, IFN-γ, IL-12, and IL-6) or a derivatives thereof.
   313. A modified cell comprising an antigen binding molecule and one or more molecules, wherein expression and/or function of one or more molecules in the modified cell has been enhanced, wherein the one or more molecules comprise a cytokine (for example, IFN-γ, IL-12, and IL-6).
   314. The modified cell of any one of embodiments 312 and 313, wherein the modified cell comprises a disruption in an endogenous gene or an addition of an exogenous gene that is associated with a biosynthesis or transportation pathway of the one or more molecules.
   315. A method or use of polynucleotide, the method comprising providing a viral particle (e.g., AAV, lentivirus or their variants) comprising a vector genome, the vector genome comprising the polynucleotide encoding one more cytokines and a polynucleotide encoding a binding molecule, the polynucleotide operably linked to an expression control element conferring transcription of the polynucleotides; and administering an amount of the viral particle to a subject such that the polynucleotide is expressed in the subject. Examples of cytokines include IFN-γ, IL-12, and IL-6.
   316. The method of embodiment 315, wherein the AAV preparation may include AAV vector particles, empty capsids and host cell impurities, thereby a 2A peptide providing an AAV product substantially free of AAV empty capsids.
   317. A pharmaceutical composition comprising the population of the cells of any one of embodiments 312-314.
   318. A method of causing or eliciting T cell response in a subject in need thereof and/or treating a tumor of the subject, the method comprising administering an effective amount of the composition of embodiment 317 to the subject.
   319. A method of enhancing a cell therapy, the method comprising administering an effective amount of the composition of embodiment 317 to the subject, wherein a number of M2 macrophages in the subject has been reduced as compared to the M2 macrophages in the subject administered with an effective amount of the modified cells of which expression of IFN-γ has not been enhanced, wherein the cytokine is IFNγ.
   320. A method of reducing a number of M2 macrophages, the method comprising contacting M2 macrophages with an effective amount of the composition of embodiment 317 to the subject, wherein a number of M2 macrophages in the subject has been reduced as compared to M2 macrophages in the subject administered with an effective amount of the modified cells of which expression of IFN-γ has not been enhanced, wherein the cytokine is IFNγ.
   321. The method of any one of embodiments 319 and 320, wherein the expression of IFN-γ is regulated such that the modified cells express a higher level of IFN-γ in response to activation of the modified cells.
   322. An isolated polynucleotide encoding one or more molecules comprising IFN-γ.
   323. The isolated polynucleotide, modified cell, method, or pharmaceutical composition of any one of embodiments 312-322, wherein the one or more molecules further comprise IL-16.
   324. The modified cell of any one of embodiments 312-323, wherein the modified cell comprises the antigen binding molecule, wherein the antigen binding molecule is a chimeric antigen receptor (CAR), which comprises an antigen-binding domain, a transmembrane domain, and an intracellular signaling domain.
   325. The modified cell of embodiment 324, wherein the antigen-binding domain binds a tumor antigen selected from a group consisting of TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Rα2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
   326. The modified cell of any one of embodiments 324 and 325, wherein the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1 BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
   327. The modified cell of any one of embodiments 312-318 and 319-323, wherein the modified cell comprises the antigen binding molecule, the antigen binding molecule is a modified TCR.
   328. The modified cell of embodiment 327, wherein the TCR is derived from spontaneously occurring tumor-specific T cells in patients.
   329. The modified cell of embodiment 328, wherein the TCR binds a tumor antigen.
   330. The modified cell of embodiment 329, wherein the tumor antigen comprises CEA, gp100, MART-1, p53, MAGE-A3, or NY-ESO-1.
   331. The modified cell of embodiment 329, wherein the TCR comprises TCRγ and TCRδ Chains or TCRα and TCRβ chains, or a combination thereof.
   332. The modified cell of any of the preceding embodiments (312-331), wherein the cell is an immune effector cell (e.g., a population of immune effector cells).
   333. The modified cell of embodiment 332, wherein the immune effector cell is a T cell or an NK cell.
   334. The modified cell of embodiment 333, wherein the immune effector cell is a T cell.
   335. The modified cell of embodiment 333 wherein the T cell is a CD4+ T cell, a CD8+ T cell, or a combination thereof.
   336. The modified cell of any one of embodiments 312-336, wherein the cell is a human cell.
   337. The modified cell of any one of the preceding embodiments 312-336, wherein the enhanced expression and/or function of the one or more molecules is implemented by introducing a polynucleotide encoding the one or more molecules and/or the binding molecule, which is present in the modified cell in a recombinant DNA construct, in an mRNA, or in a viral vector.
   338. The modified cell of embodiment 337, wherein the polynucleotide is an mRNA, which is not integrated into the genome of the modified cell.
   339. The modified cell of embodiment 337, wherein the polynucleotide is associated with an oxygen-sensitive polypeptide domain.
   340. The modified cell of embodiment 339, wherein the oxygen-sensitive polypeptide domain comprises HIF VHL binding domain.
   341. The modified cell of embodiment 337, wherein the polynucleotide is regulated by a promoter comprising a binding site for a transcription modulator that modulates the expression and/or secretion of the therapeutic agent in the cell.
   342. The modified cell of embodiment 341, wherein the transcription modulator is or includes Hif1a, NFAT, FOXP3, and/or NFkB.
   343. A pharmaceutical composition comprising a population of the modified cells of any one of embodiments 311-342, wherein the cells are T cells.
   344. The pharmaceutical composition of embodiment 343, wherein the promoter is responsive to the transcription modulator.
   335. The pharmaceutical composition of embodiment 323, wherein the promoter is operably linked to the polynucleotide encoding the one or more molecules, and wherein the promoter drives expression and/or secretion of the one or more molecules in the cell.
   346. The pharmaceutical composition of embodiment 343, wherein the promoter comprises at least a sequence of SEQ ID NOs: 323, 324, or 325.
   347. The pharmaceutical composition of embodiment 343, wherein the modified T cell comprises polynucleotides encoding SEQ ID NOs: 286 and 469, and a polynucleotide encoding SEQ ID NO: 328.
   348. The pharmaceutical composition of embodiment 343, wherein the CAR and the one or more molecules are produced in the form of a polyprotein, which is cleaved to generate separate CAR and the one or more therapeutic agents, and wherein a cleavable moiety is between the CAR and the one or more therapeutic agents, the cleavable moiety comprising a 2A peptide, and the 2A peptide comprising P2A or T2A.
   349. The pharmaceutical composition of embodiment 343, wherein the modified T cell comprises an additional CAR, the CAR binds a white blood cell (e.g., CD19), and the additional CAR binds solid tumor antigen.
   350. The pharmaceutical composition of embodiment 343, wherein the modified T cell comprises an additional CAR, the CAR binds a solid tumor antigen, and the additional CAR binds an antigen of a white blood cell.
   351. The pharmaceutical composition of embodiment 349, wherein the solid tumor antigen is tMUC 1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, PSCA, or EGFR, and wherein the antigen of the white blood cell is CD19, CD20, CD22, or BCMA.
   352. The pharmaceutical composition of embodiment 349, wherein the modified T cell comprises a dominant negative form of PD-1.
   353. The modified cell of any one of embodiments 311-352, wherein the modified cell is capable of reducing M2 macrophages and/or converting M2 macrophages to M1 macrophages.
   354. A modified cell comprising an antigen binding molecule, wherein expression and/or function of one or more molecules in the modified cell has been increased or enhanced, the one or more molecules comprising a cytokine.
   355. A modified cell comprising an antigen binding molecule, wherein expression and/or function of one or more molecules in the modified cell has been increased or enhanced, the one or more molecules comprising at least one of IL-6, IL-12, IL-7, IL-15, and IFNγ.
   356. A modified cell comprising an antigen binding molecule, wherein expression and/or function of one or more molecules in the modified cell has been increased or enhanced, the one or more molecules comprising IL-6 and IFNγ.
   357. A modified cell comprising an antigen binding molecule, wherein expression and/or function of one or more molecules in the modified cell has been increased or enhanced, the one or more molecules comprising IL-12.
   358. A plurality of modified cells comprising an antigen binding molecule, wherein expression and/or function of one or more molecules in the plurality of modified cells modified cell has been increased or enhanced, the one or more molecules comprising at least one of IL-6, IL-12, and IFNγ.
   359. The plurality of modified cells of embodiment 358, wherein the plurality of modified cells comprise the modified cell of embodiment 357 and the modified cell of embodiment 358.
   360. A pharmaceutical composition comprising the population of the modified cell(s) of any one of embodiments 354-359.
   361. A method of enhancing, causing or eliciting T cell response in a subject in need thereof, enhancing T cell expansion, enhancing treatment or inhibition of tumor growth, and/or treating a tumor of the subject, the method comprising administering an effective amount of the composition of embodiment 360 to the subject, the expansion of the modified cells(s), T cell response caused by the modified cell(s), or inhibition of tumor growth enhanced as compared to the modified cell of which expression and/or function of the one or more molecules has not been enhanced.
   362. The modified cell of any preceding embodiments 354-361, wherein the one or more molecules comprise a derivative of the one or more molecules.
   363. The modified cell of any one of embodiments 354-362, wherein the modified cell comprises a polynucleotide encoding one or more components of a gene editing system associated with the one or more genes.
   364. The modified cell of any one of embodiments 354-362, wherein the enhanced expression and/or function of the one or more genes is implemented by introducing a polynucleotide of the one or more genes encoding the one or more molecules, which is present in the modified cell in a recombinant DNA construct, in an mRNA, or in a viral vector.
   365. The modified cell of embodiments 363 or 364, wherein the polynucleotide is an mRNA, which is not integrated into the genome of the modified cell.
   366. The modified cell of any one of embodiments 354-362, wherein the modified cell comprises one or more polynucleotides encoding the one or more molecules, and the one or more polynucleotides are associated with an oxygen-sensitive polypeptide domain.
   367. The modified cell of embodiment 366, wherein the oxygen-sensitive polypeptide domain comprises a HIF VHL binding domain.
   368. The modified cell of embodiments 366 or 367, wherein the one or more polynucleotides are regulated by a promoter comprising a binding site for a transcription modulator that modulates the expression and/or secretion of the therapeutic agent in the cell.
   369. The modified cell of embodiment 368, wherein the transcription modulator is or includes Hif1a, NFAT, FOXP3, and/or NFkB.
   370. The modified cell of any one of embodiments 356-361, wherein the modified cell comprises a nucleotide sequence encoding a nuclear factor of activated T-cells (NFAT) promoter operatively associated with a polynucleotide encoding IL-6 and IFNγ.
   371. The modified cell of any one of embodiments 356-361, wherein the modified cell comprises a nucleotide sequence encoding a nuclear factor of activated T-cells (NFAT) promoter operatively associated with a polynucleotide encoding IL-6, 2A peptide, and IFNγ.
   372. The modified cell of any one of embodiments 356-361, 370, and 371, wherein the modified cell comprises a nucleotide sequence encoding a nuclear factor of activated T-cells (NFAT) promoter operatively associated with a polynucleotide encoding IL-12 associated with a HIF VHL binding domain.
   373. The modified cell of any one of embodiments 356-361, 370, and 371, wherein the modified cell comprises a nucleotide sequence encoding a nuclear factor of activated T-cells (NFAT) promoter operatively associated with a polynucleotide encoding IL-12. 374. The modified cell of any one of embodiments 370-373, wherein the NFAT promoter is located 3′ of the nucleotide sequence encoding the one or more molecules.
   375. The modified cell of any one of embodiments 354-374, wherein the one or more molecules are one or more human cytokines.
   376. The modified cell of any one of embodiments 354-375, wherein the modified cell comprises a sequence of at least one of SEQ ID Nos: 328 or 456.
   377. The modified cell of any one of embodiments 354-375, wherein the modified cell comprises a sequence of at least one of SEQ ID NOs: 511-525.
   378. The modified cell of any one of embodiments 354-375, wherein the modified cell comprises a sequence of at least one of SEQ ID NOs: 328, 456, 470, 471, 474, 475, 478-480, 482, 483, 488, 489, 483, 494, or 511-525.
   379. The modified cell of any one of embodiments 354-375, wherein the modified cell comprises a sequence of at least one of SEQ ID NOs: 278, 327, or 328 in the order of N-terminus to C-terminus.
   380. The modified cell of any one of embodiments 354-375, wherein the modified cell comprises a sequence of at least one of SEQ ID NOs: 478 or 479.
   381. The modified cell of any one of embodiments 354-375, wherein the modified cell comprises a sequence of at least one of SEQ ID NOs:450, 470-473, 480, 481, 486, or 487.
   382. The modified cell of any one of embodiments 354-375, wherein the modified cell comprises a sequence of at least one of SEQ ID NOs: 328, 287, or 450.
   383. The plurality of modified cells of embodiment 358, wherein the plurality of modified cells comprises at least one of the modified cells of embodiments 370-383.
   384. A polynucleotide comprising the nucleotide sequence(s) of any one of embodiments 363-383 and/or a nucleotide sequence encoding the binding molecule.
   385. The modified cell of any one of embodiments 354-374, wherein the modified cell comprises an addition of exogenous gene that are associated with a biosynthesis or transportation pathway of the one or more molecules.
   386. The modified cell of any one of embodiments 354-385, wherein the antigen binding molecule is chimeric antigen receptor (CAR), which comprises an antigen-binding domain, a transmembrane domain, and an intracellular signaling domain.
   387. The modified cell of embodiment 386, wherein the antigen-binding domain binds a tumor antigen selected from the group consisting of TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Rα2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
   388. The modified cell of any one of embodiments 386 and 387, wherein the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1 BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
   389. The modified cell of any one of embodiments 354-384, wherein the antigen binding molecule is a modified TCR.
   390. The modified cell of embodiment 389, wherein the TCR is derived from spontaneously occurring tumor-specific T cells in patients.
   391. The modified cell of embodiment 390 wherein the TCR binds a tumor antigen.
   392. The modified cell of embodiment 391, wherein the tumor antigen comprises CEA, gp100, MART-1, p53, MAGE-A3, or NY-ESO-1.
   393. The modified cell of embodiment 391, wherein the TCR comprises TCRγ and TCRδ chains or TCRα and TCRβ chains, or a combination thereof.
   394. A modified cell comprising an antigen binding molecule, wherein expression and/or function of IL-12 in the modified cell has been increased or enhanced.
   395. A modified cell comprising an antigen binding molecule, wherein expression and/or function of IL-6 in the modified cell has been increased or enhanced.
   396. A modified cell comprising an antigen binding molecule, wherein expression and/or function of IL-6 and IFNγ in the modified cell has been increased or enhanced.
   397. A modified cell comprising an antigen binding molecule, wherein expression and/or function of IFNγ in the modified cell has been increased or enhanced.
   398. A plurality of modified cells comprising the modified cells of embodiments 394 and 396.
   399. A plurality of modified cells comprising at least one of the modified cells of embodiments 394, 395, 396, or 397.
   400. The plurality of modified cells of embodiments 398 or 399, wherein the antigen binding molecule is a CAR binding to an antigen of WBC (e.g., CD19).
   401. The plurality of modified cells of any one of embodiments 398 and 399, wherein the antigen binding molecule is a CAR binding to a solid tumor antigen (e.g., tMUC1).
   402. The plurality of modified cells of any one of embodiments 398 and 399, wherein the plurality of modified cells comprise a first modified cell comprising a polynucleotide encoding IL-12 and/or a second modified cell comprising a polynucleotide encoding IL-6 and IFNγ, the antigen binding molecule is a CAR binding to an antigen of WBC (e.g., CD19), and the expression of IL-12, IL-6, and IFNγ is regulated by NFAT promoter.
   403. The modified cell of any preceding embodiments 394-401, wherein the modified cell comprises a polynucleotide encoding IL-12, IL-6 and IFNγ, IL-16, or IFNγ.
   404. The modified cell of embodiment 403, wherein the polynucleotide is operatively linked to a polynucleotide encoding NFAT.
   405. The modified cell of embodiment 404, wherein expression of IL-12, IL-6 and IFNγ, IL-16, or IFNγ is enhanced in the modified cell when the modified cell is activated.
   406. The modified cell of embodiment 404, wherein the polynucleotide encoding IL-12 is associated with a HIF VHL binding domain such that maintenance and/or function of the expressed IL-12 is oxygen-sensitive.
   407. The plurality of modified cells of embodiment 400, wherein the plurality of modified cells further comprise an additional modified cell comprising an additional binding molecule (e.g., CAR or TCR) binding a solid tumor antigen.
   408. The plurality of modified cells of embodiment 407, wherein the additional modified cell is an additional CAR binding a solid tumor antigen.
   409. The modified cell of any one of embodiments 394-408, wherein the modified cell is a human T cell derived form a subject or a healthy donor.
   410. The modified cell of any one of embodiments 394-409, wherein the cell is an immune effector cell.
   411. The modified cell of embodiment 410, wherein the immune effector cell is a T cell or an NK cell.
   412. The modified cell of embodiment 411, wherein the immune effector cell is a T cell.
   413. The modified cell of embodiment 412, wherein the T cell is a CD4+ T cell, a CD8+ T cell, or a combination thereof.
   414. The modified cell of any one of embodiments 354-412, wherein the cell is a human cell.
   415. The modified cell of any one of embodiments 354-412, wherein the modified cell comprises more than one binding molecule.
   416. A plurality of modified cells of any one of embodiments 354-415, wherein the plurality of modified cells comprise a modified cell comprises a CAR binding an antigen of WBC (e.g., CD19) and a CAR binding a solid tumor antigen (e.g., tMUC1).
   417. The modified cell of any one of embodiments 354-416, wherein the modified cell comprises a dominant negative form of PD1.
   418. A polynucleotide comprising a polynucleotide encoding a promoter operatively linked to a polynucleotide encoding a therapeutic agent and a polynucleotide encoding a CAR binding a cell surface molecule of WBC. An example of the polynucleotide is provided in FIG. 56.
   419. The polynucleotide of embodiment 418, wherein the promoter comprises at least one of Hif1a, NFAT, FOXP3, NFKB, AP-1, AT-hook, and/or NFkB.
   420. The polynucleotide of embodiment 418, wherein the promoter is NFAT such that a cell comprising the polynucleotide express the therapeutic agent in response to activation of the cell.
   421. The polynucleotide of any one of embodiments 418-420, wherein the cell surface molecule is CD19, CD22, CD20, BCMA, CD5, CD7, CD2, CD16, CD56, CD30, CD14, CD68, CD11b, CD18, CD169, CD1c, CD33, CD38, CD138, or CD13.
   422. The polynucleotide of any one of embodiments 418-420, wherein the cell surface molecule is CD19, CD20, CD22, or BCMA.
   423. The polynucleotide of any one of embodiments 418-420, wherein the cell surface molecule is CD19 or BCMA.
   424. The polynucleotide any one of embodiments 418-423, wherein the therapeutic agent comprises a cytokine.
   425. The polynucleotide of any one of embodiments 418-423, wherein the therapeutic agent comprises at least one of IFN-γ, IL-2, IL-6, IL-7, IL-15, IL-17, or IL-23.
   426. The polynucleotide of any one of embodiments 418-423, wherein the therapeutic agent comprises IL-12.
   427. The polynucleotide of any one of embodiments 418-423, wherein the therapeutic agent comprises IL-6, IFNγ, or a combination thereof.
   428. The polynucleotide of any one of embodiments 418-427, wherein the polynucleotide encoding the therapeutic agent further encodes a HIF VHL binding domain linked to the therapeutic agent such that expression of the therapeutic agent by a cell comprising the polynucleotide is regulated by a level of oxygen.
   429. A composition comprising a plurality of polynucleotides comprising the polynucleotide of any one of embodiments 418-428 and an additional polynucleotide encoding a CAR binding a solid tumor antigen.
   430. A composition comprising a plurality of polynucleotides comprising two polynucleotides of any one of embodiments 418-428 and an additional polynucleotide encoding a CAR binding a solid tumor antigen, wherein the therapeutic agents of two polynucleotides are different.
   431. The composition of any one of embodiments 429 and 430, wherein the additional polynucleotide encoding a CAR binding a solid tumor antigen further comprises a polynucleotide encoding a molecule or the therapeutic agent of any one of embodiments 418-428.
   432. The composition of embodiment 431, wherein the molecule is a modified checkpoint protein (e.g., dominant negative forms of PD1 or PDL-1).
   433. A modified cell comprising the polynucleotide of any one of embodiments 418-428 and/or the composition of any one of embodiments 429-432.
   434. A method of causing, inducing, or enhancing T cell response of a subject having a tumor or treating the subject, the method comprising administering an effective amount of a pharmaceutical composition comprising the modified cells of embodiment 433.
   434. The method of embodiment of 434, wherein the tumor is a solid tumor.
   435. A modified cell comprising the polynucleotide of any one of embodiments 418-428.
   436. A method of causing, inducing, or enhancing T cell response of a subject having leukemia, lymphoma, or multiple myeloma or treating the subject, the method comprising administering an effective amount of a pharmaceutical composition comprising modified cells of embodiment 435.
   437. The method of embodiment 436, wherein the CAR binds CD19, the subject has leukemia or lymphoma, and the therapeutic agent comprises IL-6 and IFNγ.
   438. The method of embodiment 437, wherein the modified cell further comprises a polynucleotide encoding IL-12.
   439. The method of embodiment 436, wherein the CAR binds BCMA, the subject has myeloma, and the therapeutic agent comprises IL-6 and IFNγ.
   440. The method of embodiment 439, wherein the modified cell further comprises a polynucleotide encoding IL-12.
   441. A method for treating a subject having cancer, the method comprising:
contacting T cells with a plurality of polynucleotides comprising a first polynucleotide encoding a CAR binding a B cell antigen and one or more cytokines and a second polynucleotide encoding a CAR binding a solid tumor antigen to obtain modified T cells;
administering an effective amount of the modified T cells to the subject.
   442. A method for enhancing treatment of a subject having cancer, the method comprising: contacting T cells with a plurality of polynucleotides comprising a first polynucleotide encoding a CAR binding a B cell antigen and one or more cytokines and a second polynucleotide encoding a CAR binding a solid tumor antigen to obtain modified T cells;
administering an effective amount of the modified cells to the subject, wherein the modified T cells enhance T cell response to the CAR T cell infusion, the T cell response comprising at least one of T cell expansion, T cell activation, cytokine releases, and inhibition on tumor growth in the subject as compared to a subject administered with modified T cells comprising the CAR binding the solid tumor antigen or administered with modified T cells comprising the CAR binding the B cell antigen, or a combination thereof.
   443. The method of embodiment 441 or 442, further comprising culturing the modified T cells; and 444. The method of embodiment 441 or 442, wherein the one or more cytokines comprise IL-6 and/or IFNγ.
   445. The method of embodiment 441 or 442, wherein expression and/or function of one or more proteins in at least one portion of the modified T cell has been increased or enhanced, and the one or more proteins comprise IL-6 or its derivatives, IFNγ or its derivatives, or a combination thereof.
   446. The method of embodiment 444, wherein at least one portion of the modified T cells express or secrete the one or more proteins in response to activation of at least one portion of the modified T cells or hypoxia, or a combination thereof.
   447. The method of embodiment 444, wherein IL-6 is human IL-6, and IFNγ is human IFNγ.
   448. The method of embodiment 444, wherein the modified T cells comprise an exogenous polynucleotide encoding the one or more proteins.
   449. The method of embodiment 448, wherein the exogenous polynucleotide is present in the modified T cell in a recombinant DNA construct, in an mRNA, or in a viral vector.
   450. The method of embodiment 448, wherein the exogenous polynucleotide comprises a polynucleotide encoding SEQ ID NOs: 287 and a polynucleotide encoding SEQ ID NO: 328.
   451. The method of embodiment 448, wherein the exogenous polynucleotide comprises a promoter comprising a binding site for a transcription modulator that modulates the expression and/or secretion of the one or more proteins in the modified cell.
   452. The method of embodiment 451, wherein the transcription modulator is or comprises Hif1a, NFAT, FOXP3, or NFkB.
   453. The method of embodiment 444, wherein the antigen binding molecule and the one or more proteins are produced in the form of a polyprotein, which is cleaved to generate separate antigen binding molecule and the one or more therapeutic agent molecules, and wherein a cleavable moiety is between the antigen binding molecule and the one or more proteins.
   454. The method of embodiment 444, wherein the plurality of polynucleotides comprising a third polynucleotide encoding a CAR binding a B cell antigen and IL-12 or its derivatives.
   455. The method of embodiment 454, wherein the modified T cells express and secrete human IL-12 in response to activation of the modified T cells or hypoxia, or a combination thereof.
   456. The method of any of embodiments 441-455, wherein the CAR comprises an extracellular domain, a transmembrane domain, and an intracellular domain, the extracellular domain binding an antigen.
   457. The method of embodiment 456, wherein the CAR binds TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Rα2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
   458. The method of embodiment 457, wherein the intracellular domain comprises a co-stimulatory domain that comprises an intracellular domain of a co-stimulatory molecule selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D.
   459. The method of embodiment 456, wherein the CAR binds the B cell antigen selected from the group consisting of CD19, CD22, CD20, BCMA, CD5, CD7, CD2, CD16, CD56, CD30, CD14, CD68, CD11b, CD18, CD169, CD1c, CD33, CD38, CD138, and CD13.
   460. The method of any of embodiments 441-459, wherein the cancer is thyroid cancer, the B cell antigen is CD19, and the solid tumor antigen is TSHR.
   461. The method of any of embodiments 441-459, wherein the cancer is colorectal cancer, the B cell antigen is CD19, and the solid tumor antigen is GUCY2C.
   462. A pharmaceutical composition comprising a first population of CAR T cells binding TSHR or GUCY2C, a second population of CAR T cells binding CD19, wherein expression of one or more cytokines of the second population of CAR cells has been enhanced, the one or more cytokines comprising at least one of IL-6, IL-12, and IFNγ.
   463. The pharmaceutical composition of embodiment 462, wherein the pharmaceutical composition further comprises a third population of CAR T cells binding CD10 and TSHR or GUCY2C.
   464. A pharmaceutical composition comprising the modified cells of any of embodiments 441-461.
   465. A method of enhancing cytokine release, the method comprising:
introducing a polynucleotide encoding one or more cytokines into cells to obtain modified cells or introducing a molecule that enhances expression and/or functions of the one or more cytokines into cells to obtain modified cells; contacting the modified cells with cells comprising an antigen (e.g., solid tumor antigen); and
measuring a level of the cytokine release by the modified cells, wherein the level of the cytokine released in response to the cells expressing the antigen is higher than the level of the cytokine released by cells that were not introduced with the polynucleotide or the molecule and contacted with cells expressing the antigen.
   466. The method of embodiment 465, wherein the one or more cytokines comprise IL-6 and/or IFNγ, the modified cells overexpress IL-6 and/or IFNγ in response to the cells expressing the antigen, and the level of the cytokine released by the modified cells in response to the cells is higher than the level of the cytokine released by cells that don't overexpress IL-6 and/or IFNγ.
   467. The method of embodiment 466, wherein the modified cells further overexpress IL-12, the level of the cytokine released by the modified cells in response to the cells expressing the antigen is higher than the level of the cytokine released by cells that overexpress IL-6 and/or IFNγ but not IL-12.
   468. The method of embodiment 467, wherein the cytokine release comprises a cytokine release of at least one of IL6, IFNγ, TNF-α, GZMB.
   468. A method of enhancing T cell response, the method comprising: introducing a polynucleotide encoding IFNγ into T cells; culturing the T cells; contacting the T cells with a cell expressing an antigen; and measuring a level of T cell response of the T cells in response to the cell expressing the antigen, wherein the level of T cell response of the T cells is higher than the level of T cell response of T cells without the polynucleotide encoding IFNγ.
   469. The method of embodiment 468, further comprising: introducing a polynucleotide encoding IL-6 into T cells, wherein the level of T cell response of the T cells is higher than the level of T cell response of T cells without the polynucleotide encoding IL-6 but having the polynucleotide encoding IFNγ.
   470. The method of embodiment 468 or 469, wherein the T cell response comprises a cytokine release, which includes the release of at least one of IL-2, IL-6, TNFα, or IFNγ.
   471. A method of enhancing T cell response, the method comprising: introducing a polynucleotide encoding IL-6 into T cells; culturing the T cells; contacting the T cells with a cell expressing an antigen; and measuring a level of T cell response of the T cells in response to the cell expressing the antigen, wherein the level of T cell response of the T cells is higher than the level of T cell response of T cells without the polynucleotide encoding IL-6.
   472. The method of embodiment 471, further comprising: introducing a polynucleotide encoding IL-12 into T cells, wherein the level of T cell response of the T cells is higher than the level of T cell response of T cells without the polynucleotide encoding IL-12 but having the polynucleotide encoding IL-6.
   473. The method of any embodiments 471 or 472, wherein the T cell response comprises a cytokine release, which includes at least one of IL-12, TNFα, and IFNγ.
   474. The method of any one of embodiments 465-473, wherein the modified cells orT cells comprise the binding molecule of any one of embodiments 465-473.
   475. The method of any of embodiments 465-473, wherein the antigen is the solid tumor antigen or the WBC antigen.
   476. The method of any one of embodiments 465-468, 474, or 475, wherein the cells are T cells, NK cells, DCs, and/or macrophages.
   477. The method of any one of embodiments 465-475, wherein the cells or the T cells are human cells.
   478. The method of any one of embodiments 465-468, 474, or 475, wherein the cells are human T cells.
   479. A composition comprising one or more vectors described in Embodiment 1 and Embodiment 2 of FIG. 56 and/or in Table 11. In embodiments, the one or more vectors are Vector 1, Vector 2, Vector 3, or Vector 4 of Table 11.
   480. A method of preparing CAR T cells, the method comprising: obtaining T cells from healthy donor or a subject; contacting the T cells with the composition of embodiment 479 to obtain the CAR T cells; culturing the CAR T cells.
   481. The method of embodiment 480, further comprising: determining a multiplicity of infection value associated with the contacting such that a number of T cells expressing a single type of CAR molecule (e.g., CD19 CAR or TSHR CAR) is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) than T cells expressing more than one type of CARs (e.g., both CD19 CAR and TSHR CAR).
   482. The method of embodiment 480 or 481, wherein a number of T cells expressing a single type of CAR molecule (e.g., CD19 CAR or TSHR CAR) is at least 2, 5, 10, or 20 times than T cells expressing more than one type of CARs (e.g., both CD19 CAR and TSHR CAR).
   482. The method of embodiment 480, wherein a number of T cells expressing a single type CAR molecule (e.g., CD19 CAR or TSHR CAR) is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) than T cells expressing more than one type of CARs (e.g., both CD19 CAR and TSHR CAR).
   483. A method of treating a subject having cancer, the method comprising: administering an effective amount of a pharmaceutical composition comprising CAR T cells prepared using the method of any one of embodiments 480-482 to the subject.
   484. A method of using the polynucleotide, modified cell, population of cells, fusion protein, composition, or methods of any one of embodiments 1 to 483 to treat a subject in need thereof.
   485. The method of embodiment 484, wherein the subject has cancer.

### EXAMPLES

### Cells Expressing Chimeric Receptors Establish Antitumor Effects in Patients with Relapsed/Refractory (R/R) Acute Lymphocytic Leukemia (ALL)

This clinical trial was designed to assess the safety and efficacy of infusing autologous T cells modified to express CD19 specific CAR/4-1BB/CD3-ζ into patients with R/R ALL. The inclusion criteria were as follows: 1) age not more than 60 years; 2) relapsed or refractory CD19+ALL; 3) relapsed allo-HSCT without evidence of graft versus host disease (GVHD) and not requiring immunosuppression therapy, and 4) measurable disease and adequate performance status and organ function. Patients with central nervous system leukemia (CNSL) were ineligible. The protocol was approved by the Institutional Review Board. All patients provided written informed consent.

The single-chain fragment variable (scFv) sequence-specific for CD19 was derived from Clone FMC63 (See Zola H. et al., Immunol Cell Biol 1991; 69:411-22). The 4-1BB co-stimulatory domain, CD3ζ signaling domain, and the hinge and transmembrane domains were generated. CART19-4-1BB vectors harboring anti-CD19 scFv (SEQ ID: 6) and the human 4-1BB and CD3ζ signaling domains were cloned into a lentiviral backbone as previously described (See Hu Y. Journal of Hematology & Oncology 2016; 9: 70).

Lentivirus was produced by transfecting 293T cells with CART19-4-1BB vectors and viral packaging plasmids which were frozen in −80° C. and thawed immediately before transduction. The lentiviral supernatant was harvested. CD3+ T cells were isolated and activated as described (See Kalos M. et al., Sci Transl Med 2011; 3:95ra73). The cells were then cultured in X-VIVO 15 medium (Lonza) containing 100 U/ml interleukin-2 (IL-2) and transduced with lentiviral supernatant at high multiplicity of infection (MOI) from 5:1 to 10:1 within 24-48 hours. The CAR transduced T cells (CD19CAR T cell) were obtained and cultured for 11 days. Three days before administration, fresh culture media were replaced. After that, no manipulation was conducted on the cells until transportation for infusion. The transduction efficiency was evaluated by flow cytometry (FACS) on day 5-7 after lentivirus transduction. The following anti-human antibodies were used: anti-hCD45 APC (BD Bioscience), anti-hCD3 FITC (BD Bioscience), biotin-labeled goat-anti-mouse IgG specific for F(ab′)2 fragment (Jackson immuno-Research, Cat #115-065-072) and PE streptavidin (BD Bioscience). Data acquisition were performed using a CytoFLEX flow cytometer (Beckman).

Prior to CD19CAR T infusion, flow cytometry (FACS) analysis of transduction efficiency and in vitro cytotoxicity assays of CD19 CAR T were performed for each patient as described herein. Additionally, CD19CAR T cultures were checked twice for possible contaminations by fungus, bacteria, mycoplasma, chlamydia, and endotoxin. PBMCs were obtained from patients by leukapheresis for CD19CAR T preparation on day 8, and the first day of CD19CAR T cell infusion was set as study day 0. Patients were given a conditioning treatment for lymphodepletion. Fludarabine- and cyclophosphamide-based conditioning treatment varied according to the tumor burden in the bone marrow (BM) and peripheral blood (PB). CD19CAR T cells were transfused directly to patients in escalating doses over a period of 3 consecutive days without any premedication. Each day CD19CAR T cells were transported to a hospital, washed, counted, checked for viability and then prepared for administration to patients, who were then observed closely for at least 2 hours. CRS was graded according to a revised grading system (See Lee D W. et al., Blood 2014; 124:188-95). Other toxicities during and after therapy were assessed according to the National Institutes of Health Common Terminology Criteria for Adverse Events Version 4.0 (http://ctep.cancer.gov/). Therapy responses were assessed by flow cytometry and morphological analysis. When possible, patients were assessed for chimeric gene expression levels. The response type was defined as minimal residual disease (MRD) negative, complete response, complete response with incomplete count recovery, stable disease, and progressive disease.

Serial BM and PB samples after CD19CAR T cell infusion were collected in K2EDTA BD vacutainer tubes (BD). The persistence of CD19CAR T cells from fresh PB and BM in patients was determined by FACS. Circulating CD19CAR T cell numbers per μl were calculated on the basis of measured absolute CD3+ T lymphocyte counts. Simultaneously, CAR DNA copies were evaluated as another method of determining CD19CAR T cell expansion and persistence. Genomic DNA was extracted using a QlAamp DNA Blood Mini Kit (Qiagen) from cryopreserved PB and BM. CD19CAR DNA copies were assessed by quantitative real-time PCR as described in the supplementary materials.

The levels of cytokines, such as IFN-γ, TNF-α, IL-4, IL-6, IL-10, IL-17, etc., in serum and cerebral spinal fluid (CSF) were measured in a multiplex format according to the manufacturer's instructions. Comparisons of continuous variables and risk factors that may influence variations in grade 3 or 4 CRS development were compared using the Mann-Whitney U test for 2 groups. Fisher's exact test was used to evaluate the influence of categorical variables on grade 3 CRS between 2 groups. Correlations were calculated using a rank-based Spearman test. Overall survival (OS) and leukemia-free survival (LFS) probabilities were determined by the Kaplan-Meier method using all enrolled patients to determine OS and those with MRD-negative responses for LFS. All quoted P values are two-sided, and P values less than 0.05 were considered statistically significant.

CD19+-RFP and Red Fluorescent Protein (RFP) were lentivirally transduced into K562 cells to produce CD19-RFP-K562 cells and K562-RFP cells, respectively. The cytotoxic activity of the CD19CAR T cells was measured before infusion by co-culture with the target cells, CD19-RFP-K562 cells or K562-RFP cells, at varying ratios of effector cell to a target cell (E:T). The target cells were plated into 96-well microwell plates (Nunc) at 10⁴ cells per well in 50 μl of RPMI 1640 media supplemented with 10% FBS (Gibco). The CD3/CD28 beads were removed, and effector T cells were mixed with target cells in the wells at the indicated E:T ratio. The total volume was 200 μl per well. After 24 hours of incubation, the cells were pipetted up and down in the 96-well microwell plates with a multi-channel pipettor to dissociate the cells into single-cell suspensions. The surviving RFP target cells in each well were photographed, and the number of surviving RFP target cells was counted and compared with those in the wells without effector cells. The cell death rate was calculated as (control-sample)/controlx100%. Supernatants were also collected and quantified using the human IFN-γ Valukine ELISA Kit (R&D Systems).

Genomic DNA was extracted from cryopreserved peripheral blood and bone marrow using a QlAamp DNA Blood Mini Kit (Qiagen). Quantitative real-time PCR was performed in triplicate using the ABI 2×TaqMan Universal Master Mix with AmpErase UNG (Applied Biosystems) in a 7500 real-time PCR system (Applied Biosystems). Copy numbers per microgram of genomic DNA were calculated from a standard curve of 10-fold serial dilutions of purified CAR plasmid containing 102-108 copies/μL. Amplification of an internal control gene was used for normalization of DNA quantities. Primers/probes specific for the CD19CAR transgene and an internal control gene were as previously described (See Gokbuget N. et al., Blood 2012; 120:2032-41 and O'Brien S. et al., J Clin Oncol 2013; 31:676-83).

Therapy response was assessed by flow cytometry and morphology. When possible, chimeric gene (CD19CAR) expression levels were assessed in the patients. The response type was defined as MRD-negative, complete response, complete response with incomplete count recovery, stable disease, and progressive disease, as previously described. MRD-negative was defined as less than 0.01% marrow blasts by flow cytometry. Complete response was defined as less than 5% marrow blasts, absence of circulating blasts, and no extramedullary sites of disease with absolute neutrophil counts of 1000 per μL or more and platelets 100,000 per μL or more. Complete response with incomplete count recovery was defined as a complete response with cytopenia. Stable disease was defined as a disease that did not meet the criteria for complete response, complete response with incomplete count recovery, or progressive disease. Progressive disease was defined as worse M status or no change in M status but a greater than 50% increase in absolute peripheral blast count. After CD19CAR T cell therapy, patients were followed up every week and underwent bone marrow examination including morphology, MRD status, chimeric gene expression and a CAR T cell count every 4 weeks.

The samples were collected in gel tubes and stored at 4° C. until centrifugation later the same day. All blood and CSF samples were then centrifuged at 5000 rpm for 6 minutes. The supernatants were transferred for subsequent analysis. The BD Cytometric Bead Array Human Th1/Th2/Th17 Cytokine Kit (BD Biosciences), FCAP Array v3.0 software (BD Biosciences), and a BD FACS CANTO II (BD Biosciences) were used for the measurement and analysis of the concentrations of the cytokines such as IL-2, IL-4, IL-6, IL-10, IL-17A, IFN-γ, and TNF-α. according to the manufacturer's instructions.

Erythrocyte-lysed whole BM samples were used for immunophenotyping on the day of bone marrow aspiration. Antigen expression of blast cells was systematically analyzed by flow cytometry (FACSCalibur flow cytometer, BD Biosciences, San Jose, Calif.) using four-color combinations of monoclonal antibodies (mAbs) with fluorescein isothiocyanate (FITC), phycoerythrin (PE), allophycocyanin (APC), and phycoerythrin-cyanin 7 (PE-Cy7). Cell-Quest software (Becton Dickinson Biosciences) was used for data analysis. Monoclonal antibodies were purchased from the following manufacturers: BD Biosciences, CD10-APC, CD19-FITC, CD22-PE, CD34-PE, CD45-PE-Cy7, cyCD79a-PE, surface immunoglobulin (slg) M-PE, cytoplasmic immunoglobulin (clg) M-APC; and Beckman Coulter, CD20-APC, slg-Lamda-FITC, slg-Kappa-APC.

For the investigation of Minimal residual disease (MRD), the combination of mAbs was based on the aberrant phenotypes of leukemic blasts at diagnosis individually. The MRD results were presented as the percentage of cells with aberrant phenotypes among nucleated cells. A sensitivity of 0.01% was achieved in all samples analyzed. The instrument setup was calibrated daily by analyzing Calibrite™ beads and standard blood samples (BD™ Multi-Check Control from BD Biosciences or CD-Chex™ Plus from Streck, Inc.) for quality control.

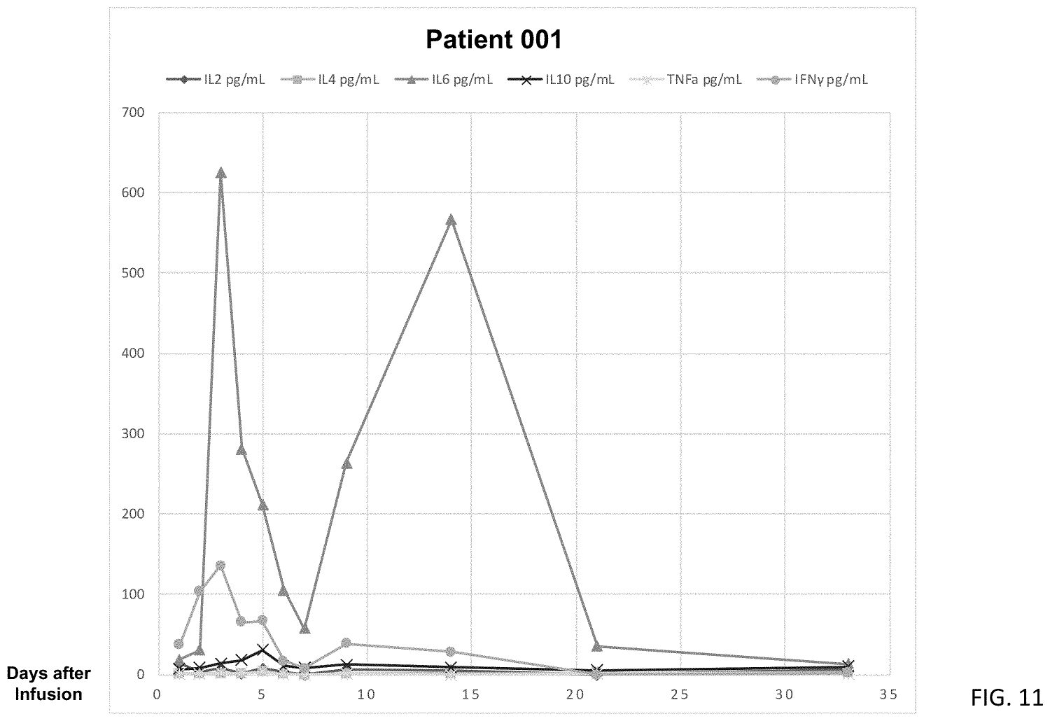

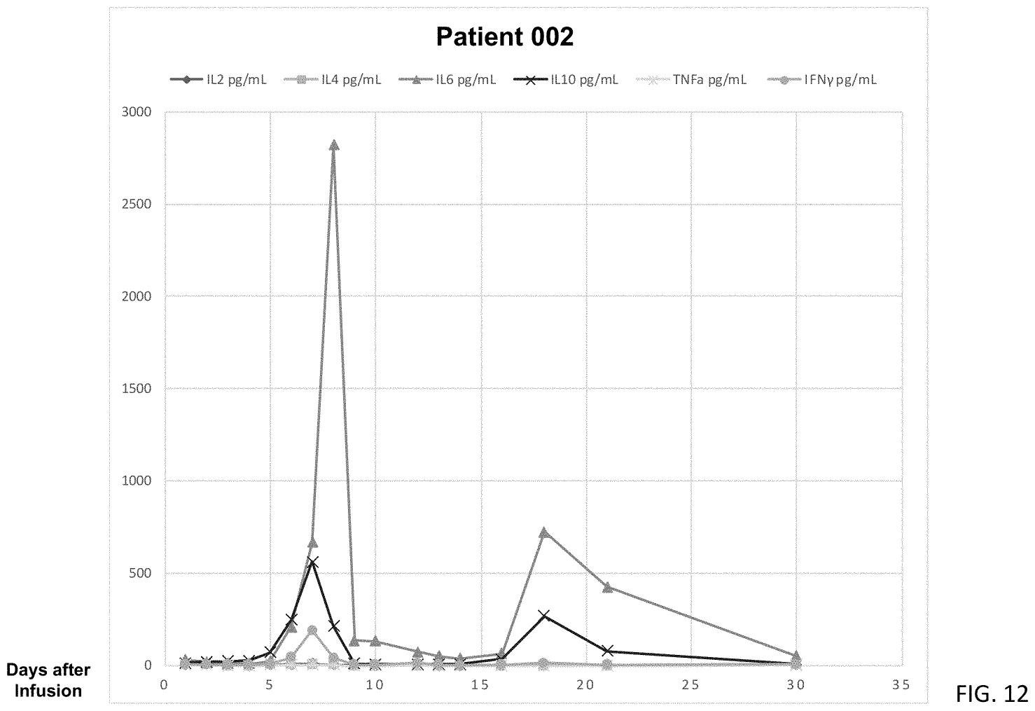

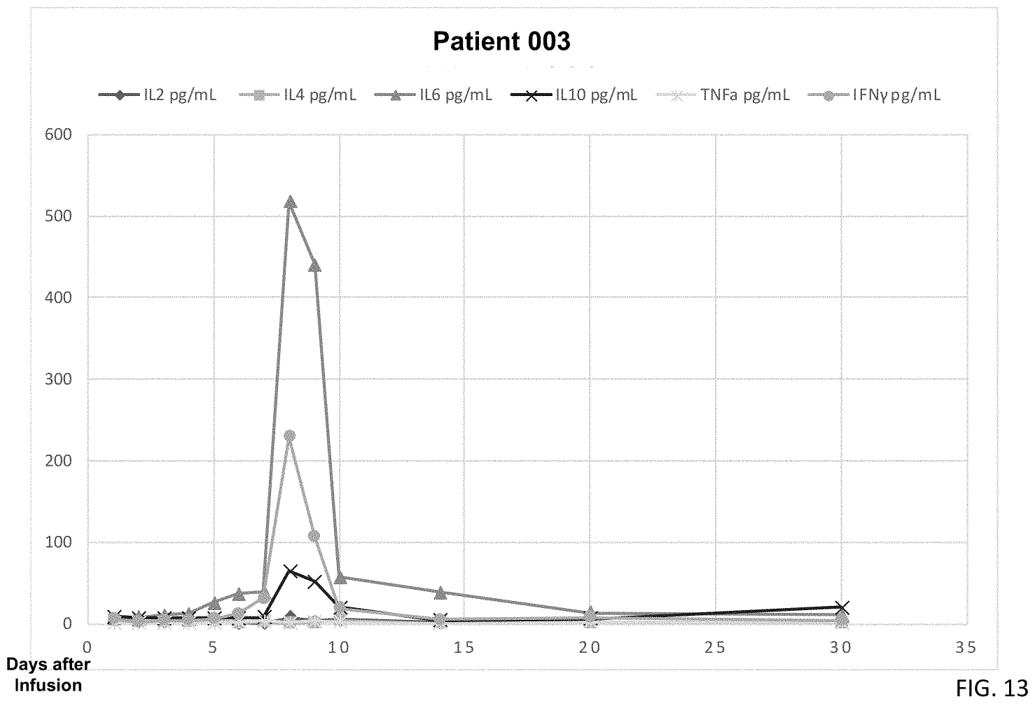

Conventional CAR T cells did not seem to be effective for treating solid tumor. Thus, the data of treating blood tumors using CAR T cells may be used to help enhance treatment of solid tumors using CAR T cells. Here, three patients with relapsed/refractory Chronic Lymphocytic Leukemia (R/R ALL) were treated with CD19CAR T cells (See Table 5). These results demonstrate that T cells expressing CD19edCAR establish antitumor effects in patients with R/R ALL. In addition, IL-6, IFNγ, and IL-10 were significantly elevated in the blood of the patients after the infusion of CD19CAR T cells, as compared to other factors (See FIGS. 11-13). Therefore, one or more of these cytokines may be expressed in CAR T cells to enhance treatment of solid tumors. Given that IL-10 inhibits anti-tumor activities and IFNγ reduces M2 macrophages (See Example below), IL-6 and IFNγ were first selected to be expressed or overexpressed in modified T cells for treating solid tumors and lymphoma. Further, modified cells having inducible expression of IL-6 and/or IFNγ were further generated to reduce or avoid risks of severe adverse reactions caused by the modified cells on subjects.

TABLE 5

| Patient | Tumor | CART Dosage | | CRS |
|---|---|---|---|---|
| ID | type | (×10E6/kg) | Response | grade |
| 001 | B-ALL | 4.8 | MRD (−) | 3 |
| 002 | B-ALL | 1.7 | MRD (−) | 4 |
| 003 | B-ALL | 2.8 | MRD (−) | 3 |

### Reducing M2 Macrophages by Modified Cells Expressing IFNγ

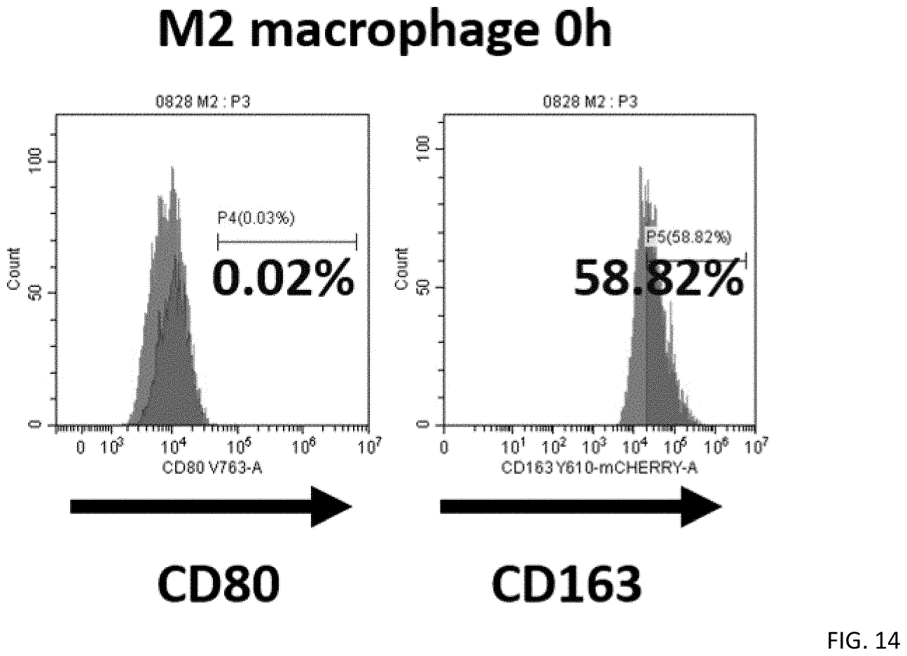

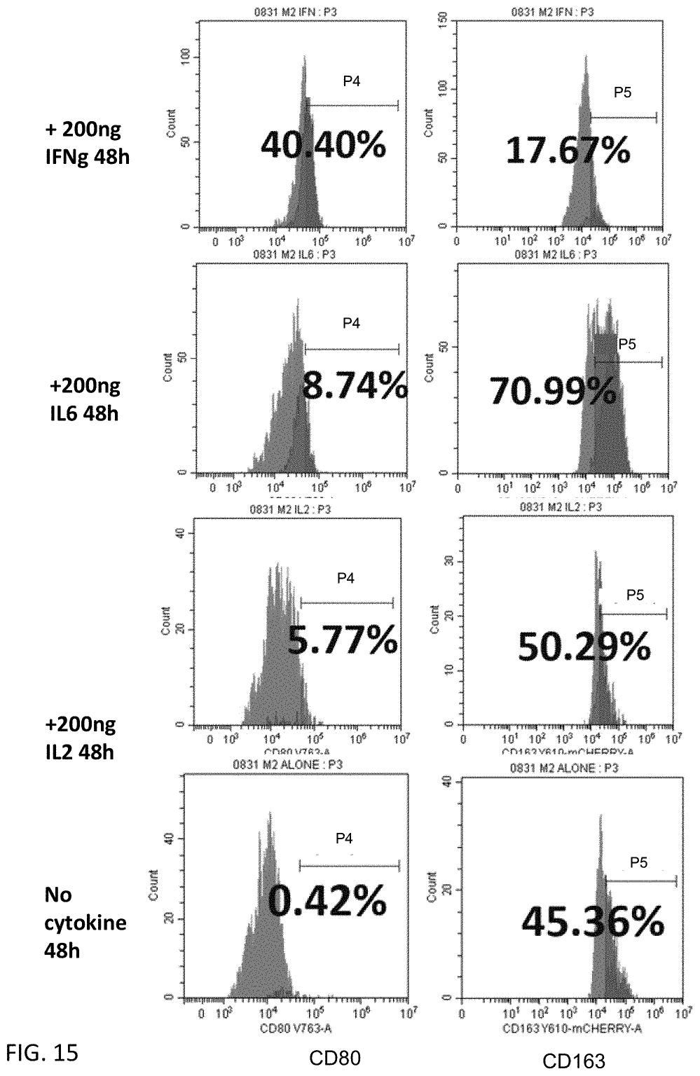

It has been reported that there are many kinds of cells in solid tumor tissues, including tumor cells and some immune cells. Among them, macrophages are the main cells in immune cells, mostly M2 macrophages. This solid tumor M2 type macrophages are inside tumor tissues and continuously secrete cytokines to nourish the tumor cells. FIGS. 14 and 15 show cytometry assay results of macrophage culturing and cell phenotype changes in response to cytokine added. M2 macrophages express CD613 but not CD80, while M1 macrophages express CD80 but not 163. Mononuclear cells were selected from the blood of healthy volunteers by sorting with CD14 microbeads. The cells were incubated with M2 medium (containing IL4) for 3 days at a density of 1×10⁶ cells/ml. After 3 days, the medium was changed, and the cell culture density was adjusted to 1×10⁶ cells/ml. After 4-6 days, 1×10⁶ cells were taken out, and the expression of CD14 and CD163 was detected by flow cytometry (FIG. 14). 1×10⁶ CAR+ cells were removed from the same volunteers at Day 7 and were added to 6-well plates. 4×10⁵ cells of M2 macrophage were added to each well, then 200 ng of IFNγ, 200 ng IL6, and 200 ng IL2 were added to each well, and the cells were cultured for 2 days. After 62 days, 1×10⁶ cells were removed, and the expression of CD14 and CD163 was detected by flow cytometry. FIG. 15 shows the expression of macrophage with CD163 or CD80 in various groups. These results showed that M2 macrophages can be converted to M1 macrophages, and cytokines such as IFNγ reduced the number of M2 macrophages and caused M2 macrophages to convert to M1 macrophages.

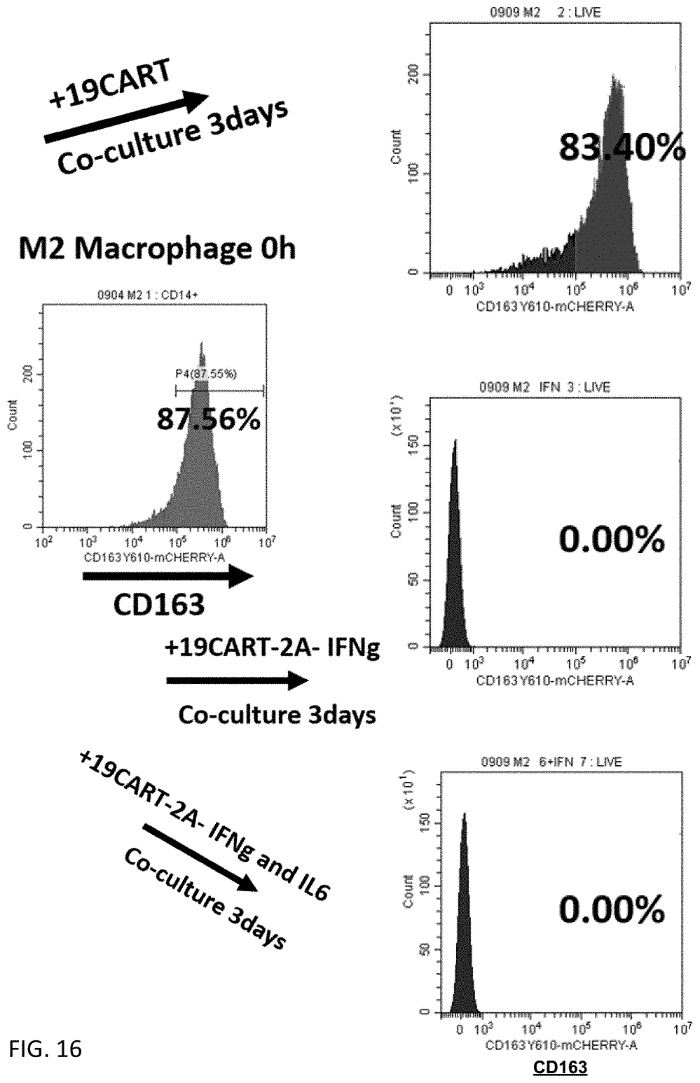

FIG. 16 shows cytometry assay results of macrophages co-cultured with various CAR T cells expressing IFN-γ and/or IL-6. Mononuclear cells were selected from the blood of healthy volunteers by sorting with CD14 microbeads. The cells were incubated with M2 medium (containing IL4) for 3 days at a density of 1×10⁶ cells/ml. After 3 days, the medium was changed, and the cell culture density was adjusted to 1×10⁶ cells/ml. After 4-6 days, 1×10⁶ cells were removed, and the expression of CD14 and CD163 was detected by flow cytometry. 1×10⁶ CAR+ cells were removed from the same volunteers on Day 7 and were added to 6-well plates. 4×10⁵ cells of M2 macrophages were added to each well for 3 days. After 63 days, 1×10⁶ cells were removed, and the expression of CD14 and CD163 was detected by flow cytometry. These results showed that CAR T cells expressing IFNγ reduced M2 macrophages and converted them to M1 macrophages.

### Modified Cells Expressing Cytokines and In Vitro Assay

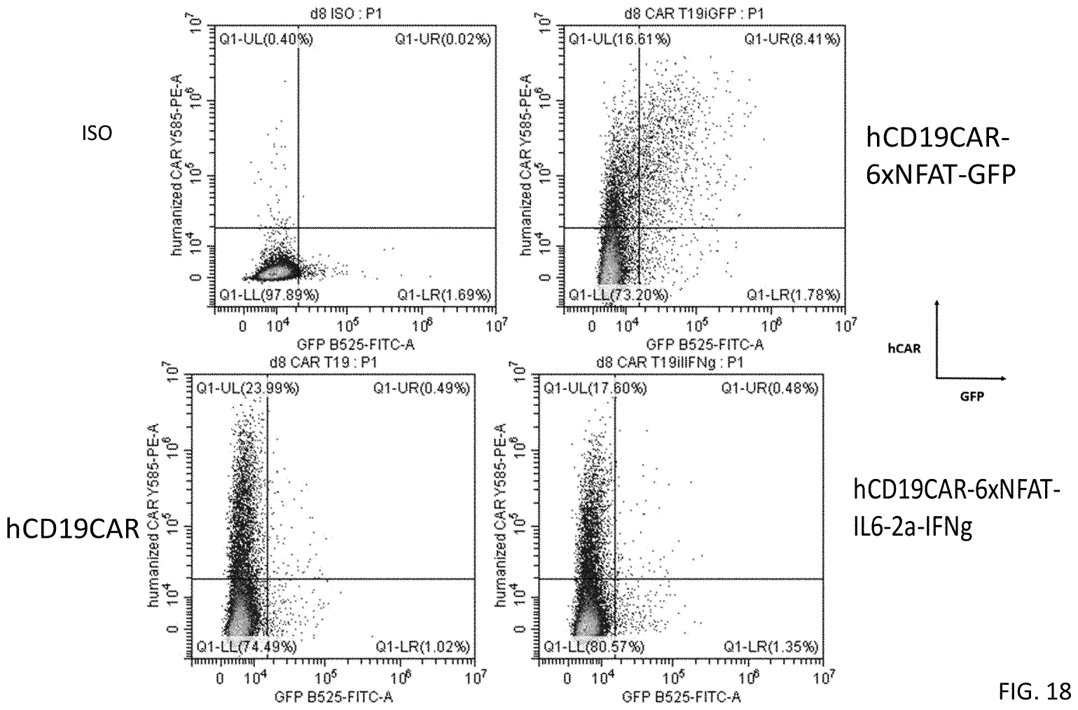

FIG. 18 shows copy numbers of T cells expressing various proteins. Day 1, T cells from a healthy donor were sorted and activated using CD3/CD28 beads. Day 2, 10⁵ cells were transfected with vectors 1230 (1230 represents h19CAR) (MOI 10:1), 6205 (hCD19CAR-GFP) (MOI 60:1), and 6221 (hCD19CAR-6xNFAT-IL6-2a-IFNg) (MOI 60:1), respectively. On Day 3, cell media were changed. On Day 4, cell numbers were counted. On Days 5, 6, and 8, assays for measuring culturing factors, CAR copy number, phenotype, and expression were conducted. Day 8, toxicity assays were performed, and culturing factors were detected. The copy numbers are provided in Table 6 below. In this and the following examples, the sequence for NFAT is SEQ ID NO 469; for TSHRCAR is SEQ ID NO: 279; for scFv of TSHRCAR is SEQ ID NO: 8; for scFv of CD19CAR is SEQ ID 5; for IL6 is SEQ ID NO: 287; for peptide 2A is SEQ ID NO: 327; and for IFNγ is SEQ ID NO: 328. The sequences of other components may be found in Table 2. Table 6 shows copy numbers of CAR per CAR T cell.

TABLE 6

| Type of T cells | 6205 | 6221 | 1230 |
|---|---|---|---|
| Day 8/per CAR-T | 1.02728 | 0.66634 | 1.3325 |

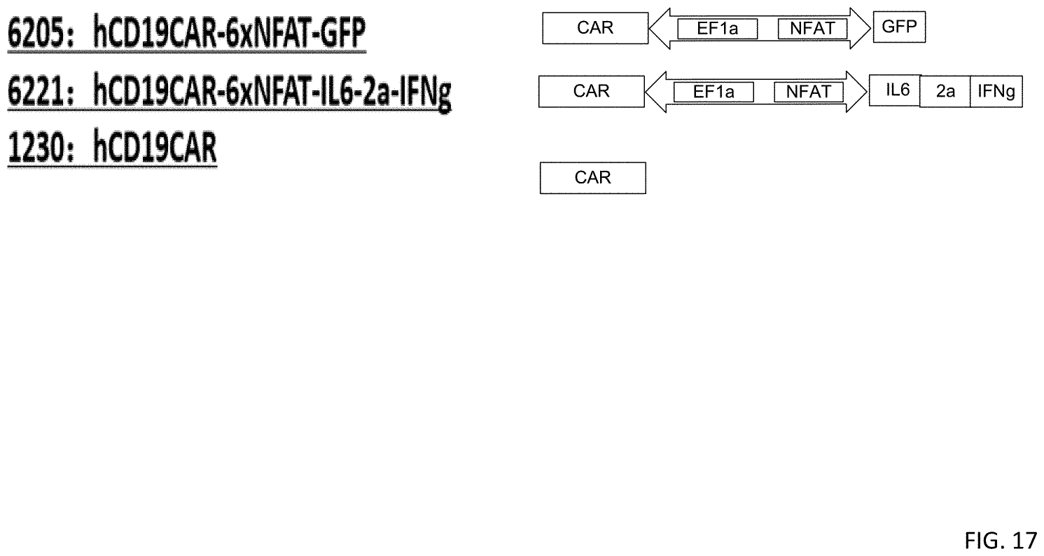

FIG. 18 shows flow cytometry assay results of T cells expressing various proteins shown in FIG. 17. On Day 0, peripheral blood of healthy volunteers was taken, and CD3+ T cells were sorted from the sample. CD3/CD28 Dynabeads were added to the stored CD3+ T cells at a 1:1 ratio. On Day 2, T cells were transfected using lentivirus including the following vectors. CD19CAR was infected according to the infection ratio of MOI=10:1; hCD19CAR, hCD19CAR-6xNFAT-GFP, hCD19CAR-6xNFAT-GFP, hCD19CAR-6xNFAT-IL6-2a-IFNγ cells were infected according to the infection ratio of MOI=60:1. On Day 3, the media were changed, the lentiviruses were removed, and the cells were resuspended in fresh medium. On Day 7, flow cytometry assays were used to detect CAR expression. CD19CAR is a humanized antibody and is therefore detected with a human CAR antibody. CD19CAR expression was 23.99%; hCD19CAR-6xNFAT-GFP CAR expression was 25%, and hCD19CAR-6xNFAT-IL6-2a-IFNg CAR expression was 17.6%. Flow cytometry assay was performed using human CAR antibody to detect the expression intensity and expression level of CAR.

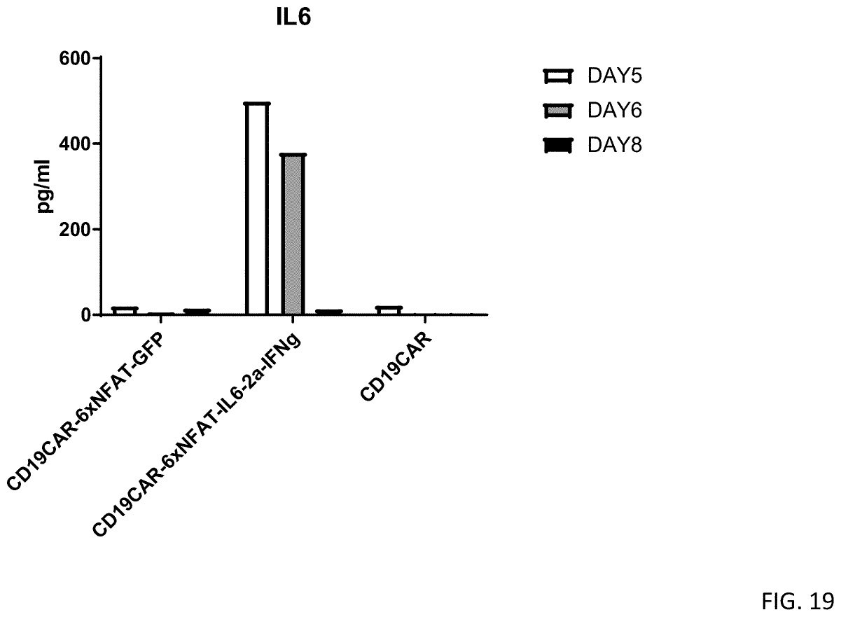

FIG. 19 shows IL6 release in response to CD3/CD28 Dynabeads activation. On Day 0, peripheral blood of healthy volunteers was taken and CD3+ T cells were sorted from the sample. CD3/CD28 Dynabeads were added to the stored CD3+ cells at a 1:1 ratio. On Day 2, T cells were transfected using lentivirus including the following vectors. CD19CAR was infected according to the infection ratio of MOI=10:1; hCD19CAR, hCD19CAR-6xNFAT-GFP, hCD19CAR-6xNFAT-GFP, hCD19CAR-6xNFAT-IL6-2a-IFNg cells were infected according to the infection ratio of MOI=60:1. On Day 3, the media were changed, and the lentiviruses were removed, and the cells were resuspended in fresh medium. On Day 5, 6, and 8, 200 ul of cell supernatant was used to detect the release of IL6. As shown in FIG. 19, on Day 5, 6, and 8, 200 ul of cell supernatant was taken from the media and used to detect the release of IL6 factor. The amount of IL6 released per 10⁴ of CD19CAR and CD19CAR-6xNFAT-GFP was 0-10 μg/ml on Day 5, 6, and 8. 10⁴ CD19CAR-6xNFAT-IL6-2A-IFNg had IL6 release of 498 pg/ml and 378 pg/ml on Day 5 and 6, and the released amount of IL6 on Day 8 was 9.8 pg/ml. On Days 5 and 6, cells were cultured with CD3/CD28 Dynabeads such that the cells were activated, and NFAT element was activated, and the transcription of IL6 was enabled, causing IL6 to be released. However, on Day 8, the effect of Dynabeads stimulation was dropped to a lower level, and the cells were not activated. Therefore, the NFAT element was turned off, and the transcription of IL6 was disabled. Thus, IL6 was not released. When T cells are stimulated by CD3/28 Dynabeads, the cells are activated for a short period of time. The NFAT element induces transcriptional translation of the gene in response to the activation, and the corresponding genes are expressed. When the cells are at rest or at a lower level of activation, the NFAT element does not initiate transcriptional translation of the gene of interest. Therefore, whether the NFAT element is active or not and whether the induced gene is expressed can be determined by the expression of the target gene in the activated and inactivated state of the CAR T cell.

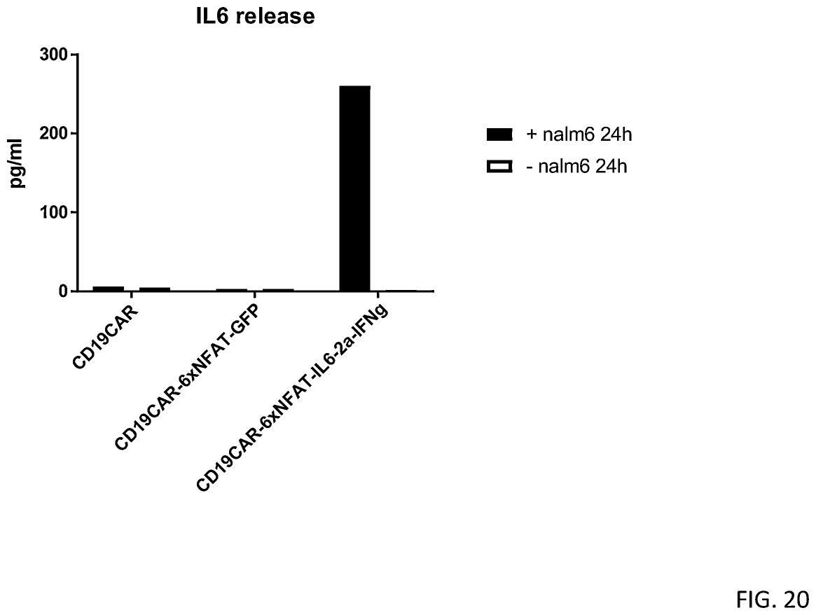

FIG. 20 shows IL6 release in response to co-culturing with Nalm6 cells (see Table 7 below). The cells were cultured to Day 8 and then were leveled with NT cells to differentiate the CAR ratio of CD19CAR T cells and CD19CAR-6xNFAT-GFP to cells of CD19CAR-6xNFAT-IL6-2a-IFNg. 10⁴ CAR+ cells were co-cultured with 10⁴ 4 Nalm-6 cells or cultured separately. After 24 hours, the supernatant was collected, and the amount of IL6 released was measured. The cells were co-cultured with Nalm6 cells and were in an activated state. Thus, the NFAT element initiated transcription of IL6 in an activated state, allowing IL6 to be released. When the CAR T cells are co-cultured with the cells expressing the antigen to which the CAR binds (target cells), since the CAR T cells recognize the membrane proteins on the surface of the tumor cells, the cells are activated. The NFAT element initiates transcriptional translation of the gene in response to activation, and the corresponding gene is expressed. When the cell is at rest or when the activation level is low, the NFAT element does not initiate transcriptional translation of the gene of interest. Therefore, whether the NFAT element is active or not and whether the induced gene is expressed can be determined by the expression of the target gene in the activated and inactivated state of the CAR T cell.

TABLE 7

| | | CD19CAR- | CD19CAR- |
|---|---|---|---|
| | | 6xNFAT- | 6xNFAT- |
| CART cells 10⁴ | CD19CAR-T | GFP | IL6-2a-IFNg |
| Co-cultured cells | Nalm6 | Nalm6 | Nalm6 |
| (25 hours) 10⁴ | | | |
| IL6 released: CAR | 0-10 pg/ml | 0-10 pg/ml | 260 pg/ml |
| T cells co-cultured | | | |
| with Nalm6 | | | |
| IL6 released: CAR | 0-10 pg/ml | 0-10 pg/ml | 0-10 pg/ml |
| T cells cultured | | | |
| alone | | | |

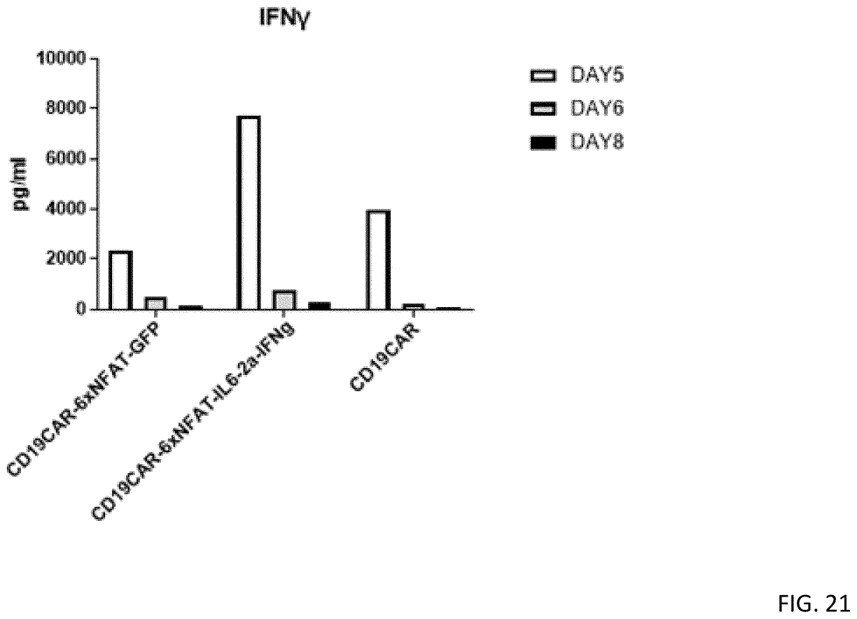

FIG. 21 shows IFNγ (i.e., IFNg) release in response to CD3/CD28 Dynabeads activation. On day 0, peripheral blood of healthy volunteers was taken, and CD3+ T cells were sorted. CD3/CD28 Dynabeads were added in a 1:1 ratio. On Day 2, T cells were transfected using lentivirus including the following vectors. CD19CAR T cells were infected according to the infection ratio of MOI=10:1; hCD19CAR, hCD19CAR-6xNFAT-GFP, hCD19CAR-6xNFAT-GFP, hCD19CAR-6xNFAT-IL6-2a-IFNγ cells were infected according to the infection ratio of MOI=60:1. The lentiviruses were removed, and the cells were resuspended in fresh medium. On Days 5, 6, and 8, 200 ul of the cell supernatant was used to detect the release of IFNγ. On Days 5 and 6, cells were cultured with CD3/CD28 Dynabeads such that the cells were activated, and NFAT element was activated, which enabled the transcription of IFNγ and the release of IFNγ. However, on Day 8, the effect of Dynabeads stimulation has dropped to a lower level, and the cells were no longer activated. Therefore, the NFAT element was turned off, and transcription of IFNγ was disabled. Thus, IFNγ was not released. When T cells are stimulated by CD3/28 Dynabeads, the cells are activated for a short period of time. The NFAT element initiates transcriptional translation of the gene in response to the activation, and the corresponding genes are expressed. When the cells are at rest or at a lower level of activation, the NFAT element does not initiate transcriptional translation of the gene of interest. Therefore, whether the NFAT element is active or not and whether the induced gene is expressed can be determined by the expression of the target gene in the activated and inactivated state of the CAR T cell.

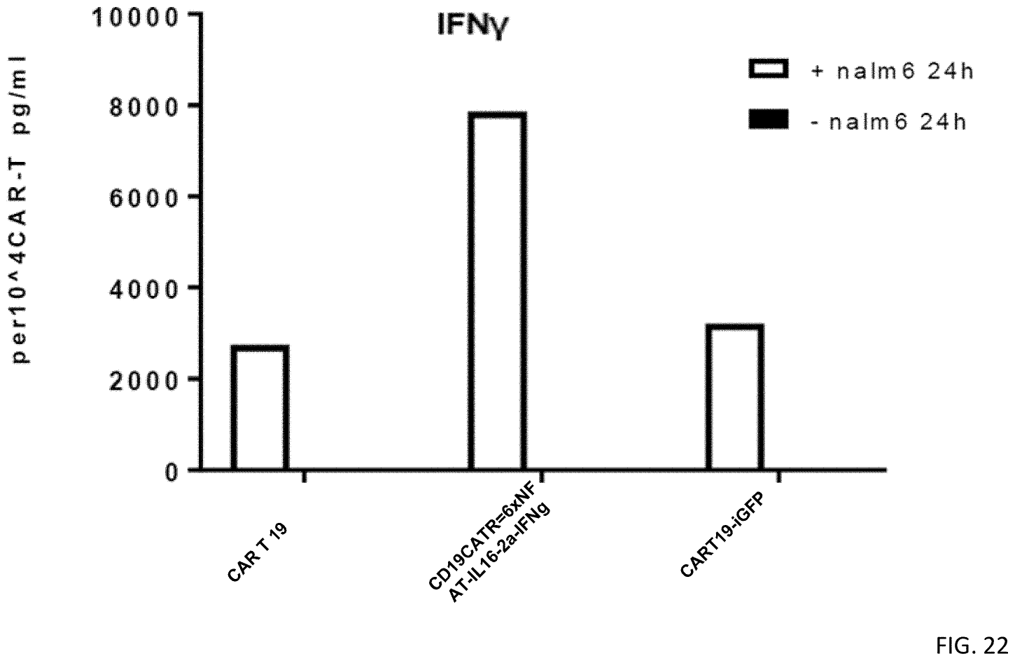

FIG. 22 shows IFNγ release in response to co-culturing with Nalm6 cells (See Table below 8). The cells were cultured to Day 8 and then were leveled with NT cells to differentiate the CAR ratio of CD19 CAR T cells and CD19CAR-6xNFAT-GFP to cells of CD19CAR-6xNFAT-IL6-2a-IFNg. 10⁴ CAR+ cells were co-cultured with 10⁴ Nalm-6 cells or cultured separately. After 24 hours, the supernatant was collected, and the amount of IFNγ released was measured. The cells were co-cultured with Nalm6 cells and were in an activated state. Thus, the NFAT element initiated transcription of IFNγ in an activated state, allowing IFNγ to be released. When the CAR T cells are co-cultured with the target cells, the cells are activated because the CAR T cells recognize the membrane proteins on the surface of the tumor cells. The NFAT element initiates transcriptional translation of the gene to be expressed due to the activation, and the corresponding gene is expressed. When the cell is at rest or when the activation level is low, the NFAT element does not initiate transcriptional translation of the gene of interest. Therefore, whether the NFAT element is active or not and whether the induced gene is expressed can be determined by the expression of the target gene in the activated and inactivated state of the CAR T cell.

TABLE 8

| | | CD19CAR- | CD19CAR- |
|---|---|---|---|
| CART cells | | 6xNFAT- | 6xNFAT- |
| 10e4 | CD19CAR-T | GFP | IL6-2a- IFNγ |
| Co-cultured | Nalm6 | Nalm6 | Nalm6 |
| cells (25 hours) | | | |
| 10e4 | | | |
| IFNγ released: | 2400-3200 pg/ml | 2400-3200 pg/ml | 7900 pg/ml |
| CAR T cells | | | |
| co-cultured | | | |
| with Nalm6 | | | |
| IFNγ released: | 2400-3200 pg/ml | 2400-3200 pg/ml | 2400-3200 pg/ml |
| CAR T cells | | | |
| cultured | | | |
| alone | | | |

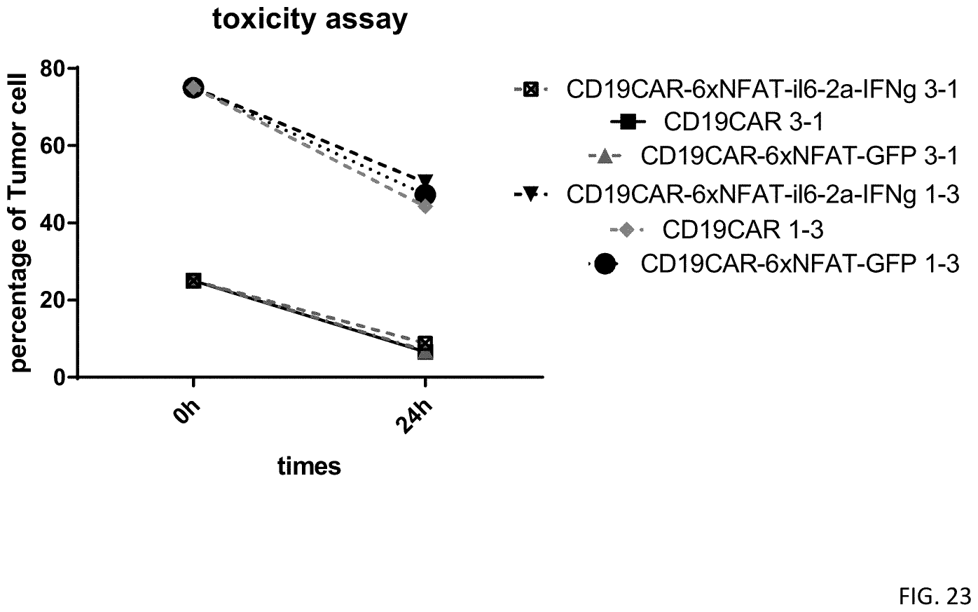

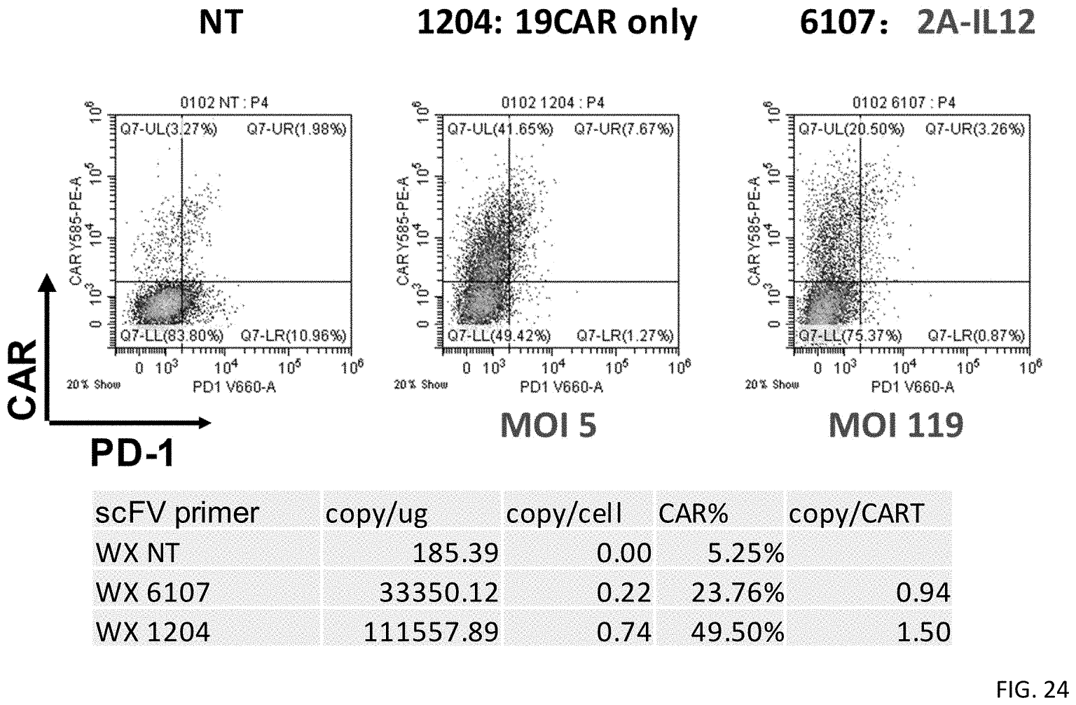

FIG. 23 shows toxicity assay with respect to CAR T cells. T cells from a healthy donor were cultured to Day 8 and then were leveled with NT cells to differentiate the CAR ratio of CD19 CAR T cells and CD19CAR-6xNFAT-GFP to cells of CD19CAR-6xNFAT-IL6-2a-IFNγ. 3×10⁴ CAR+ cells were co-cultured with 10⁴ Nalm-6 cells and 904 Nalm-6 cells, respectively. The residual of Nalm6 cells was detected after 24 hours. CD19CAR T cells, CD19CAR-6xNFAT-GFP cells, and CD19CAR-6xNFAT-IL6-2a-IFNg cells were co-cultured with Nalm6 cells in different ratios. There were no significant differences among the 3 cell groups. After the cells of CD19CAR, CD19CAR-6xNFAT-GFP, and CD19CAR-6xNFAT-IL6-2a-IFNγ were activated by the tumor, the T cells executed a killing function and acted on the target cells to cause the target cells to be killed.

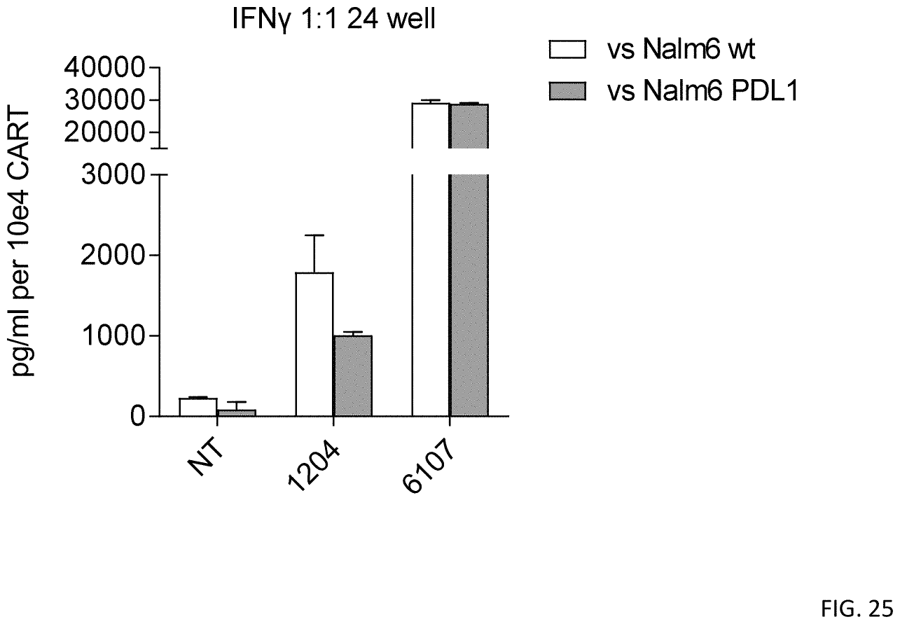

FIG. 25 show IFNγ release, respectively, in response to co-culturing with Nalm6 cells. On Day 0, peripheral blood T cells were obtained from volunteers and stimulated using Dynabeads at 1:1 ratio. On Day1, the cells were infected with lentiviral vectors. On Day 2, the media were changed. On Day 7, flow cytometry assays were used to detected CAR expression and CAR copy numbers. 1204 represents hCD19CAR cells, and 6107 represents hCD19CAR-2A-IL12 cells which express IL12 continuously. The CAR expression was normalized to 17%. 10⁴ CAR positive cells and 10⁴ Nalm6 or Nalm6-PDL1 tumor cells were co-cultured in 24-well plates for 24 hours, and the supernatant was assayed for IFNγ. 1204 had 49.50% CAR expression and 1.5 copies per CAR T cell, and 6107 had 23.76% CAR expression and 0.94 copies per CAR T cell. CAR was normalized to 17% for co-culture. Co-culture results, as shown in the histogram, showed that IFNγ produced by Nalm6-PDL1 stimulation for 1204 was about half that stimulated by Nalm6 wt because of the inhibitory effect of PDL1 on T cells. The release of IFNγ from 6107 reached about 10 times that of 1230, demonstrating that IL12 released by CAR T significantly promoted IFNγ release.

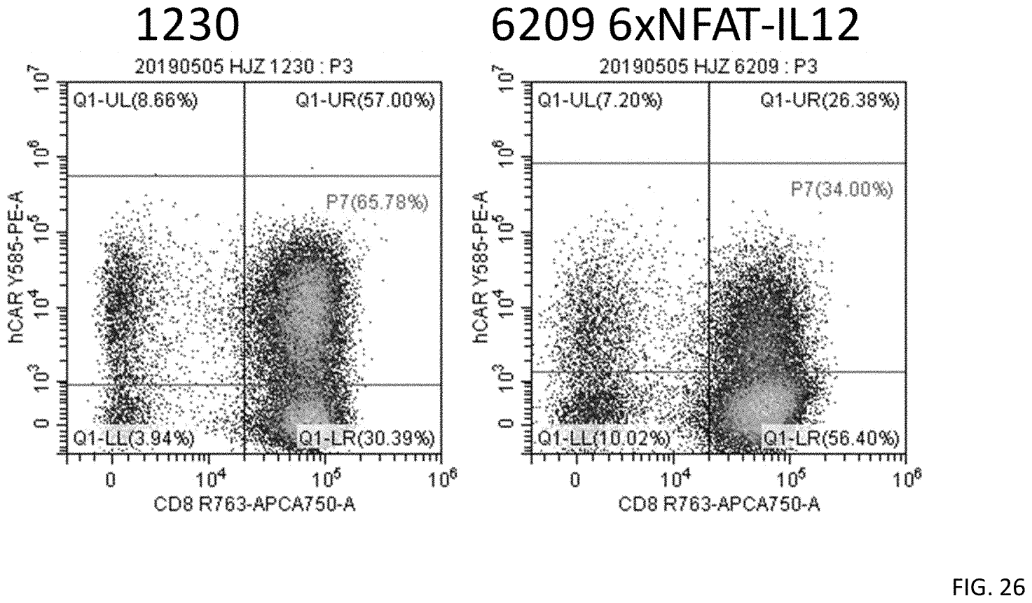

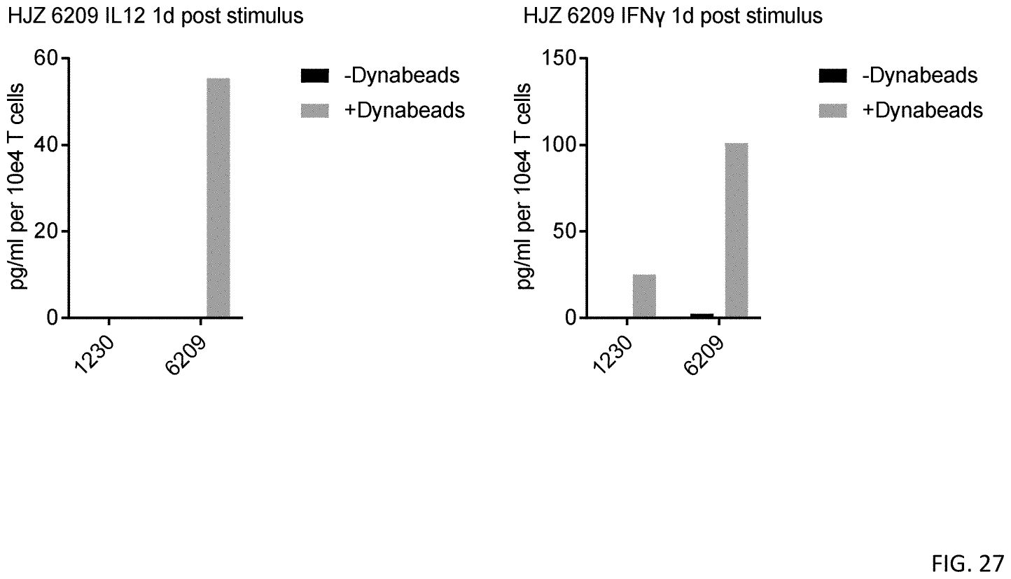

FIGS. 26 and 27 show IL12 and IFNγ release in response to CD3/CD28 Dynabeads activation. On Day 0, peripheral blood T cells were obtained from volunteers and stimulated using Dynabeads at 1:1 ratio. On Day 1, the cells were infected with lentiviral vectors. On Day 2, the media were changed. On day 9, flow cytometry was used to detect CAR expression. 1230 represents h19CAR, and 6209 represents h19CAR-6xNFAT-IL12 (conditional release of IL12 under T cell activation). The CAR expression was normalized to 30%. The same number of cells were stimulated by Dynabeads for 24 hours at a ratio of 1:3, and the supernatant was assayed for IL12 and IFNγ. There was 65% CAR expression in 1230 and 34% CAR expression in 6209. After normalization to 30%, CD3/CD28 Dynabeads were added. After 24 hours, the supernatant was assayed to collect information of cytokines, and results were presented as a histogram. Under the stimulation of Dynabeads, 6209 released an average of 55 μg of IL12 per 10⁴ of T cells. 1230 did not release IL12 with or without stimulation, and 6209 did not release IL12 without stimulation. This indicates that, originally, NFAT activated IL12 transcription under Dynabeads stimulation, as shown on the left. IFN was released as shown on the right. Both CAR T cells did not release IFNγ without stimulation, and 6209 released more IFNγ under Dynabeads stimulation, indicating that IL12 synergizes with 6209 CART cells to release more IFNγ.

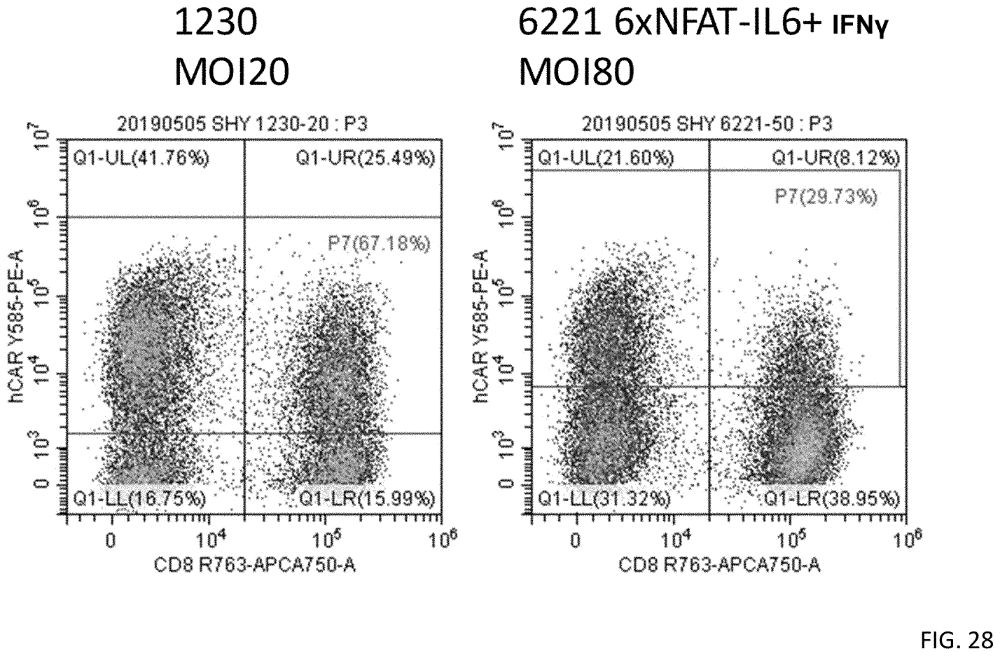

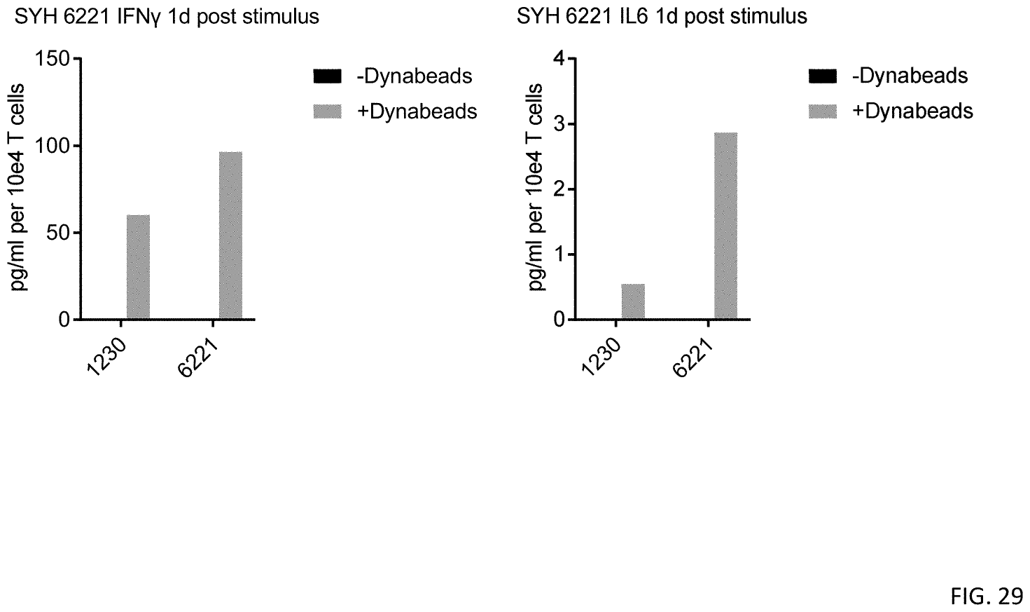

FIGS. 28 and 29 show IL6 and IFNγ release in response to CD3/CD28 Dynabeads activation. On Day 0, peripheral blood T cells were obtained from volunteers and stimulated using Dynabeads at 1:1 ratio. On Day 1, the cells were infected with lentiviral vectors. On Day 2, the media were changed. On day 7, flow cytometry was used to detect CAR expression. 1230 represents hCD19CAR, and 6221 represents hCD19CAR-6xNFAT-IL6-2A-IFNγ (conditional release of IL6 and IFNγ under T cell activation). The CAR expression was normalized to 12.66%. The same number of cells were stimulated by Dynabeads for 24 hours at a ratio of 1:3, and the supernatant was assayed for IL6 and IFNγ. There was 67% CAR expression in 1230, and 29% CAR expression in 6221. The CAR expression was normalized 12.66%, and Dynabeads were added. After 24 hours, the supernatant was assayed to measure the cytokines. The results of the assay were presented as a histogram. Both cells were free of IL6 or IFNγ release without Dynabead activation. After addition of Dynabeads, 6221 released significantly more IL6, and IFNγ (based on the average number of pg released per 10⁴ T cells).

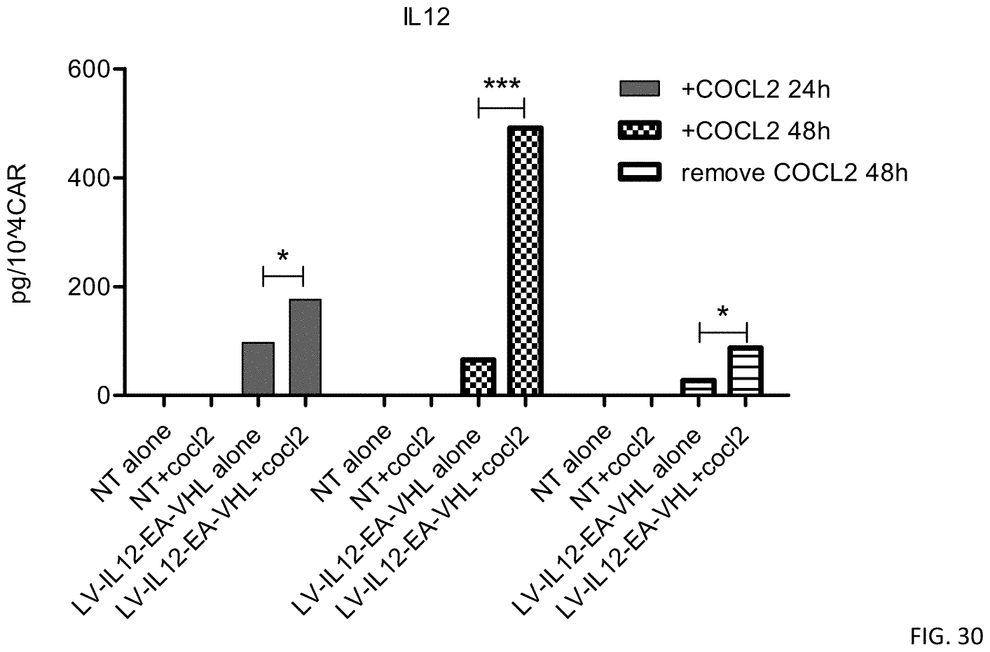

FIGS. 30-38 show the induction of expression of cytokines by CAR T cells. The experiments were conducted using various solid tumor markers (e.g., TSHR, ACPP, and GUCY2C). The data show that activated CAR T cells can induce the expression of IL-6, IL-12, and/or IFNγ, and the expression of various factors is significantly lower under aerobic conditions than under anaerobic conditions. It has also been shown that the regulated expression of cytokines can up-regulate factors related to the killing function of other cytokines (e.g., IFNγ and GZMB). Various vectors as well as cells and their references described in FIGS. 30-38 are listed in Table 9.

TABLE 9

| Name | Construction | Name | Construction | Name | Construction |
|---|---|---|---|---|---|
| 1604 | TSHR-BBz | 6503 | ACPP-BBZ | 6701 | GUCY2C-bbz |
| 6918 | TSHR-28Z | 6521 | ACPP-bbz-NFAT6x-IL6 | 6923 | GUCY2C-28z |
| 6270 | TSHR-bbz- | 6523 | ACPP-bbz-NFAT6x- | 6278 | GUCY2C-bbz- |
| | NFAT6X-IL12- | | IFNg-2a-IL6 | | NFAT6X-IL12-VHL |
| | VHL | | | | |
| 6271 | TSHR-bbz- | 6524 | ACPP-bbz-NFAT6x- | 6279 | GUCY2C-28z- |
| | NFAT6X-IL12- | | IFNg-2a-IL6VHL | | NFAT6X-IL12-VHL |
| | VHL | | | | |
| 6526 | ACPP-bbz- | 6525 | ACPP-bbz-NFAT6x- | | |
| | NFAT6x-IL6-2a- | | IL6-2a-IL12 | | |
| | IL12VHL | | | | |

Note:
BBZ: 4-1BB and CD3 zeta
28Z: CD28 and CD3 zeta

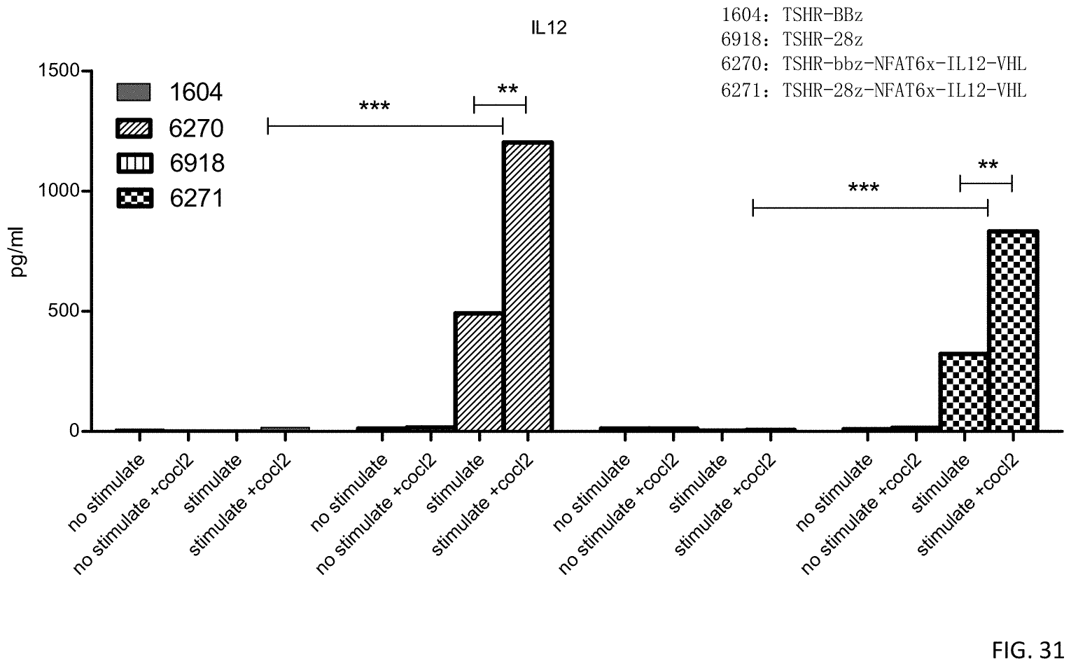

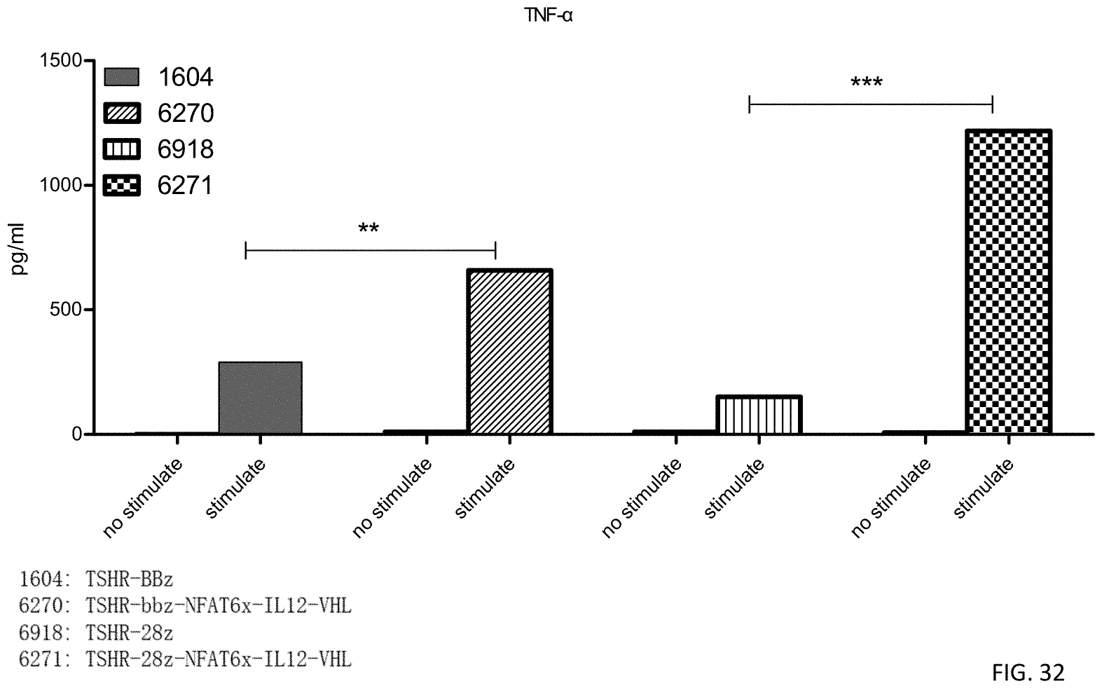

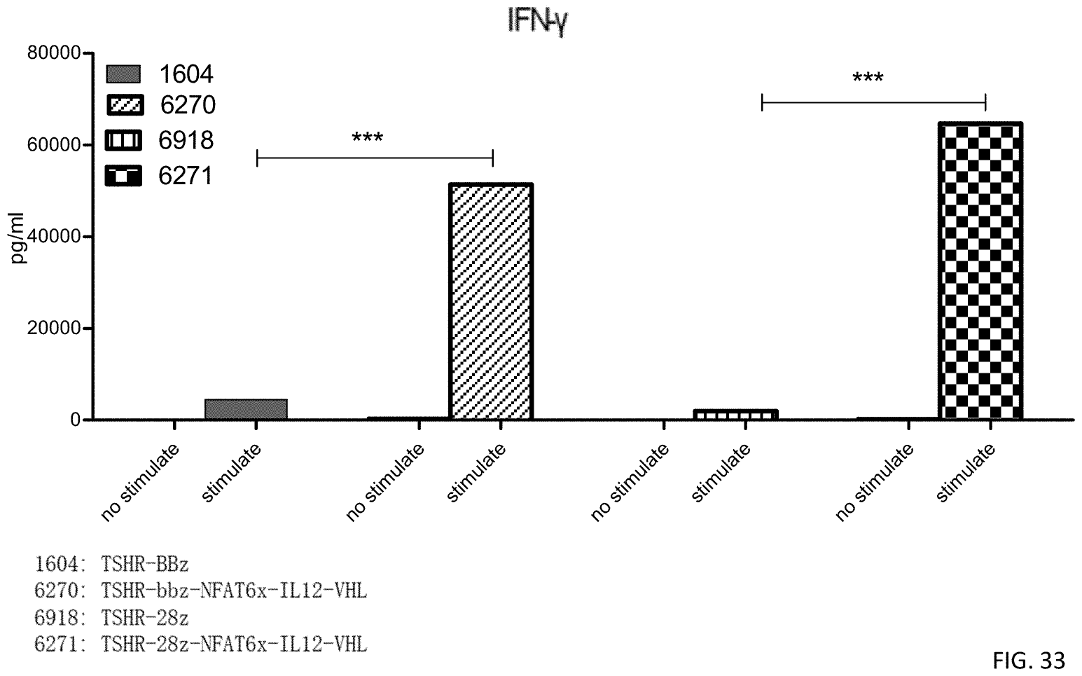

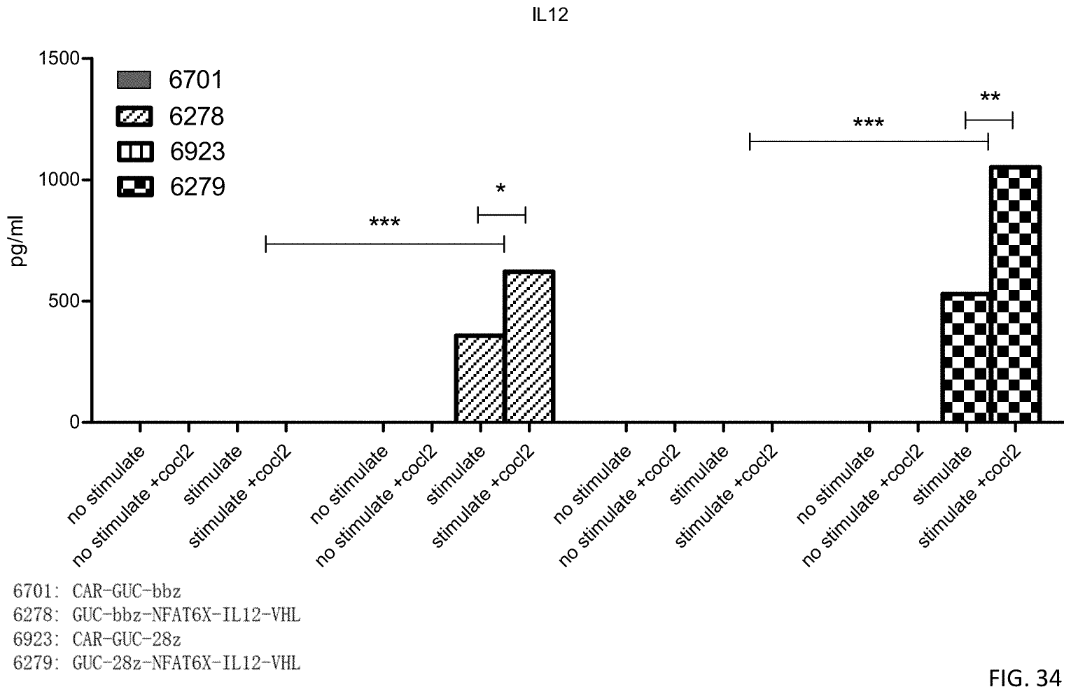

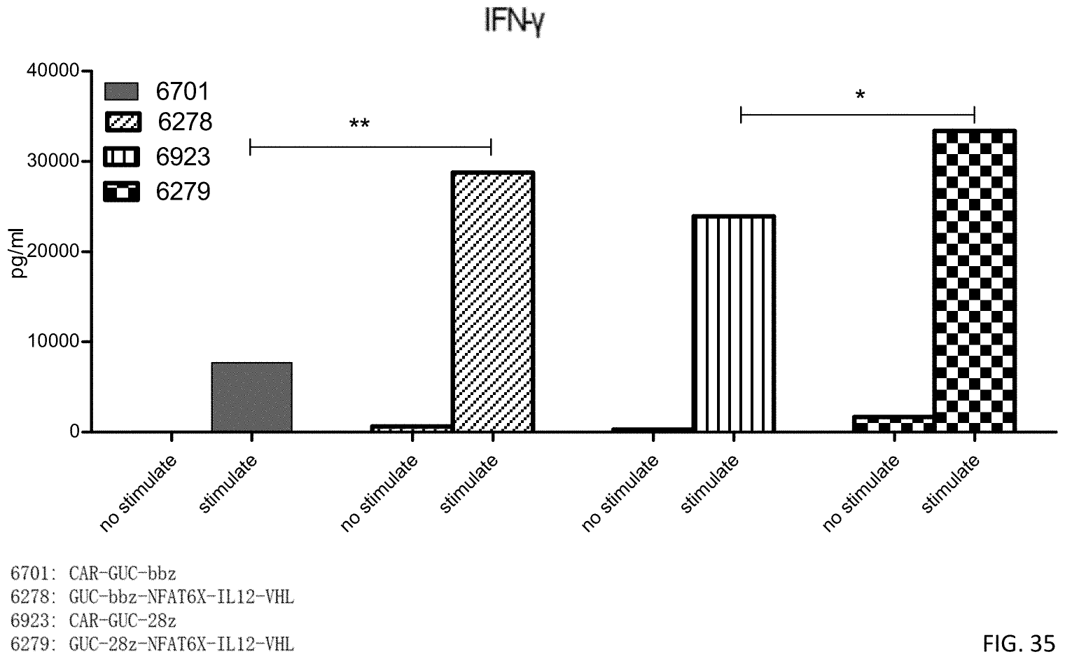

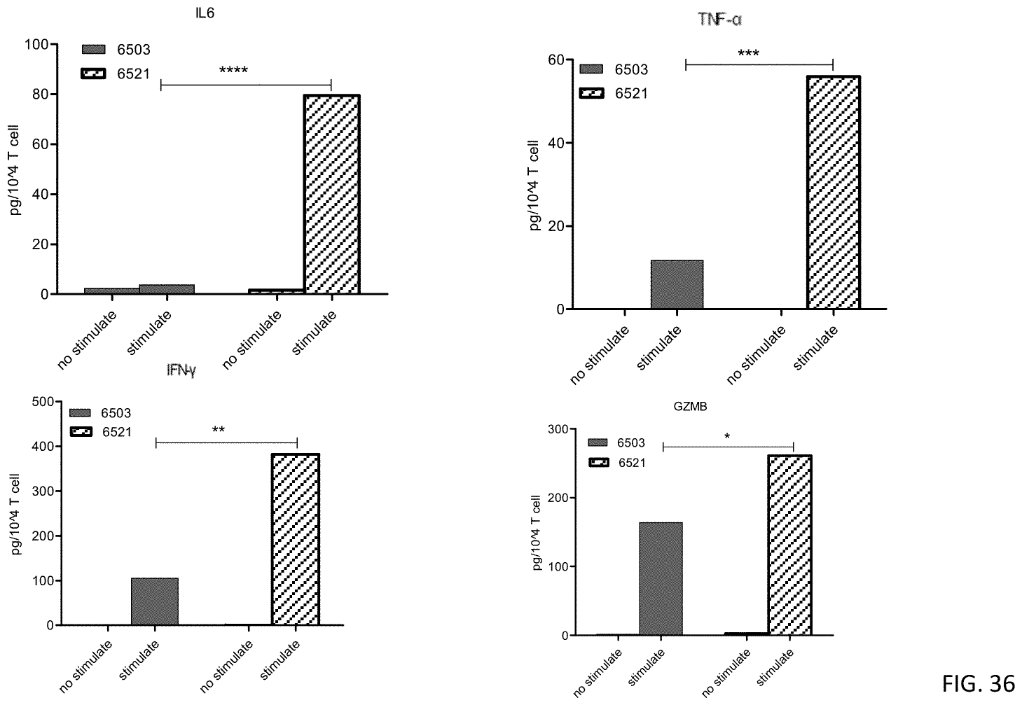

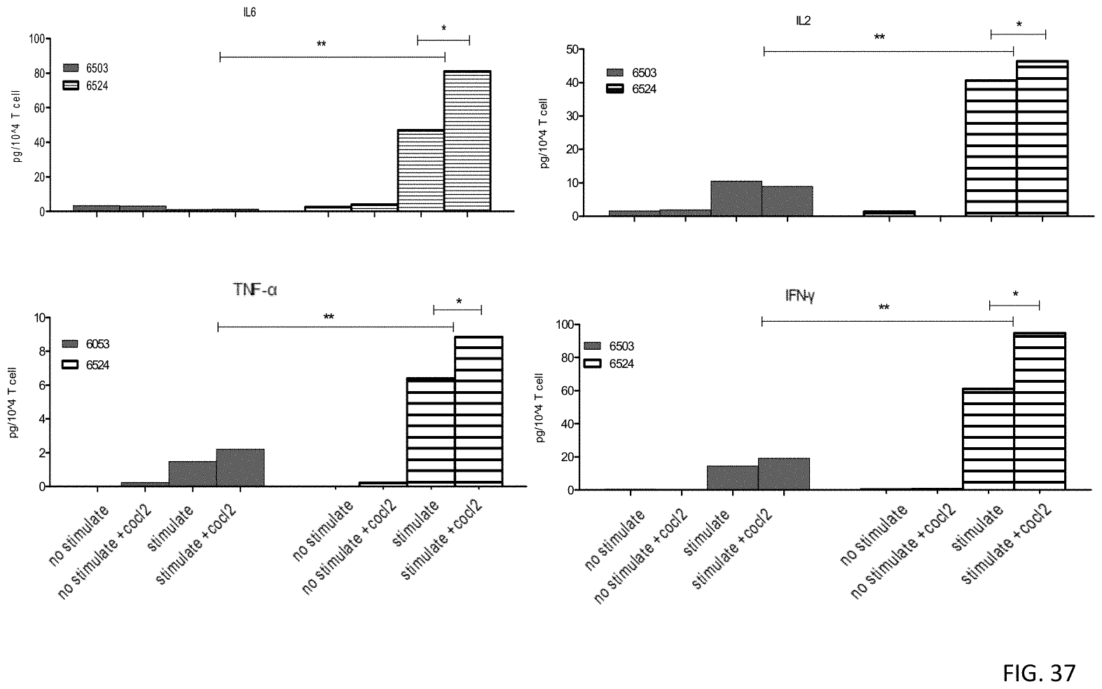

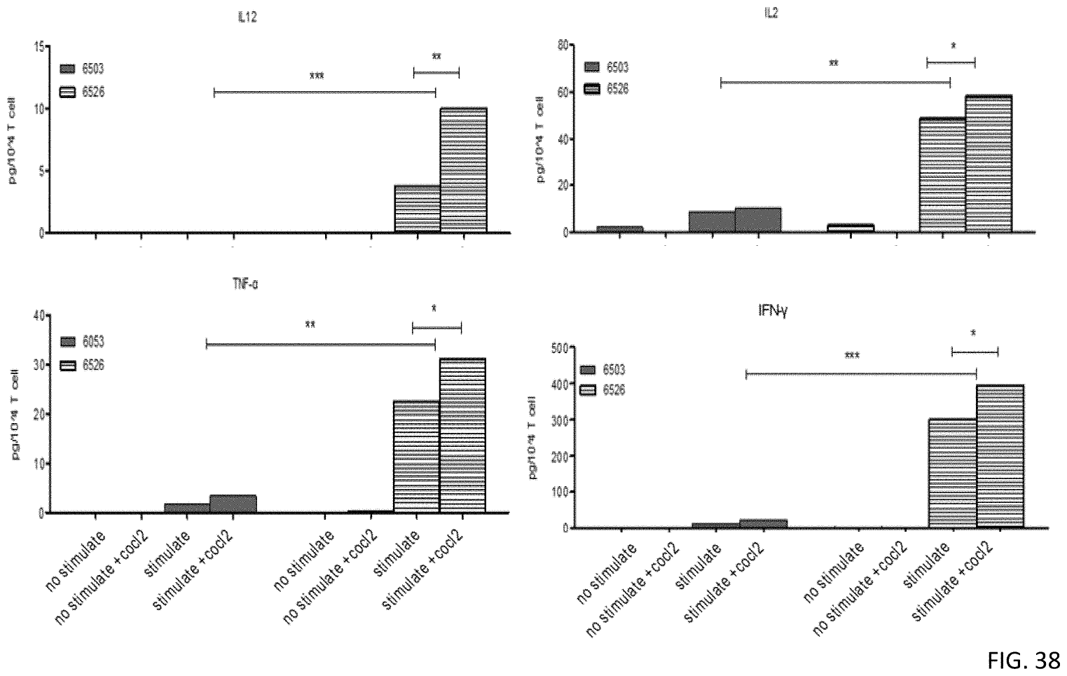

FIG. 30 shows the results of an anaerobic assay. Protocols of manufacture of CAR T cells and cell culturing were similar for those in vitro assays described herein. In the anaerobic system simulated by CoCl₂, it can be seen from FIG. 30 that the release of IL-12 is regulated by oxygen. With the prolongation of CoCl2, the release of IL-12 gradually increases, and IL-12 decreases significantly after 48 hours of withdrawal of CoCl2. FIG. 31 shows cytokine releases in response to hypoxia in TSHR-CART system. As shown, IL-12 was only expressed when 6270 and 6271 were activated and was regulated by the anaerobic switch VHL, and the release of IL12 was significantly increased under anaerobic conditions. FIGS. 32 and 33 show cytokine release in response to induction of IL-12 expression in CAR T cells. The expression of IL12 effectively up-regulated the expression of IFNγ and TNF-α, indicating that the system can further increase the release of IFNγ/TNF-α and enhance the function of CAR T cells by regulating the expression of IL-12. FIG. 34 shows cytokine release in response to hypoxia in GUCY2C-CAR T system. FIG. 35 shows IFNγ release in response to induction of IL-12 expression in CAR T cells. FIG. 36 shows IL-6 is induced by T cell activation, and T cell inactivation does not express IL-6; and induction of IL-6 expression can up-regulate the release of T cell killing function-related factors such as TNF-α, IFNγ, and GZMB. FIGS. 37 and 38 show cytokine release in response to hypoxia in ACPP-CAR T system. Further data show that the expression of IL-6, IFNγ, and/or IL-12 effectively up-regulated the expression of other inflammatory factors such as GZMB and TNF-α, thereby enhancing the function of CAR T cells.

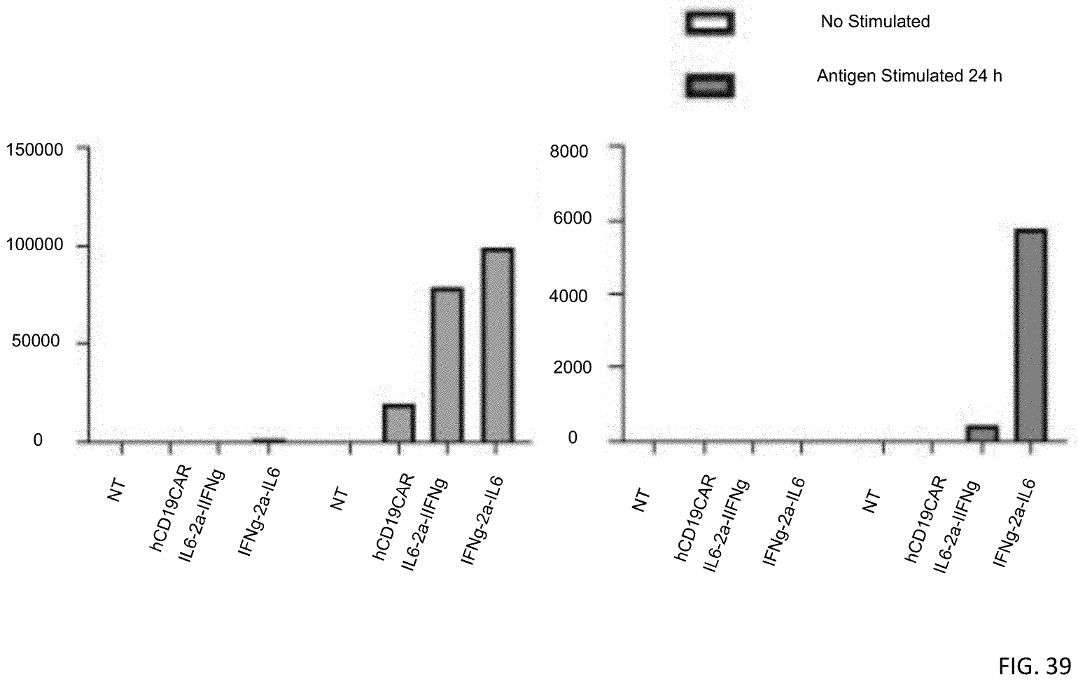

FIG. 39 is cytokine release assay showing that IL-6 and IFNγ released by various types of CAR T cells under different conditions after the CAR T cells were cultured with or without antigen for 24 hours. The results show that CAR T cells expressing IFNγ-2A-IL-6 released more IL-6 and IFNγ in response to antigen activation as compared to (1) CAR T cells not expressing IL-6 and IFNγ and (2) CAR T cells expressing IL-6-2A-IFNγ. The data may indicate that CAR T cells comprising construct IFNγ-2A-IL-6 may have increased anti-tumor effect than CAR T cells comprising construct IL-6-2A-IFNγ.

### In Vivo Cell Expansion and Treatment of Cancer

These clinical studies were designed to assess the safety and efficacy of infusing autologous T cells modified to express TSHR specific CAR/4-1 BB/CD3-ζ into patients. On the first arm of the studies, patients received TSHRCAR T cells only. On the second arm, patients received CART cells that bind 0019 and TSHR and express IL-6 and IFNγ, and/or IL-12. Autologous T cells modified to express TSHR specific CAR/4-1BB/CD3-ζ (TSHRCAR), 0019 specific CAR/4-1BB/CD3-ζ (CD19CAR), and IL-6 and IFNγ, and/or IL-12 were infused into patients. T cells from the patients were obtained, modified, and infused to the patients. T cell response of patients from the first and second arms were measured and compared using the following protocols, which were approved by the hospitals where the trials were conducted. All patients were provided with written informed consent. In the discussion below, the following abbreviations are used: SD: stable disease; PD: progressive disease; PR: partial remission; CR: complete remission; and NR, no response.

TABLE 10

| | Patient | | | | Safety or | |
|---|---|---|---|---|---|---|
| | ID | Target | Infusion | Vectors mixed | Adverse | |
| Cancer | (PID) | Marker | CART/kg | with T cells | reaction (AR) | Efficacy |
| Thyroid | 004 | TSHR | 1.1 × 10⁶ | Vector encoding | No apparent | NR |
| cancer | | | | TSHR-CAR | AR | |
| Thyroid | 005 | TSHR | 2.08 × 10⁹ | Vectors encoding | No apparent | PR (day |
| cancer | | | | TSHR-CAR & | AR (See below) | 90) |
| | | | | vectors encoding | | |
| | | | | CD19-CAR | | |
| Thyroid | 006 | TSHR | 1.36 × 10⁹ | Vectors encoding | No apparent | PR (day |
| cancer | | | | TSHR-CAR & | AR (See below) | 30) |
| | | | | vectors encoding | | |
| | | | | CD19-CAR-IL-6-2A- | | |
| | | | | IFNγ & vectors | | |
| | | | | encoding CD19- | | |
| | | | | CAR-IL-12 | | |
| Colorectal | 007 | GUCY2C | 3.78 × 10⁸ | Vectors encoding | No apparent | PR (day |
| cancer | | | | TSHR-CAR & | AR (See below) | 30) |
| | | | | vectors encoding | | |
| | | | | CD19-CAR-IL-6-2A- | | |
| | | | | IFNγ | | |

PBMCs were obtained from patients. Various lentiviral vectors were generated and then transfected into the T cells from the PBMCs, which were further cultured for several days before the co-cultivation assay. For Patients 004-007, information is provided in Table 11 and the descriptions below. Techniques related to cell cultures and cytotoxic T lymphocyte assay may be found in “Control of large, established tumor xenografts with genetically retargeted human T cells containing CD28 and CD137 domains,” PNAS, Mar. 3, 2009, vol. 106 no. 9, 3360-3365, which is incorporated herein by reference in its entirety.

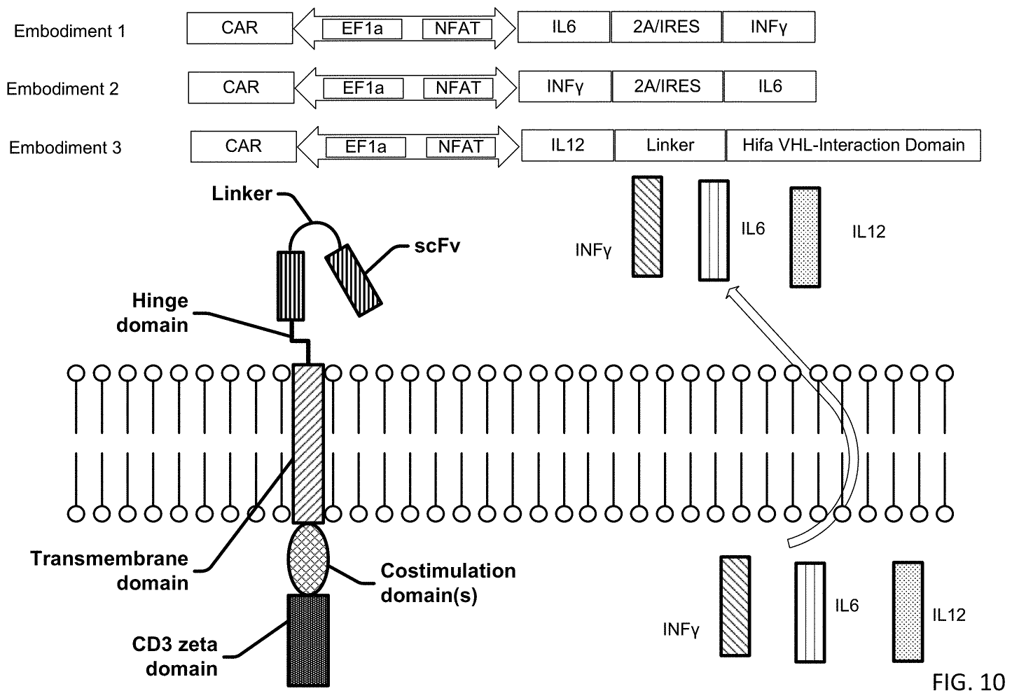

TABLE 11

| | | Infusion | |
|---|---|---|---|
| PID | Vectors and MOI | Methods | Pre-treatment |
| 004 | Vector 1: TSHR-CAR (CAR: SEQ ID NO: 279, | Fresh | FC regimen at −5 to |
| | scFv of the CAR: SEQ ID NO: 8): 19:1 (MOI) | cells | −3 days |
| | | | (cyclophosphamide |
| | | | 500 mg/m2, |
| | | | fludarabine 30 mg/m2) |
| 005 | Vector 1: TSHR-CAR (CAR: SEQ ID NO: 279, | Fresh | FC regimen at −5 to |
| | scFv of the CAR: SEQ ID NO: 8): 19:1(MOI); and | cells | −3 days |
| | Vector 2: hCD19-CAR-NATF-IL6-2A-IFNγ (Vector | | (cyclophosphamide |
| | SEQ ID NO: 532, scFv of CD19 CAR: SEQ ID 5, | | 500 mg/m2, |
| | 6xNFAT: SEQ ID: 469, aa of IL6: SEQ ID NO: 287, | | fludarabine 30 mg/m2) |
| | 2A is SEQ ID NO: 327, and aa of IFN-γ: SEQ ID | | |
| | NO: 328 (See the construct of Embodiment 1 of | | |
| | FIG. 10)): 5:1(MOI) | | |
| 006 | Vector 1: TSHR-CAR (CAR: SEQ ID NO: 279, | Fresh | FC regimen at −5 to |
| | scFvofthe CAR: SEQ ID NO: 8): 50:1(See MOI in | cells | −3 days |
| | Table 12); Vector 2: hCD19-CAR-NATF-IL6-2A- | | (cyclophosphamide |
| | IFNγ (Vector SEQ ID NO: 532, scFv of CD19 | | 500 mg/m2, |
| | CAR: SEQ ID 5, 6xNFAT: SEQ ID: 469, aa of IL6: | | fludarabine 30 mg/m2) |
| | SEQ ID NO: 287, 2A is SEQ ID NO: 327, and aa | | |
| | of IFN-γ: SEQ ID NO: 328 (See the construct in | | |
| | Embodiment 1 of FIG. 10)): 10:1 (See MOI in Table | | |
| | 12); and Vector 3: hCD19-CAR-NATF-IL12-VHL | | |
| | (Vector SEQ ID NO: 533, scFv of CD19 CAR: | | |
| | SEQ ID 5, 6xNFAT: SEQ ID: 469, aa of IL12: SEQ | | |
| | ID NO: 450, VHL: SEQ ID NO: 457 (See the | | |
| | construct of Embodiment 3 of FIG. 10): 1:1 (See | | |
| | MOI in Table 12). | | |
| 007 | Vector 4: GUCY2C-CAR (CAR: SEQ ID NO: 535, | Fresh | FC regimen at −5 to |
| | scFv of the CAR: SEQ ID NO: 11): 50:1 (MOI); and | cells | −3 days |
| | Vector 2: hCD19-CAR-NATF-IL6-2A-IFNγ (Vector | | (cyclophosphamide |
| | SEQ ID NO: 532, scFv of CD19 CAR: SEQ ID 5, | | 500 mg/m2, |
| | 6xNFAT: SEQ ID: 469, aa of IL6: SEQ ID NO: 287, | | fludarabine 30 mg/m2) |
| | 2A is SEQ ID NO: 327, and aa of IFN-γ: SEQ ID | | |
| | NO: 328 (See the construct of Embodiment 1 of | | |
| | FIG. 10)): 10:1(MOI) | | |

PBMCs were cultured using TEXMACS culture containing IL-2. CD4 and CD8 magnetic beads were used to sort and select T cells in the PBMCs. The appropriate starting culture amount was selected, and TransAct activator was used to activate T cells (CD3+ cells). MACS® GMP T Cell TransAct™ includes a colloidal polymeric nano matrix covalently attached to humanized recombinant agonists against human 003 and 0028. Due to the nano matrix MACS, GMP T Cell TransAct can be sterile filtered, and excess reagent can be removed by centrifugation and following conventional supernatant replacement or simply by medium wash. This reagent is suitable for use in automated culture systems, such as the CliniMACS Prodigy® Instrument. The number of corresponding vectors and the volume of the vector were calculated according to the required vector MOI (See Table 11). For Patient 004, lentiviral vectors containing Vector 1 were mixed, for 24 hours, with the T cells, which were further washed and cultured for 8 days before being transported to the hospital. For Patients 005 and 007, lentiviral vectors containing Vector 1 and Vector 2 were mixed with the T cells for 24 hours. The T cells were further washed and cultured for 8 days before being transported to the hospital. For Patient 006, the T cells were divided into four groups, and each group of T cells were mixed with one or more lentiviral vectors for 24 hours (see Table 12), and these T cells were washed and cultured for 8 days. These four groups of transfected T cells were mixed and then transported to the hospital.

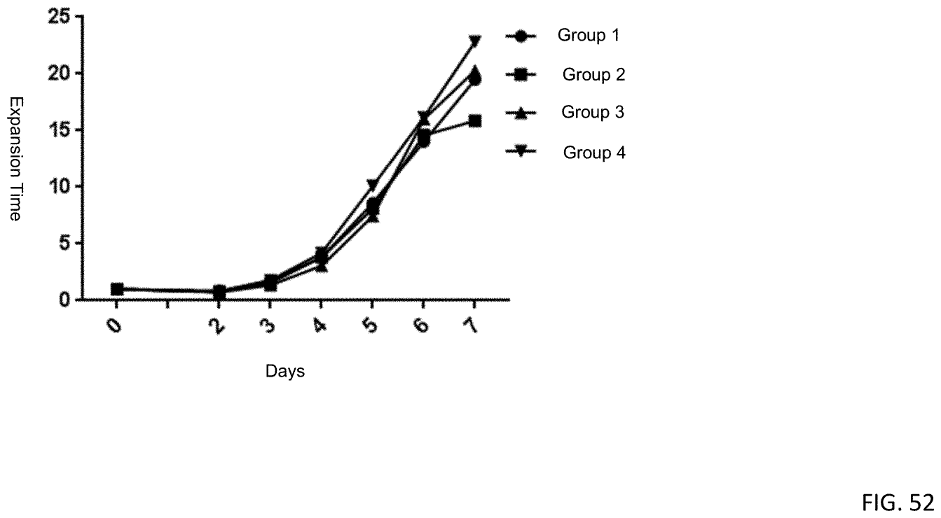

Various assays were performed to confirm the efficacy of these CAR T cells (e.g., a binding assay using flow cytometry and a killing assay using culturing assay with cells expressing TSHR/CD19), and quality assurance procedures were followed to ensure the safety of the administration of the CAR T cells to the patient. Independent primers of TSHR, IL-6 and IFN-γ, and/or IL-12 were used to detect the percentage of total vectors in CAR T cells by quantitative PCR (qPCR). The percentage was obtained by relative quantification using three vectors. The number of cycles at the threshold is used to determine the starting amount of the sample. For example, for Patient 006, the starting amount of IL-12 was defined as 1, and the relative quantification of IL-6, IFN-γ, and TSHR was calculated, and then the relative quantification of the three vectors was used as the denominator to calculate the percentage of each vector in the total amount. Further, the 4-1 BB universal primer was used to detect the CAR copy number (i.e., the total CAR copy number) in the CAR T cells by qPCR. In addition, when Patient 006's CAR T cells were expanded in vitro to day 6, samples of the T cells were taken. The sample T cells were labeled with antibodies against hCD3, and T cells having CD19CAR and/or IL-6/IFN-γ vectors were labeled with antibodies against hCD19 scFv. Flow cytometry analysis was performed, and the results were used to calculated and obtain the ratio of T cells expressing CD19 CAR in total transfected T cells. These results are shown in Tables 12 and 13 as well as FIG. 52, which shows the expansion of T cells in each group.

TABLE 12

| | | | Day 6 | | Total CAR | Ratio to Total | | |
|---|---|---|---|---|---|---|---|---|
| | | | hCAR+/ | CD19 | copy number | IL-6/ | | |
| Group | Vector | MOI | CD3+ | scFv+/CD3+ | per ugDNA | IFN-γ | IL12 | TSHR |
| 1 | 1 | 50 | 42.64% | 12.37% | 112238.97 | 8.27% | 0.21% | 91.52% |
| | 2 | 10 | | | | | | |
| | 3 | 1 | | | | | | |
| 2 | 1 | 50 | 59.28% | 33.32% | 118925.1 | 17.77% | 0.34% | 81.89% |
| | 2 | 10 | | | | | | |
| | 3 | 1 | | | | | | |
| 3 | 3 | 20 | 19.52% | 18.16% | 19408.75 | 0.00% | 100.00% | 0.00% |
| 4 | 3 | 20 | 26.70% | 26.59% | 17874.89 | 0.00% | 100.00% | 0.00% |

TABLE 13

| Expansion times/ | | | | |
|---|---|---|---|---|
| Culturing Days | Group 1 | Group 2 | Group 3 | Group 3 |
| 0 | 1.00 | 1.00 | 1.00 | 1.00 |
| 2 | 0.87 | 0.78 | 0.74 | 0.78 |
| 3 | 1.76 | 1.56 | 1.37 | 1.80 |
| 4 | 3.80 | 3.83 | 3.08 | 4.20 |
| 5 | 8.60 | 8.10 | 7.45 | 10.10 |
| 6 | 14.04 | 14.60 | 16.01 | 16.17 |
| 7 | 19.49 | 15.86 | 20.28 | 22.82 |

For fresh cells, after removing the magnetic beads, the transduced cells were centrifuged or replaced with a solution of 95% compound electrolyte injection and 5% human albumin, loaded into a return bag and transported at 15-25° C. after sealing. Fresh preparations were returned directly. For cryopreserved cells, the transduced cells were transferred to the media including a compound electrolyte injection of 33.75% human albumin, 25% dextran, 40% glucose, and 33.75% DMSO 7.5%. The cell suspension was loaded into a cryopreservation bag, and then the procedure was cooled to −90° C. and transferred to a gas phase liquid nitrogen tank for storage. The reconstitution of the frozen preparations was completed within 30 minutes after resuscitation. PBMCs were obtained from patients by leukapheresis for CAR T cell preparation, and the first day of CAR T infusion was set as day 0.

Patients were given a conditioning treatment for lymphodepletion. Fludarabine- and cyclophosphamide-based conditioning treatment varied according to the tumor burden in the bone marrow (BM) and peripheral blood (PB). CAR T cells were transfused to patients. CAR T cells were transported to the hospital, washed, counted, checked for viability, and then prepared for administration to patients. After administration, the patients were observed closely for at least 2 hours. Cytokine Release Syndrome (CRS) was graded according to a revised grading system (see Lee D W. et al., Blood 2014; 124:188-95). Other toxicities during and after therapy were assessed according to the National Institutes of Health Common Terminology Criteria for Adverse Events Version 4.0 (http://ctep.cancer.gov/). Therapy responses were assessed by flow cytometry and morphological analysis. When possible, patients were assessed for levels of chimeric gene expression. The response type was defined as minimal residual disease (MRD) negative, complete response, complete response with incomplete count recovery, stable disease, and progressive disease.

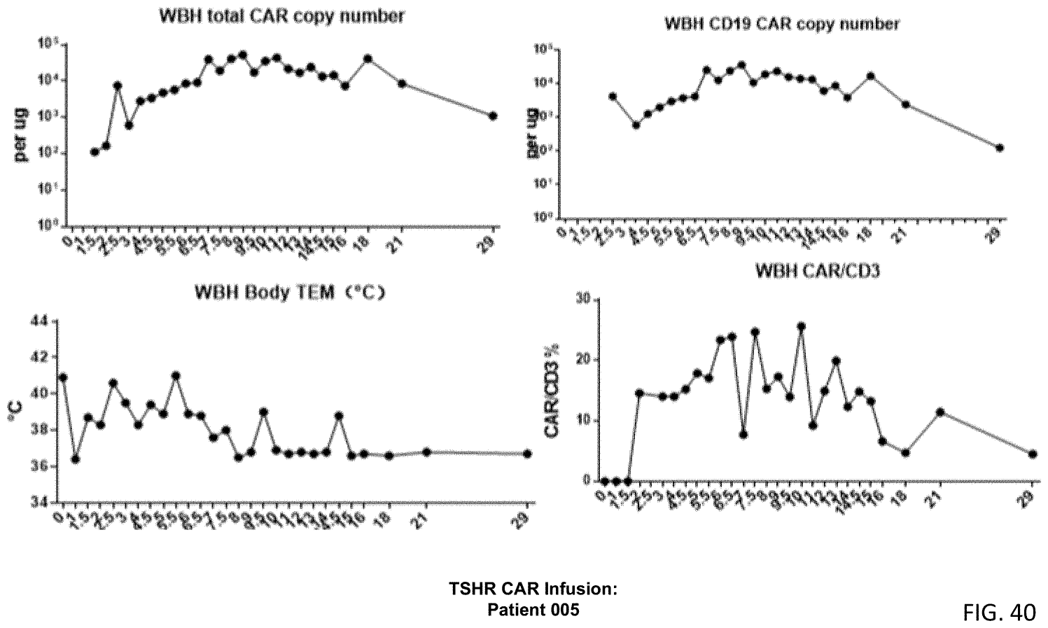

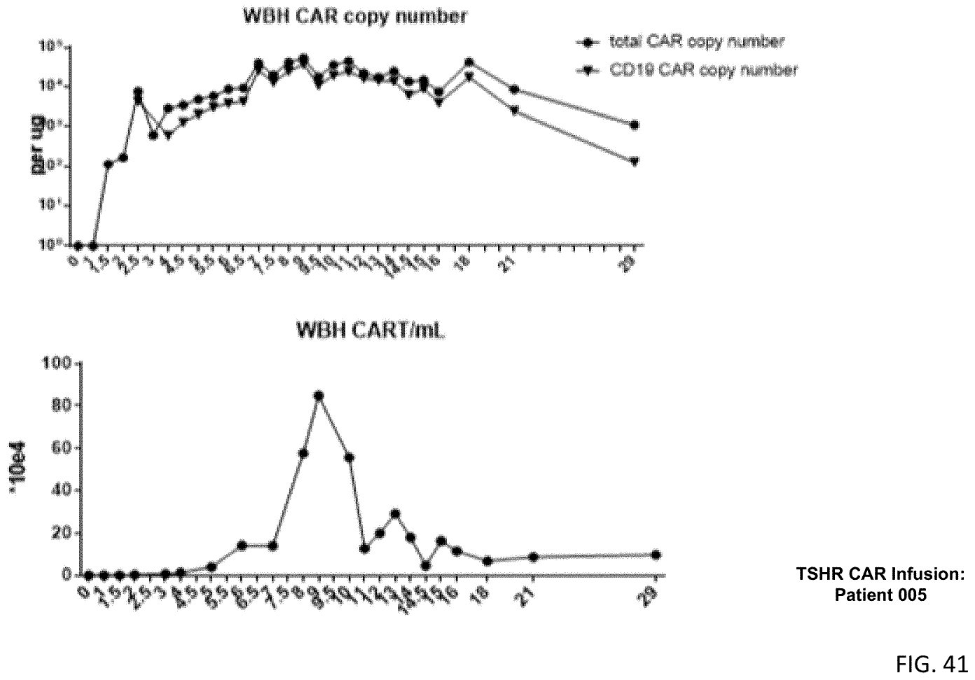

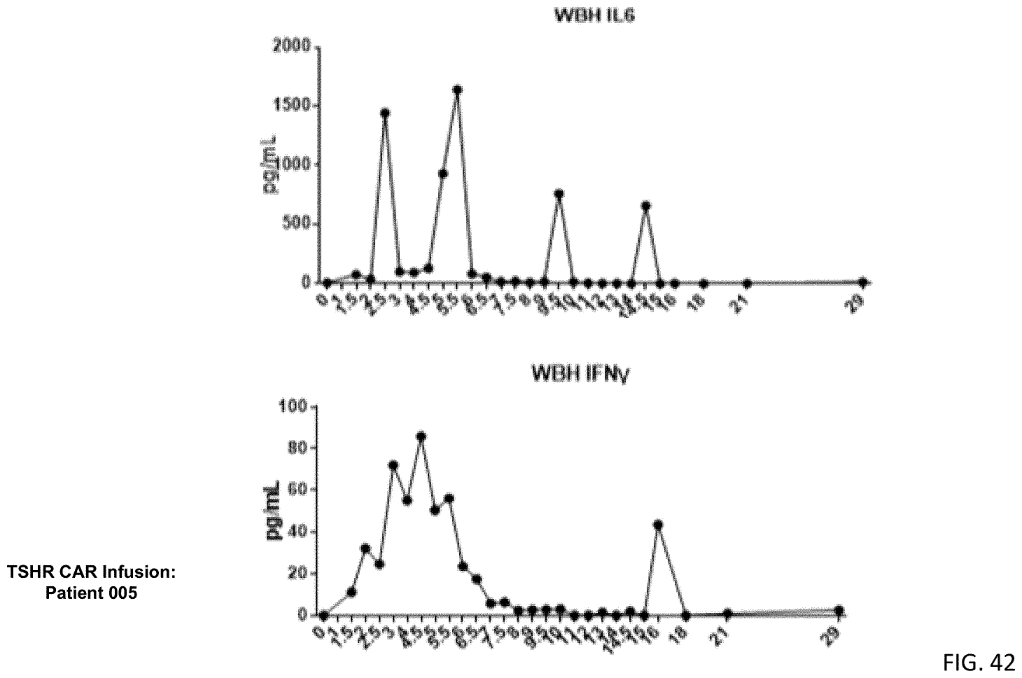

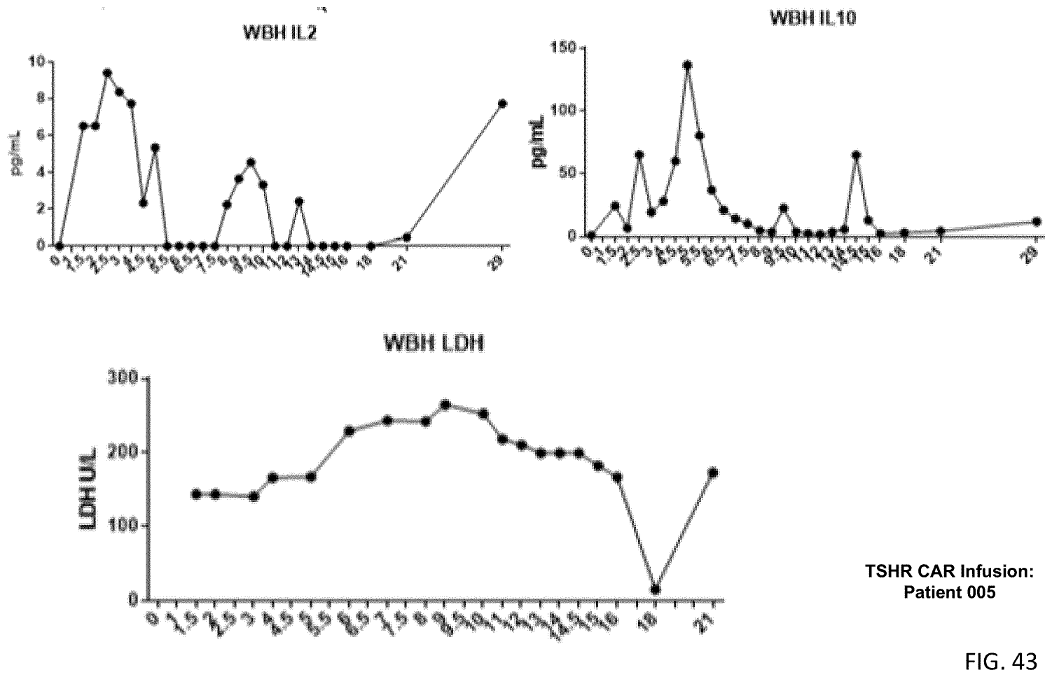

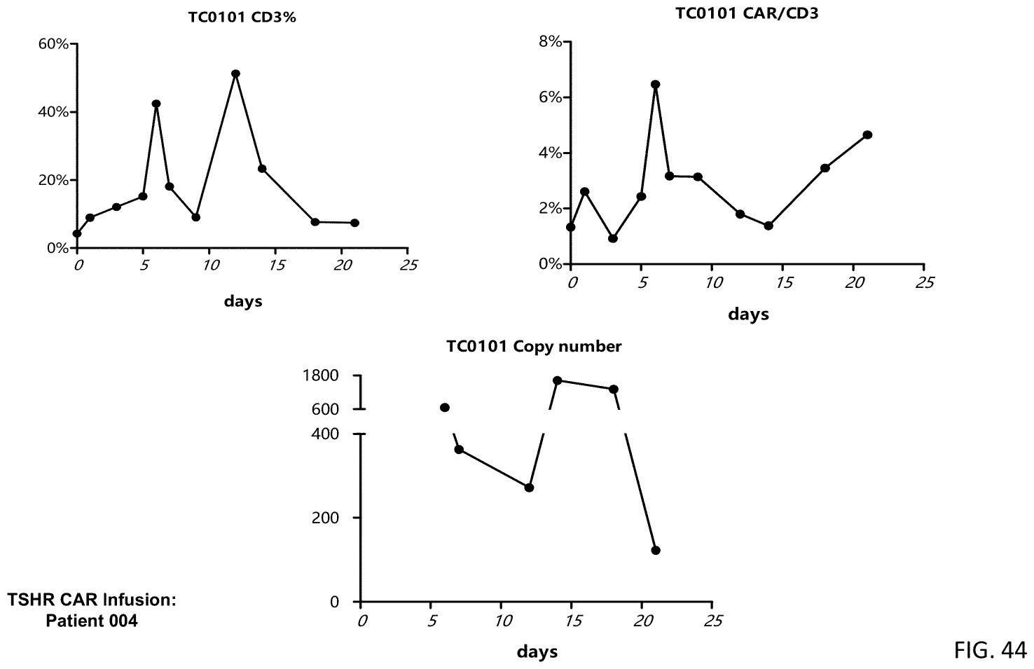

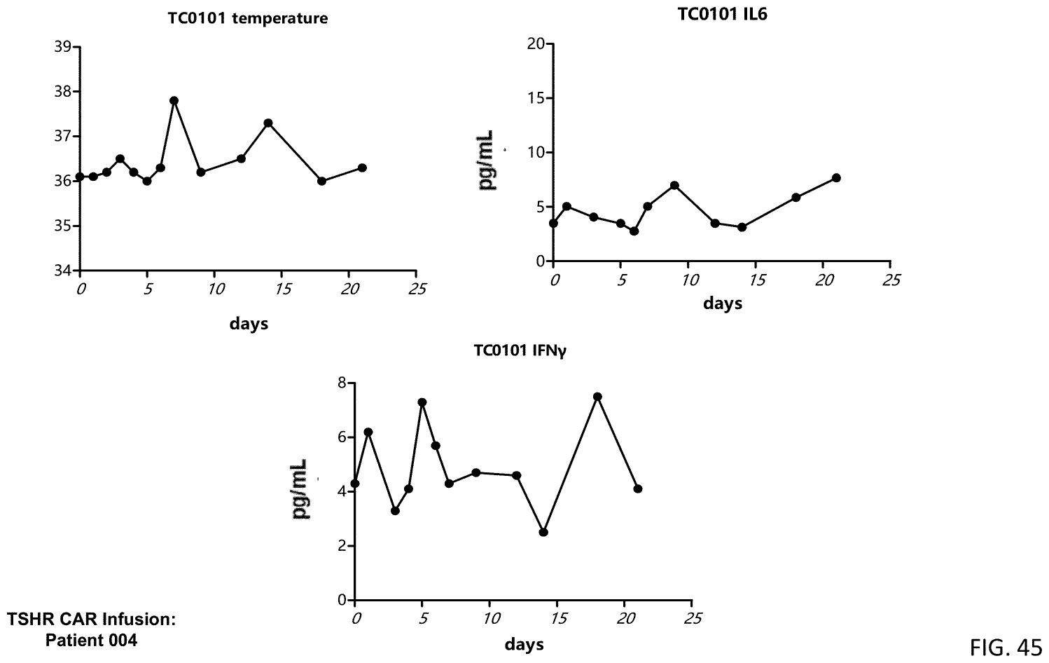

Serial BM and PB samples after CAR T cell infusion were collected in K2EDTA BD vacutainer tubes (BD). The persistence of CD19CAR T cells from fresh PB and BM in patients was determined by FACS. Circulating CAR T cell numbers per μl were calculated on the basis of measured absolute CD3+ T lymphocyte counts. Simultaneously, CAR DNA copies were evaluated as another method of determining CAR T cell expansion and persistence. Genomic DNA was extracted using a QlAamp DNA Blood Mini Kit (Qiagen) from cryopreserved PB and BM. CAR DNA copies were assessed by quantitative real-time PCR as described in the supplementary materials. The levels of cytokines IFN-γ, TNF-α, IL-2, IL-4, IL-6, IL-10, IL-17, etc. in serum and CSF were measured in a multiplex format according to the manufacturer's instructions (see FIGS. 40-47). Genomic DNA from cryopreserved peripheral blood and bone marrow was extracted using a QlAamp DNA Blood Mini Kit (Qiagen). Quantitative PCR (q-PCR) was performed in real-time in triplicates using the ABI 2×TaqMan Universal Master Mix with AmpErase UNG (Applied Biosystems) in a 7500 real-time PCR system (Applied Biosystems). Copy numbers per microgram of genomic DNA were calculated from a standard curve of 10-fold serial dilutions of purified CAR plasmid containing 102-108 copies/p. Amplification of an internal control gene was used for normalization of DNA quantities. Primers/probes specific for CAR19 and other transgenes and an internal control gene were as previously described (see Gokbuget N. et al., Blood 2012; 120:2032-41 and O'Brien S. et al., J Clin Oncol 2013; 31:676-83). CT Scanning and/or PET CT scanning was performed to evaluate CAR T therapy on patients.

For Patient 004, no apparent response (e.g., disappearance or shrink of target lesions) was observed after cell infusion. The patient also did not have any adverse reaction.

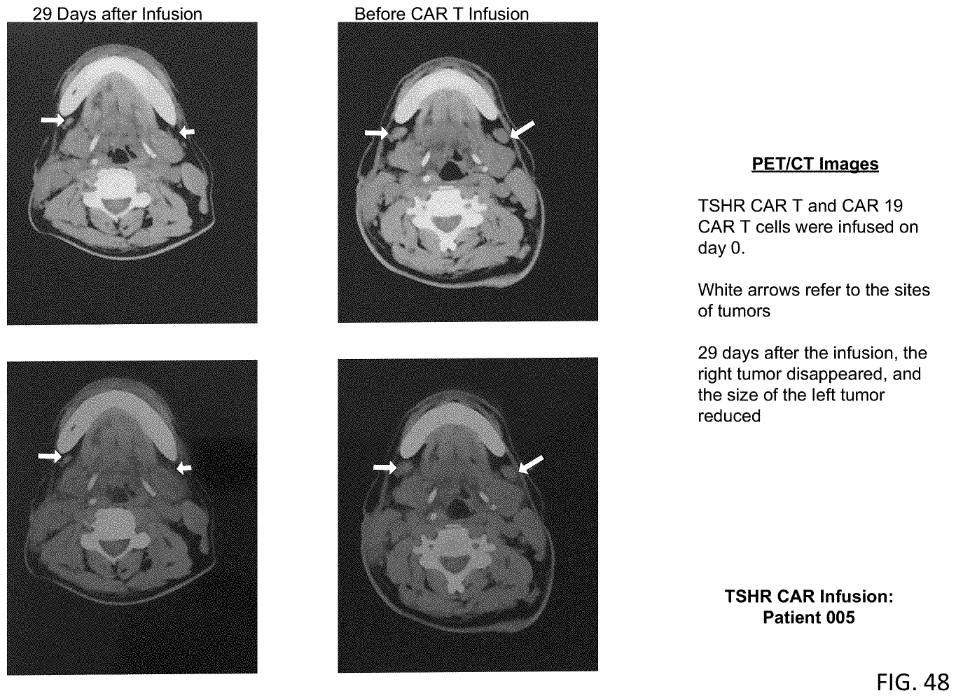

Patient 005 had undergone thyroidectomy. 29 days after the infusion, the right tumor disappeared, and the left tumor was reduced in size. Examples of PET CT scanning images are shown in FIG. 48. Three months after the infusion, the right tumor did not recur, and the left tumor disappeared. The PET CT images showed that there was no observable tumor recurrence, particularly in the surgical area. After the scanning signal was enhanced, no abnormal enhancement signal is observed in the above areas. The double neck II and III areas showed multiple small lymph nodes with a maximum short diameter of no more than 10 mm. No abnormalities were observed in the bilateral submandibular gland morphology based on CT scanning signals. At the same time, the cervical spinal cord morphology and CT signal were normal. It appeared that the patient had achieved at least partial remission (PR). During the treatment, no severe CRS (e.g., no greater than level 2 CRS) was observed in Patient 005. The patient was assessed to have achieved PR.

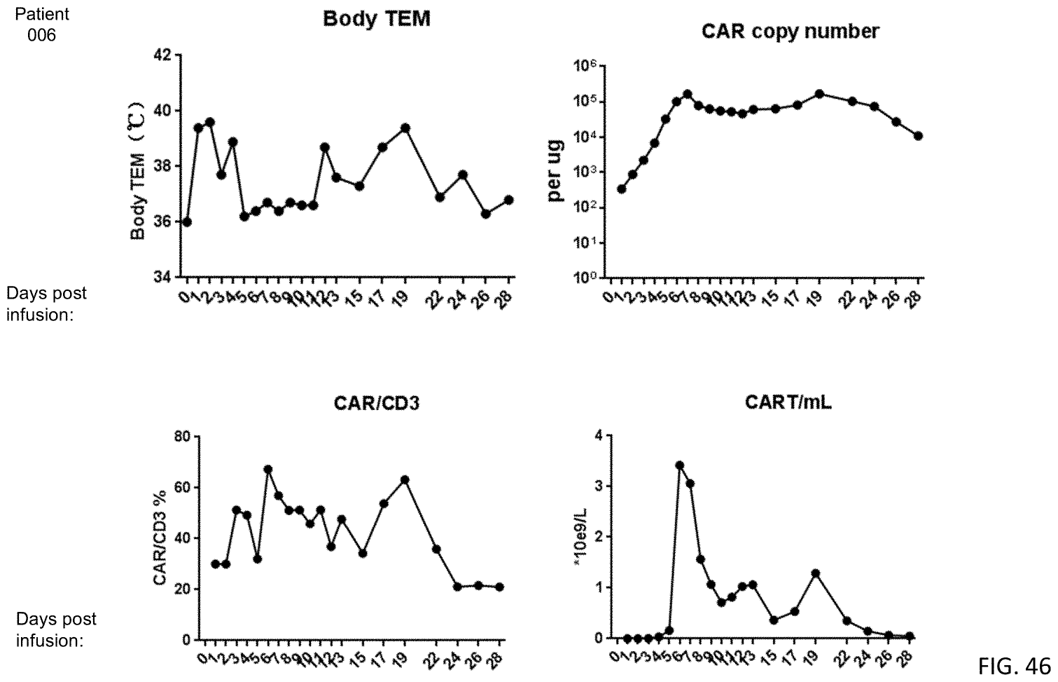

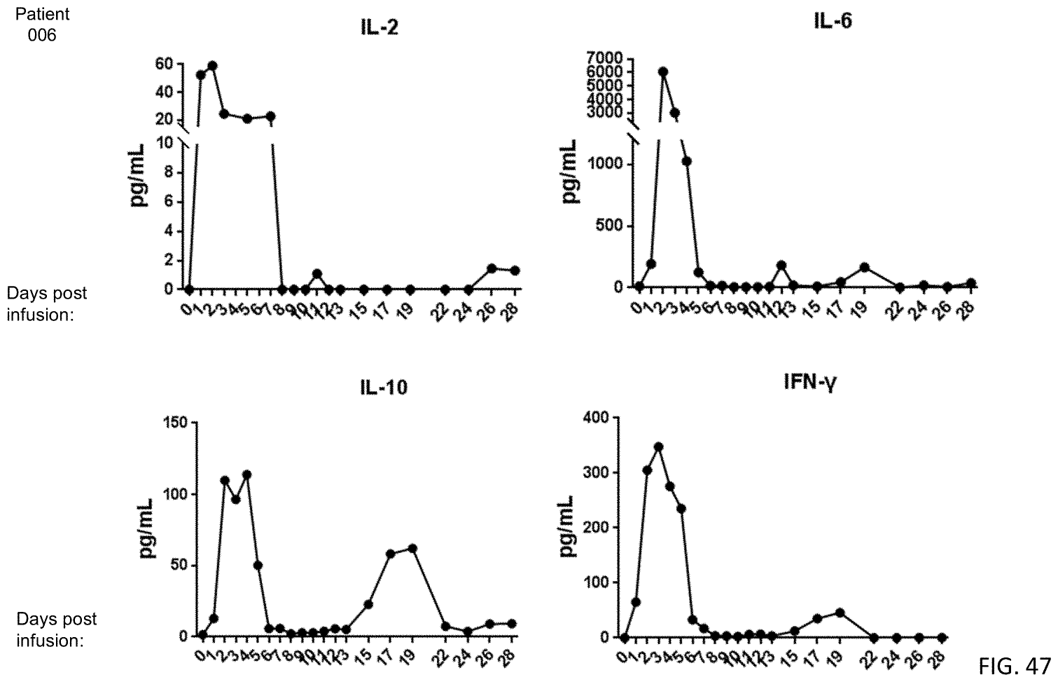

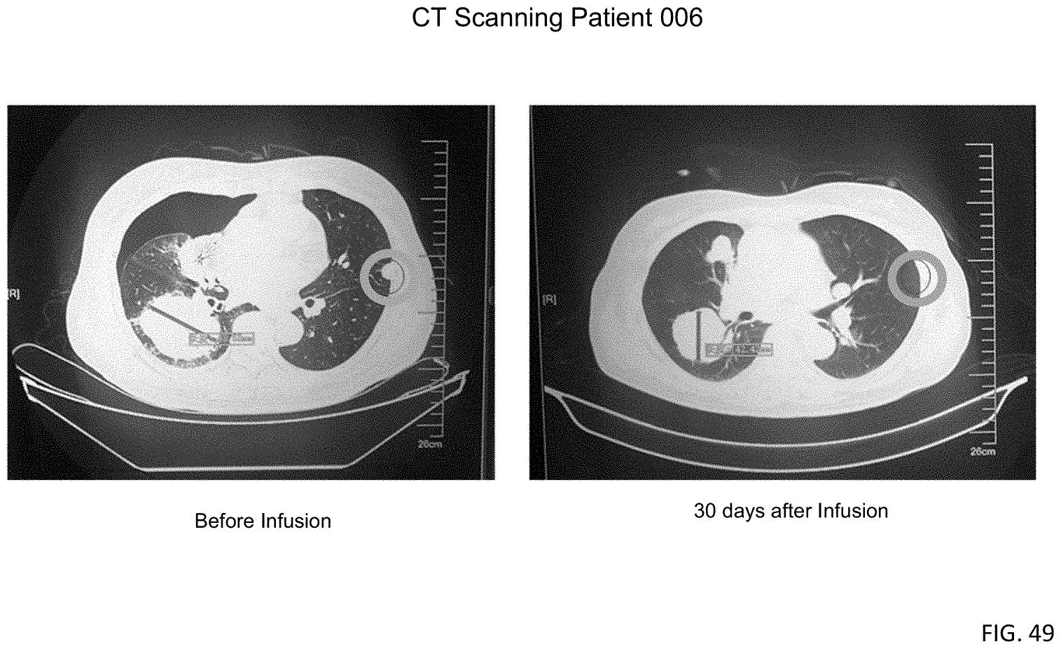

Patient 006 was diagnosed with poorly differentiated follicular papillary carcinoma with neuroendocrine carcinoma in the thyroid gland. Thyroid double lobe resection was performed. The patient was later examined and confirmed to have multiple lung metastases. Multiple enlarged lymph nodes were found in the mediastinum. 30 days after infusion of CAR T cells, CT scanning showed that the small tumors disappeared, and the size of the two major tumor was reduced by more than 70% (see Table 14). FIG. 49 shows that the major tumor shrank, and the small tumor disappeared (see lines as well circles in FIG. 49). During the treatment, no severe CRS (e.g., no greater than level 2 CRS) was observed in Patient 006 (see FIGS. 46 and 47). The patient was assessed to have achieved PR.

TABLE 14

| | Before | Estimated | | Estimated | | Estimated | |
|---|---|---|---|---|---|---|---|
| | Infusion | Volume | Day 4 | Volume | Day 30 | Volume | Reduced |
| Major tumor | (mm) | (mm³) | (mm) | (mm³) | (mm) | (mm³) | Volume |
| Right lower | 68 × 60 | 128112.00 | 70 × 61 | 136312.63 | 42 × 39 | 33431.58 | 73.90% |
| lobe | | | | | | | |
| Mediastinum | 25 | 65416.67 | 25 | 65416.67 | 15 | 14130.00 | 78.40% |
| and double | | | | | | | |
| hilar | | | | | | | |

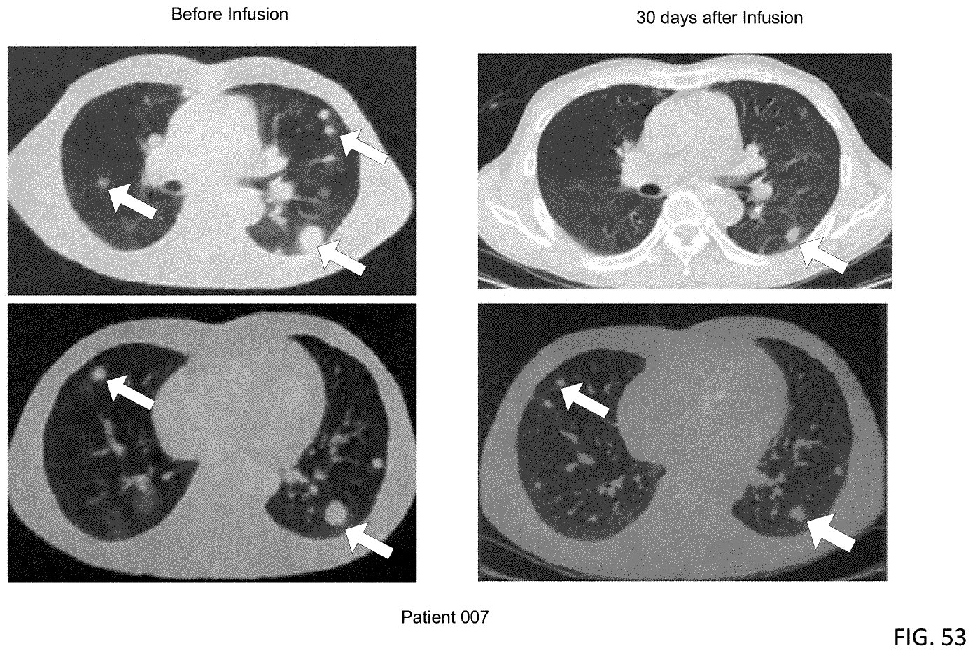

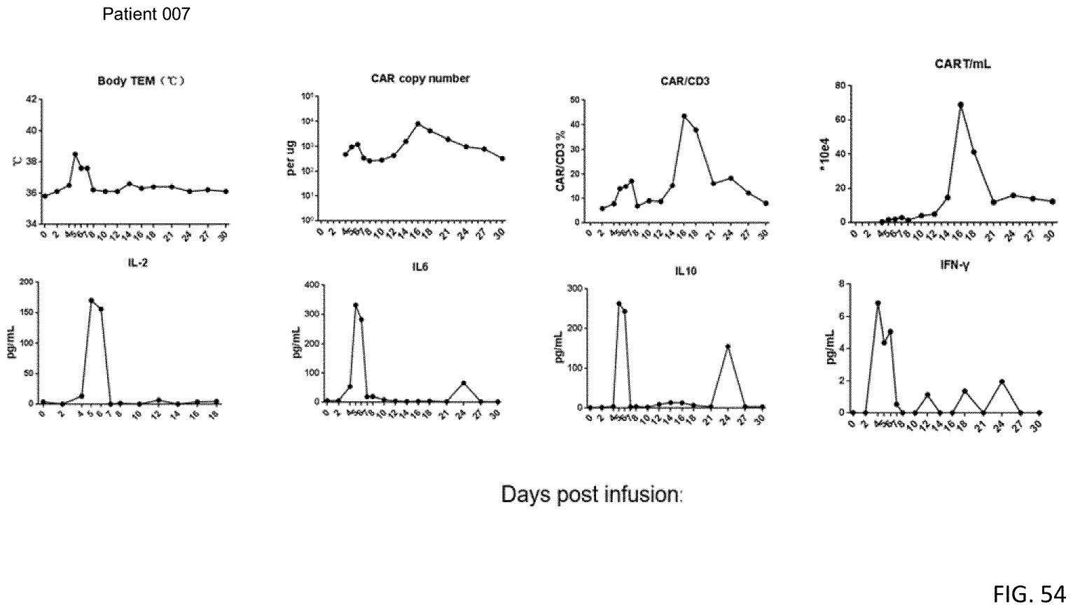

Patient 007 was diagnosed with colorectal cancer and went through 8 cycles of chemotherapy as well as other treatments such as surgery before CAR T cell infusion. One month after the infusion, PET-CT scanning results show most of the target lesions were significantly reduced by more than 50%, and the comprehensive calculation of tumor reduction was 44.7%. The patient was assessed to have achieved PR (see arrows in FIG. 53).

These results demonstrate that, as compared to the infusion of CAR T cells targeting TSHR alone, T cells expressing CD19CAR, TSHRCAR, and IL6/IFNγ and IL-12 enhanced inhibition on the growth of thyroid cancer. Further, T cells expressing CD19CAR and TSHRCAR expanded more and released more cytokines (e.g., IL-6, IL-12 or IFN-γ) as compared to infusion of CAR T cells targeting TSHR alone. Further, CAR T cells expressing IFNγ and IL-6 effectively inhibited the growth of tumor cells, which has not been shown in conventional CAR T technology. Combining the results of the Examples above and the result of this Example seems to indicate that reduction of M2 macrophage caused by T cells expressing IFN-γ may attack tumor microenvironment and thus contribute to the inhibition of the growth of tumor cells.

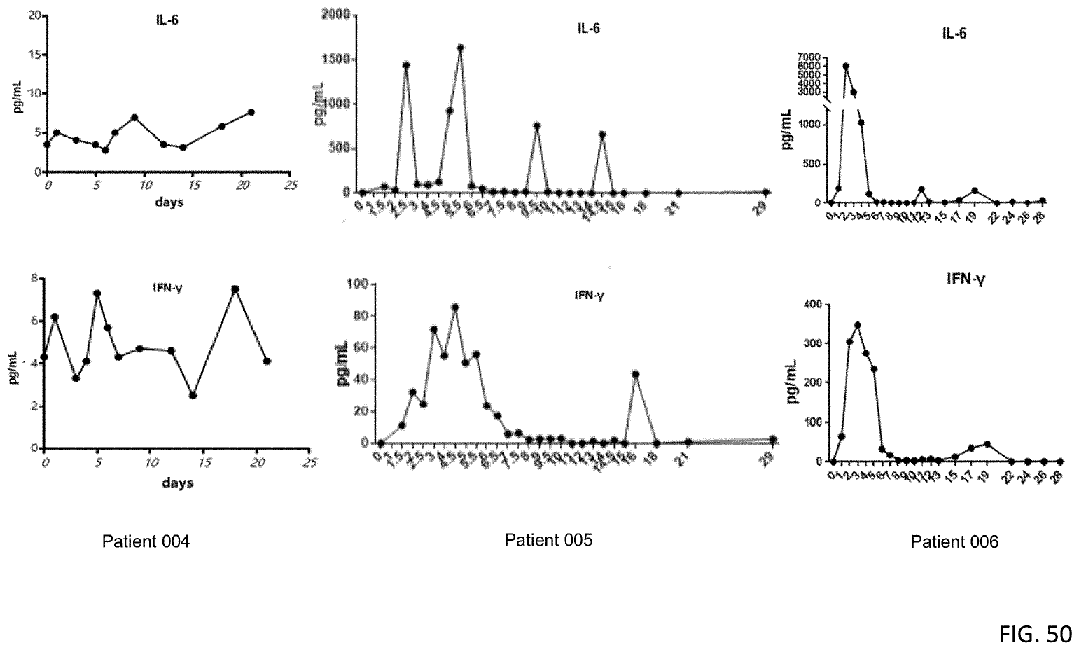

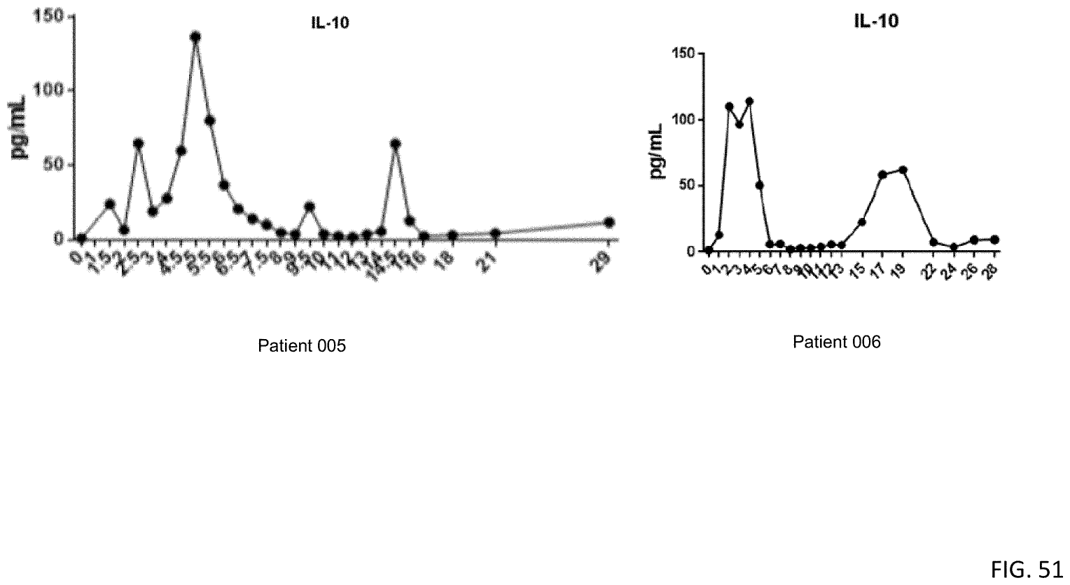

FIGS. 50 and 51 show comparisons of cytokine releases among Patients 004, 005, and 006. As shown in FIG. 50, the comparisons show that CAR T cells in Patients 005 and 006 caused higher levels of IL-6 and IFNγ releases than in Patient 004. Also, CAR T cells in Patient 006 caused higher levels of IL-6 and IFNγ releases than those of Patient 005. As indicated in FIGS. 50 and 51, inducible IL-12 may enhance anti-tumor activities by increasing certain cytokine releases (e.g., IL-6 and IFNγ) but not for those inhibiting anti-tumor activities (e.g., IL-10).

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A composition comprising:
a vector comprising a polynucleotide encoding a chimeric antigen receptor (CAR) binding a solid tumor antigen;
a vector comprising a polynucleotide encoding CAR binding a cell surface molecule of a B cell and a polynucleotide encoding IL-6;
a vector comprising a polynucleotide encoding CAR binding a cell surface molecule of a B cell and a polynucleotide encoding IL-12; and
a vector comprising a polynucleotide encoding CAR binding a cell surface molecule of a B cell and a polynucleotide encoding IFNγ.

2. The composition of claim 1, wherein the cell surface molecule of the B cell is CD19, CD22, CD20, BCMA, CD5, CD7, CD2, CD16, CD56, CD30, CD14, CD68, CD11b, CD18, CD169, CD1c, CD33, CD38, CD138, or CD13.

3. The composition of claim 1, wherein the cell surface molecule of the B cell is CD19, CD20, CD22, or BCMA.

4. The composition of claim 1, wherein the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, CLDN18.2, or EGFR.

5. The composition of claim 1, wherein the CAR comprises an antigen binding domain, a transmembrane domain, a co-stimulatory domain, and a CD3 zeta domain.

6. A method of preparing a mixed population of cells comprising contacting a population of cells with the composition of claim 1, thereby obtaining a mixed population of cells.

7. The method of claim 6, wherein the cells comprise NK cells and/or T cells.

8. The method of claim 6, wherein the cells comprise NK cells or T cells.

9. A composition comprising a vector comprising a polynucleotide encoding a CAR binding a solid tumor antigen and a vector comprising a polynucleotide encoding CAR binding a cell surface molecule of a white blood cell (WBC), and a polynucleotide encoding a cytokine.

10. The composition of claim 9, wherein the white blood cell is a B cell.

11. The composition of claim 9, wherein the cell surface molecule is CD19, CD22, CD20, BCMA, CD5, CD7, CD2, CD16, CD56, CD30, CD14, CD68, CD11b, CD18, CD169, CD1c, CD33, CD38, CD138, or CD13.

12. The composition of claim 9, wherein the cell surface molecule is CD19, CD20, CD22, or BCMA.

13. The composition of claim 9, wherein the solid tumor antigen is tMUC1, PRLR, CLCA1, MUC12, GUCY2C, GPR35, CR1L, MUC17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, CLDN18.2, or EGFR.

14. The composition of claim 9, wherein the CAR comprises an antigen binding domain, a transmembrane domain, a co-stimulatory domain, and a CD3 zeta domain.

15. The composition of claim 9, wherein the cytokine is IL-12, IL-6, or IFNγ.

16. The composition of claim 9, wherein the cytokine is IL-12.

17. The composition of claim 16, wherein the composition further comprises a vector comprising a polynucleotide encoding CAR binding the cell surface molecule of the WBC and a polynucleotide encoding IFNγ.

18. The composition of claim 17, wherein the composition further comprises a vector comprising a polynucleotide encoding CAR binding the cell surface molecule of the WBC and a polynucleotide encoding IL-6.

19. The composition of claim 18, wherein the composition further comprises a vector comprising a cytokine that is different from the cytokine of claim 17.

20. The composition of claim 19, wherein the cytokine is IL-12, IL-6, or IFNγ.

21. A method of preparing a mixed population of cells comprising contacting a population of cells with the composition of claim 8, thereby obtaining a mixed population of cells.

22. The method of claim 21, wherein the cells comprise NK cells and/or T cells.

23. The method of claim 21, wherein the cells comprise NK cells or T cells.
